SEQUENCE LISTING (30 sequences)

SEQ ID NO: 1 - protein, 11 aa, synthetic construct
KASQDVSIAVA

SEQ ID NO: 2 - protein, 7 aa, synthetic construct
SASYRYT

SEQ ID NO: 3 - protein, 9 aa, synthetic construct
QQHYITPLT

SEQ ID NO: 4 - protein, 5 aa, synthetic construct
NYGMN

SEQ ID NO: 5 - protein, 17 aa, synthetic construct
WINTYTGEPTYTDDFKG

SEQ ID NO: 6 - protein, 12 aa, synthetic construct
GGFGSSYWYFDV

SEQ ID NO: 7 - protein, 11 aa, synthetic construct
RASQGISGWLA

SEQ ID NO: 8 - protein, 7 aa, synthetic construct
AASTLQS

SEQ ID NO: 9 - protein, 9 aa, synthetic construct
QQANSFPPT

SEQ ID NO: 10 - protein, 5 aa, synthetic construct
SYNMN

SEQ ID NO: 11 - protein, 17 aa, synthetic construct
YISSSSSTIYYADSVKG

SEQ ID NO: 12 - protein, 8 aa, synthetic construct
AYYYGMDV

SEQ ID NO: 13 - protein, 214 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 14 - protein, 451 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 214 aa, synthetic construct
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 447 aa, synthetic construct
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 121 aa, synthetic construct
QVQLQQSGSELKKPGASVKVSCKASGYTFTNYGMNWVKQAPGQGLKWMGWINTYTGEPTYTDDFKGRFAFSLDTSVSTAYLQISSLKADDTAVYFCARGGFGSSYWYFDVWGQGSLVTVSS

SEQ ID NO: 18 - protein, 108 aa, synthetic construct
DIQLTQSPSSLSASVGDRVSITCKASQDVSIAVAWYQQKPGKAPKLLIYSASYRYTGVPDRFSGSGSGTDFTLTISSLQPEDFAVYYCQQHYITPLTFGAGTKVEIKR

SEQ ID NO: 19 - protein, 117 aa, synthetic construct
EVQLVESGGGLVQPGGSLRLSCAASGFTFSSYNMNWVRQAPGKGLEWVSYISSSSSTIYYADSVKGRFTISRDNAKNSLSLQMNSLRDEDTAVYYCARAYYYGMDVWGQGTTVTVSS

SEQ ID NO: 20 - protein, 107 aa, synthetic construct
DIQMTQSPSSVSASVGDRVTITCRASQGISGWLAWYQQKPGKAPKFLIYAASTLQSGVPSRFSGSGSGTDFTLTISSLQPEDFATYYCQQANSFPPTFGGGTKVEIK

SEQ ID NO: 21 - protein, 15 aa, synthetic construct
RASKGVSTSGYSYLH

SEQ ID NO: 22 - protein, 7 aa, synthetic construct
LASYLES

SEQ ID NO: 23 - protein, 9 aa, synthetic construct
QHSRDLPLT

SEQ ID NO: 24 - protein, 111 aa, synthetic construct [sequence omitted]

SEQ ID NO: 25 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 26 - protein, 5 aa, synthetic construct
NYYMY

SEQ ID NO: 27 - protein, 17 aa, synthetic construct
GINPSNGGTNFNEKFKN

SEQ ID NO: 28 - protein, 11 aa, synthetic construct
RDYRFDMGFDY

SEQ ID NO: 29 - protein, 120 aa, synthetic construct [sequence omitted]

SEQ ID NO: 30 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

(19) United States
(12) Patent Application Publication
(10) Pub. No.: US 2025/0135019 A1
(43) Pub. Date: May 1, 2025

(54) COMBINATION THERAPIES FOR TREATING CANCERS

(71) Applicant: Gilead Sciences, Inc., Foster City, CA (US)

(72) Inventors: Bradley A. McGregor, Boston, MA (US); Mitchell R. Sierecki, Cold Spring Harbor, NY (US); Guru P. Sonpavde, Boston, MA (US)

(21) Appl. No.: 18/913,762

(22) Filed: Oct. 11, 2024

Related U.S. Application Data
(60) Provisional application No. 63/651,111, filed on May 23, 2024.
(60) Provisional application No. 63/590,119, filed on Oct. 13, 2023.
(60) Provisional application No. 63/691,516, filed on Sep. 6, 2024.

(51) Int. Cl. A61K 47/68; A61K 9/00; A61P 35/04; C07K 16/28; C07K 16/30

(52) U.S. Cl. CPC A61K 47/68037; A61K 9/0019; A61K 47/68031; A61K 47/6849; A61K 47/6851; A61P 35/04; C07K 16/2803; C07K 16/2818; C07K 16/2827; C07K 16/30

(57) ABSTRACT

This disclosure provides methods for treating cancer in a patient, the methods including administering a combination therapy comprising dual antibody drug conjugates. In some embodiments the combination therapy comprises (i) a therapeutically effective amount of a Trop-2 antibody-drug conjugate (Trop-2 ADC), (ii) a therapeutically effective amount of a Nectin-4 antibody-drug conjugate (Nectin-4 ADC), and, optionally, (iii) a therapeutically effective amount of an anti-PD(L)-1 antibody. In some embodiments, the cancer is urothelial carcinoma.

### CROSS-REFERENCE TO RELATED APPLICATIONS

This application claims priority to U.S. Provisional Application No. 63/590,119, filed on Oct. 13, 2023, U.S. Provisional Application No. 63/651,111, filed on May 23, 2024, and U.S. Provisional Application No. 63/691,516, filed on Sep. 6, 2024, each of which is incorporated herein by reference in its entirety for all purposes.

### FIELD

The present disclosure relates to methods of treating cancer using combination treatments comprising two or more antibody-drug-conjugates (ADCs).

### SEQUENCE LISTING

The instant application contains a Sequence Listing which has been submitted electronically in .XML file format and is hereby incorporated by reference in its entirety. Said .XML copy, created on Sep. 24, 2024, is named 1521-US-NP_SL.xml and is 29,361 bytes in size.

### BACKGROUND

ADCs have emerged as a promising class of cancer therapeutics that can potentially be useful to treat a broad range of specific cancer types. See, e.g., Tsuchikama et al., Nat Rev Clin Oncol. 2024; 21(3): 203-223. ADCs are generally comprised of a monoclonal antibody that is connected to a potent cytotoxic payload through a chemical linker. By combining the target specificity and long circulation half-life of an antibody with the high cytotoxic potency of antitumor agents that are too toxic for monotherapy use ADCs can have enhanced antitumor efficacy, leading to improved clinical benefit and quality of life outcomes compared to conventional chemotherapies. To date, 11 ADCs have obtained regulatory approval in the U.S. for over 20 specific cancer indications, and over 100 ADCs are being studied in various clinical trials. Clinical stage ADCs are structurally highly diverse, targeting a variety of tumor-associated antigens, delivering payloads of different therapeutic modalities, and featuring a range of linker chemistries.

Urothelial carcinoma begins in the urothelial cells. The most common location for urothelial carcinoma is in bladder. Bladder cancer is estimated to kill nearly 200,000 patients globally on an annual basis.

Sacituzumab govitecan (“SG”) is an antibody-drug-conjugate (“ADC”) comprised of a (a) a humanized monoclonal antibody (“hRS7”) that binds to the cell-surface receptor, Trop 2, (b) a payload (“SN-38”) that is a topoisomerase I inhibitor, and (c) a linker (“CL2A”), that couples the antibody to the payload. SG is marketed under the name TRODELVY. The U.S. FDA has granted accelerated approval of SG as a monotherapy for use in patients with locally advanced or metastatic urothelial carcinoma who have previously received a platinum-containing chemotherapy and a PD(L)-1 inhibitor. A recently completed confirmatory Phase 3 study TROPiCS-04 in locally advanced or metatstatic urothelial carcinoma did not meet the primary endopoint of overall survival (OS) in the intention to treat population. A numerical improvement in OS favoring TRODELVY was observed, and trends in improvement for select pre-specified subgroups and secondary endpoints of progression-free survival (PFS) and overall response rate (ORR) were also shown.

Enfortumab vedotin (“EV”) is an ADC directed against nectin-4 with the microtubulin inhibitor monomethyl auristatin E (MMAE) payload. EV is marketed under the name PADCEV. The U.S. FDA has granted accelerated approval of EV as a monotherapy for use in patients with locally advanced or metastatic urothelial carcinoma who have previously received a platinum-containing chemotherapy and a PD(L)-1 inhibitor. Recently, the U.S. FDA has also granted accelerated approval to EV with pembrolizumab (brand name KEYTRUDA) for patients with locally advanced or metastatic urothelial carcinoma who are ineligible for cisplatin-containing chemotherapy.

Despite major progress in ADC mediated cancer therapies and continuing ADC development, the clinical potential of these drugs especially in patients with treatment-refractory cancers is often limited by factors such as tumor heterogeneity and the resulting emergence of ADC-resistant clones. Tolerability is also an important parameter for the successful development of an ADC. In fact, many ADCs have been withdrawn either from clinical studies or from the market ater initial approval owing to unacceptable toxicities or an overly narrow therapeutic window. For at least these reasons there remains a strong medical need for novel therapeutic approaches and improved ADC-based cancer treatment regimens.

### SUMMARY

One aspect of the present disclosure provides a method of treating of treating relapsed or refractory locally advanced or metastatic urothelial carcinoma in a human subject, including administering to the subject a combination therapy which includes a therapeutically effective amount of a Trop-2 antibody-drug conjugate (Trop-2 ADC); and a therapeutically effective amount of a Nectin-4 antibody-drug conjugate (Nectin-4 ADC).

Another aspect of the present disclosure provides a safe method of treating relapsed or refractory locally advanced or metastatic urothelial carcinoma in a human subject, comprising administering to the subject a combination therapy which includes a therapeutically effective amount of a Trop-2 antibody-drug conjugate (Trop-2 ADC); and a therapeutically effective amount of a Nectin-4 antibody-drug conjugate (Nectin-4 ADC).

Another aspect of the present disclosure provides a tolerable method of treating relapsed or refractory locally advanced or metastatic urothelial carcinoma in a human subject, comprising administering to the subject a combination therapy which includes a therapeutically effective amount of a Trop-2 antibody-drug conjugate (Trop-2 ADC); and a therapeutically effective amount of a Nectin-4 antibody-drug conjugate (Nectin-4 ADC).

Another aspect of the present disclosure provides a method of improving clinical efficacy compared to using a Trop-2 ADC or a Nectin-4 ADC alone treating relapsed or refractory locally advanced or metastatic urothelial carcinoma in a human subject, including administering to the subject a combination therapy which includes a therapeutically effective amount of a Trop-2 antibody-drug conjugate (Trop-2 ADC); and a therapeutically effective amount of a Nectin-4 antibody-drug conjugate (Nectin-4 ADC).

Another aspect of the present disclosure provides a method of without additional safety signals compared to using a Trop-2 ADC or a Nectin-4 ADC alone treating relapsed or refractory locally advanced or metastatic urothelial carcinoma in a human subject, including administering to the subject a combination therapy which includes a therapeutically effective amount of a Trop-2 antibody-drug conjugate (Trop-2 ADC); and a therapeutically effective amount of a Nectin-4 antibody-drug conjugate (Nectin-4 ADC).

Another aspect of the present disclosure provides a method of improving clinical efficacy compared to either SG or EV alone in a population of human subjects having locally advanced or metastatic UC that has relapsed after, or is refractory to, platinum-based chemotherapy and/or immunotherapy, comprising administering to the subject a combination therapy which comprises SG and EV.

Another aspect of the present disclosure provides a method of achieving a complete response in a human subject having locally advanced or metastatic urothelial carcinoma (“la/mUC”) for at least approximately 6 months, including a combination therapy including SG and EV.

In certain embodiments, the partial response is about 40% or greater reduction in the size of one or more measurable tumors from baseline to approximately 13 weeks or more from baseline.

In another aspect, provided herein is a method of treating locally advanced or metastatic urothelial carcinoma in a human subject, comprising administering to the subject a combination therapy which comprises: (i) a therapeutically effective amount of a Trop-2 antibody-drug conjugate (Trop-2 ADC) and (ii) a therapeutically effective amount of a Nectin-4 antibody-drug conjugate (Nectin-4 ADC).

In another aspect, provided herein is a Trop-2 ADC for use in a method of treating urothelial carcinoma in a human subject, said method comprising administering to the subject: (i) the Trop-2 ADC and (ii) a Nectin-4 ADC.

In another aspect, provided herein is a Nectin-4 ADC for use in a method of treating urothelial carcinoma in a human subject, said method comprising administering to the subject: (i) a Trop-2 ADC and (ii) the Nectin-4 ADC.

In another aspect, provided herein is a Trop-2 ADC and a Nectin-4 ADC for therapeutic use.

In another aspect, provided herein is a Trop-2 ADC and a Nectin-4 ADC for use in a method of treating urothelial carcinoma in a human subject.

In another aspect, provided herein is an anti-PD-1 antibody or anti-PD-L1 antibody for use in a method of treating urothelial carcinoma in a human subject, said method comprising administering to the subject: (i) a Trop-2 ADC; (ii) a Nectin-4 ADC, and (iii) the anti-PD-1 antibody or anti-PD-L1 antibody.

In another aspect, provided herein is a method of treating relapsed or refractory locally advanced or metastatic urothelial carcinoma in a human subject, comprising administering to the subject a combination therapy which comprises: (i) a therapeutically effective amount of a Trop-2 ADC and (ii) a therapeutically effective amount of a Nectin-4 ADC; wherein the combination therapy does not comprise an anti-PD1 antibody or an anti-PD-L1 antibody, and wherein the subject has received one or more prior therapies for locally advanced or metastatic urothelial carcinoma.

In another aspect, provided herein is a method of treating locally advanced or metastatic urothelial carcinoma in a human subject, comprising administering to the subject a combination therapy which comprises: (i) a therapeutically effective amount of a Trop-2 ADC; (ii) a therapeutically effective amount of a Nectin-4 ADC, and (iii) a therapeutically effective amount of an anti-PD1 antibody or an anti-PD-L1 antibody; wherein the subject has not received a prior therapy for locally advanced or metastatic urothelial carcinoma.

In another aspect, provided herein is a method of safely treating locally advanced or metastatic urothelial carcinoma in a human subject, comprising administering to the subject a combination therapy which comprises: (i) a therapeutically effective amount of a Trop-2 ADC, and (ii) a therapeutically effective amount of a Nectin-4 ADC.

In another aspect, provided herein is a method of treating locally advanced or metastatic urothelial carcinoma that is well tolerated in a human subject, comprising administering to the subject a combination therapy which comprises: (i) a therapeutically effective amount of a Trop-2 ADC and (ii) a therapeutically effective amount of a Nectin-4 ADC.

In another aspect, provided herein is a method of improving clinical efficacy of a reference Trop-2 ADC monotherapy or a reference Nectin-4 ADC monotherapy for treating locally advanced or metastatic urothelial carcinoma in a human subject, comprising administering to the subject a combination therapy which comprises: (i) a therapeutically effective amount of a Trop-2 ADC and (ii) a therapeutically effective amount of a Nectin-4 ADC.

In another aspect, provided herein is a method of improving clinical efficacy of a reference combination therapy comprising a reference Nectin-4 ADC and a reference anti-PD1 antibody for treating locally advanced or metastatic urothelial carcinoma in a human subject, comprising administering to the subject a combination therapy which comprises: (i) a therapeutically effective amount of a Trop-2 ADC and (ii) a therapeutically effective amount of a Nectin-4 ADC.

In another aspect, provided herein is a method of treating locally advanced or metastatic urothelial carcinoma in a human subject using a combination therapy without raising an additional safety signal compared to a reference Trop-2 ADC monotherapy or a reference Nectin-4 ADC monotherapy, comprising administering to the subject the combination therapy which comprises: (i) a therapeutically effective amount of a Trop-2 ADC and (ii) a therapeutically effective amount of a Nectin-4 ADC.

In another aspect, provided herein is a method of treating locally advanced or metastatic urothelial carcinoma in a human subject using a combination therapy without raising an additional safety signal compared to a reference combination therapy comprising a reference Nectin-4 ADC and a reference anti-PD-1 antibody, comprising administering to the subject the combination therapy, which comprises: (i) a therapeutically effective amount of a Trop-2 ADC; (ii) a therapeutically effective amount of a Nectin-4 ADC and (iii) a therapeutically effective amount of an anti-PD1 antibody or an anti-PD-L1 antibody.

In some embodiments, the reference Trop-2 ADC monotherapy comprises administering sacituzumab govitecan (SG). In some embodiments, the reference Trop-2 ADC monotherapy comprises administering SG at a dose of 10 mg/kg as an intravenous infusion on day 1 and day 8 of a 21-day treatment cycle.

In some embodiments, the reference Nectin-4 ADC or the reference Nectin-4 ADC monotherapy comprises enfortumab vedotin (EV). In some embodiments, the reference Nectin-4 ADC monotherapy comprises administering EV at a dose of 1.25 mg/kg as an intravenous infusion on day 1, day 8, and day 15 of a 28-day treatment cycle.

In some embodiments, the reference anti-PD-1 antibody is pembrolizumab. In some embodiments, the reference anti-PD-1 antibody comprising administering pembrolizumab at a dose of 200 mg as an intravenous infusion on day 1 of a 21-day treatment cycle.

In some embodiments, in the reference combination therapy the reference Nectin-4 ADC is reference EV; and reference EV is administered at a dose of 1.25 mg/kg as an intravenous infusion on day 1 and day 8 of one or more 21-day treatment cycles, and the reference anti-PD-1 antibody is reference pembrolizumab; and reference pembrolizumab is administered at a dose of 200 mg on day 1 of each of the one or more 21-day treatment cycles.

In some embodiments, the anti-Trop-2 antibody is sacituzumab or datopotamab. In some embodiments, the anti-Trop-2 antibody is sacituzumab. In some embodiments, the Trop-2 ADC comprises a topoisomerase I inhibitor. In some embodiments, the topoisomerase I inhibitor is SN38, exatecan, deruxtecan, or belotecan. In some embodiments, the topoisomerase I inhibitor is SN38. In some embodiments, the Trop-2 ADC is sacituzumab govitecan, sacituzumab tirumotecan (SKB264; sac-TMT), datopotamab deruxtecan (Dato-dxd), SHR-A1921, or ESG-401. In some embodiments, the Trop-2 ADC is sac-TMT. In some embodiments, the Trop-2 ADC is Dato-dxd. In some embodiments, the Trop-2 ADC is SG.

In some embodiments, the Nectin-4 ADC comprises enfortumab. In some embodiments, the Nectin-4 ADC comprises a microtubule inhibitor. In some embodiments, the microtubule inhibitor is a maytansinoid or auristatin. In some embodiments, the maytansinoid is maytansine, mertansine/emtansine (DM1) or raytansine/soravtansine (DM4). In some embodiments, the auristatin is monomethyl auristatin E (MMAE) or monomethyl auristatin F (MMAF). In some embodiments, the microtubule inhibitor is monomethyl auristatin E (MMAE). In some embodiments, the Nectin-4 ADC is enfortumab vedotin (EV), CRB-701, 9MW2821, ADRX-0706, BT-8009 (BicycleTX), SYS-6002 (CSPC Megalith Biopharmaceutical), or ARC-101 (ARS Pharmaceuticals). In some embodiments, the Nectin-4 ADC is enfortumab vedotin (EV).

In some embodiments, the Trop-2 ADC is administered as an intravenous infusion on day 1 and day 8 of a 21-day treatment cycle. In some embodiments, the Trop-2 ADC is administered at a dose of from 5 mg/kg to 10 mg/kg as an intravenous infusion on day 1 and day 8 of a 21-day treatment cycle. In some embodiments, the Trop-2 ADC is administered at a dose of 5 mg/kg as an intravenous infusion on day 1 and day 8 of a 21-day treatment cycle. In some embodiments, the Trop-2 ADC is administered at a dose of 7.5 mg/kg as an intravenous infusion on day 1 and day 8 of a 21-day treatment cycle. In some embodiments, the Trop-2 ADC is administered at a dose of 8 mg/kg as an intravenous infusion on day 1 and day 8 of a 21-day treatment cycle. In some embodiments, the Trop-2 ADC is administered at a dose of 10 mg/kg as an intravenous infusion on day 1 and day 8 of a 21-day treatment cycle.

In some embodiments, the Nectin-4 ADC is administered as an intravenous infusion on day 1 and day 8 of a 21-day treatment cycle. In some embodiments, the Nectin-4 ADC is administered at a dose of from 0.5 mg/kg to 1.25 mg/kg as an intravenous infusion on day 1 and day 8 a 21-day treatment cycle. In some embodiments, the Nectin-4 ADC is administered at a dose of 1 mg/kg as an intravenous infusion on day 1 and day 8 of a 21-day treatment cycle. In some embodiments, the Nectin-4 ADC is administered at a dose of 1.25 mg/kg as an intravenous infusion on day 1 and day 8 of a 21-day treatment cycle.

In some embodiments, the Trop-2 ADC is SG; and SG is administered at a dose of 5 mg/kg as an intravenous infusion on day 1 and day 8 of a 21-day treatment cycle; and the Nectin-4 ADC is EV; and EV is administered as an intravenous infusion at a dose of 1.25 mg/kg as an intravenous infusion on day 1 and day 8 of the 21-day treatment cycle.

In some embodiments, the Trop-2 ADC is SG; and SG is administered at a dose of 5 mg/kg as an intravenous infusion on day 1 and day 8 of a 21-day treatment cycle; and the Nectin-4 ADC is EV; and EV is administered as an intravenous infusion at a dose of 1 mg/kg as an intravenous infusion on day 1 and day 8 of the 21-day treatment cycle.

In some embodiments, the Trop-2 ADC is SG; and SG is administered at a dose of 5 mg/kg as an intravenous infusion on day 1 and day 8 of a 21-day treatment cycle; and the Nectin-4 ADC is EV; and EV is administered as an intravenous infusion at a dose of 0.75 mg/kg as an intravenous infusion on day 1 and day 8 of the 21-day treatment cycle.

In some embodiments, the Trop-2 ADC is SG; and SG is administered at a dose of 5 mg/kg as an intravenous infusion on day 1 and day 8 of a 21-day treatment cycles; and the Nectin-4 ADC is EV; and EV is administered as an intravenous infusion at a dose of 0.5 mg/kg as an intravenous infusion on day 1 and day 8 of the 21-day treatment cycle.

In some embodiments, the Trop-2 ADC is SG; and SG is administered at a dose of 7.5 mg/kg as an intravenous infusion on day 1 and day 8 of a 21-day treatment cycle; and the Nectin-4 ADC is EV; and EV is administered as an intravenous infusion at a dose of 1.25 mg/kg as an intravenous infusion on day 1 and day 8 of the 21-day treatment cycle.

In some embodiments, the Trop-2 ADC is SG; and SG is administered at a dose of 7.5 mg/kg as an intravenous infusion on day 1 and day 8 of a 21-day treatment cycle; and the Nectin-4 ADC is EV; and EV is administered as an intravenous infusion at a dose of 1 mg/kg as an intravenous infusion on day 1 and day 8 of the 21-day treatment cycle.

In some embodiments, the Trop-2 ADC is SG; and SG is administered at a dose of 7.5 mg/kg as an intravenous infusion on day 1 and day 8 of a 21-day treatment cycle; and the Nectin-4 ADC is EV; and EV is administered as an intravenous infusion at a dose of 0.75 mg/kg as an intravenous infusion on day 1 and day 8 of the 21-day treatment cycle.

In some embodiments, the Trop-2 ADC is SG; and SG is administered at a dose of 7.5 mg/kg as an intravenous infusion on day 1 and day 8 of a 21-day treatment cycle; and the Nectin-4 ADC is EV; and EV is administered as an intravenous infusion at a dose of 0.5 mg/kg as an intravenous infusion on day 1 and day 8 of the 21-day treatment cycle.

In some embodiments, the Trop-2 ADC is SG; and SG is administered at a dose of 8 mg/kg as an intravenous infusion on day 1 and day 8 of a 21-day treatment cycle; and the Nectin-4 ADC is EV; and EV is administered as an intravenous infusion at a dose of 1.25 mg/kg on day 1 and day 8 of the 21-day treatment cycles.

In some embodiments, the Trop-2 ADC is SG; and SG is administered at a dose of 8 mg/kg as an intravenous infusion on day 1 and day 8 of a 21-day treatment cycle; and the Nectin-4 ADC is EV; and EV is administered as an intravenous infusion at a dose of 1 mg/kg as an intravenous infusion on day 1 and day 8 of the 21-day treatment cycle.

In some embodiments, the Trop-2 ADC is SG; and SG is administered at a dose of 8 mg/kg as an intravenous infusion on day 1 and day 8 of a 21-day treatment cycles; and the Nectin-4 ADC is EV; and EV is administered as an intravenous infusion at a dose of 0.75 mg/kg as an intravenous infusion on day 1 and day 8 of the 21-day treatment cycle.

In some embodiments, the Trop-2 ADC is SG; and SG is administered at a dose of 8 mg/kg as an intravenous infusion on day 1 and day 8 of a 21-day treatment cycles; and the Nectin-4 ADC is EV; and EV is administered as an intravenous infusion at a dose of 0.5 mg/kg as an intravenous infusion on day 1 and day 8 of the 21-day treatment cycle.

In some embodiments, the Trop-2 ADC is SG; and SG is administered at a dose of 10 mg/kg as an intravenous infusion on day 1 and day 8 of a 21-day treatment cycle; and the Nectin-4 ADC is EV; and EV is administered as an intravenous infusion at a dose of 1.25 mg/kg as an intravenous infusion on day 1 and day 8 of the 21-day treatment cycle.

In some embodiments, the Trop-2 ADC is SG; and SG is administered at a dose of 10 mg/kg as an intravenous infusion on day 1 and day 8 of a 21-day treatment cycle; and the Nectin-4 ADC is EV; and EV is administered as an intravenous infusion at a dose of 1 mg/kg as an intravenous infusion on day 1 and day 8 of the 21-day treatment cycle.

In some embodiments, the Trop-2 ADC is SG; and SG is administered at a dose of 10 mg/kg as an intravenous infusion on day 1 and day 8 of a 21-day treatment cycle; and the Nectin-4 ADC is EV; and EV is administered as an intravenous infusion at a dose of 0.75 mg/kg as an intravenous infusion on day 1 and day 8 of the 21-day treatment cycle.

In some embodiments, the Trop-2 ADC is SG; and SG is administered at a dose of 10 mg/kg as an intravenous infusion on day 1 and day 8 of a 21-day treatment cycle; and the Nectin-4 ADC is EV; and EV is administered as an intravenous infusion at a dose of 0.5 mg/kg as an intravenous infusion on day 1 and day 8 of the 21-day treatment cycle.

In some embodiments, the subject has metastatic urothelial carcinoma. In some embodiments, the urothelial carcinoma is relapsed or refractory locally advanced or metastatic urothelial carcinoma.

In some embodiments, the subject is treatment naïve. In some embodiments, the subject has not received a prior systemic therapy for metastatic or locally advanced urothelial carcinoma. In some embodiments, the subject is ineligible for or refusing cisplatin based therapy. In some embodiments, the subject has received one or more prior therapies for metastatic or locally advanced urothelial carcinoma. In some embodiments, the metastatic or locally advanced urothelial carcinoma has progressed during or after each of the one or more prior therapies. In some embodiments, the subject has received prior platinum-based therapy for metastatic urothelial carcinoma, and the metastatic urothelial carcinoma has progressed during or after the platinum-based therapy. In some embodiments, the subject has received prior immunotherapy for metastatic urothelial carcinoma, and the metastatic urothelial carcinoma has progressed during or after the immunotherapy. In some embodiments, the locally advanced or metastatic urothelial carcinoma has relapsed after, or is refractory to, platinum-based chemotherapy and/or an anti-PD-1 antibody or anti-PD-L1 antibody.

In some embodiments, the combination therapy does not comprise an additional systemic therapy for locally advanced or metastatic urothelial carcinoma. In some embodiments, the combination therapy does not comprise an anti-PD-1 antibody or anti-PD-L1 antibody. In some embodiments, the combination therapy further comprises an anti-PD-1 antibody or anti-PD-L1 antibody. In some embodiments, the anti-PD-L1 antibody is selected from atezolizumab, avelumab, durvalumab, cosibelimab, lodapolimab, budigalimab, avelumab, and envafolimab.

In some embodiments, the combination therapy further comprises an anti-PD-1 antibody. In some embodiments, the anti-PD-1 antibody is selected from pembrolizumab, nivolumab, cemiplimab, pidilizumab, spartalizumab, sasanlimab, tislelizumab, retifanlimab, balstilimab, toripalimab, cetrelimab, genolimzumab, prolgolimab, camrelizumab, dostarlimab, sintilimab, and zimberelimab. In some embodiments, the anti-PD-1 antibody is pembrolizumab.

In some embodiments, pembrolizumab is administered to the subject at a dose level of about 200 mg per dose. In some embodiments, pembrolizumab is administered once every 21-day treatment cycle. In some embodiments, pembrolizumab is administered on day 1 of the one or more 21-day treatment cycles. In some embodiments, pembrolizumab is administered to the subject at a dose level of about 400 mg per dose. In some embodiments, pembrolizumab is administered on day 1 of every other 21-day treatment cycle. In some embodiments, the anti-PD-1 antibody is zimberelimab. In some embodiments, zimberelimab is administered at a dose level in the range of 300 mg to 400 mg. In some embodiments, zimberelimab is administered at a dose level of about 360 mg.

In some embodiments, zimberelimab is administered once every 21-day treatment cycle. In some embodiments, zimberelimab is administered on day 1 of the one or more 21-day treatment cycles. In some embodiments, the combination therapy is administered until disease progression or unacceptable toxicity. In some embodiments, the combination therapy is administered for 10 or more, 11 or more, 12 or more, 13 or more, 14 or more, 15 or more, 16 or more, 17 or more, 18 or more, 19 or more, or 20 or more treatment cycles. In some embodiments, the combination therapy is administered for 14 or more treatment cycles.

In some embodiments, when the method is used to treat a population of subjects having locally advanced or metastatic UC, the method achieves an overall response rate (ORR) of at least 60%, at least 65%, or at least 70%. In some embodiments, when the method is used to treat a population of subjects having locally advanced or metastatic UC, the method achieves an overall response rate (ORR) of 60-85%, 65-85%, 70-85%, 70-80%, or 70-75%. In some embodiments, the Trop-2 ADC is SG and the Nectin-4 ADC is EV, wherein when the method is used to treat a population of subjects, the method achieves an overall response rate (ORR) of at least 60% at a median follow-up of 14 months or more. In some embodiments, when the method is used to treat a population of subjects, the method achieves a higher ORR compared to administering the Trop-2 ADC or the Nectin-4 ADC as a reference monotherapy to a population of reference subjects. In some embodiments, when the method is used to treat a population of subjects, the method achieves a higher ORR compared to administering a reference combination therapy comprising EV and pembrolizumab to a population of reference subjects.

In some embodiments, when the method is used to treat a population of subjects, the method achieves a median duration of response (DOR) of greater than 12 months. In some embodiments, the method achieves a median DOR greater than 14 months. In some embodiments, when the method is used to treat a population of subjects, the 12-month progression free survival rate of the population is at least 40%. In some embodiments, the 12-month progression free survival rate of the population is 40%-80%. In some embodiments, the 12-month progression free survival rate of the population is 40%-60%. In some embodiments, the 12-month progression free survival rate of the population is 40%-50%. In some embodiments, the 12-month progression free survival rate of the population is 40%-45%.

In another aspect, provided herein is a method of improving clinical efficacy of either a reference SG monotherapy or a reference EV monotherapy in a population of human subjects having locally advanced or metastatic UC that has relapsed after, or is refractory to, platinum-based chemotherapy and/or immunotherapy, comprising administering to each subject in the population a combination therapy which comprises a therapeutically effective amount of SG and a therapeutically effective amount of EV. In some embodiments, the method achieves an ORR of at least 70%. In some embodiments, the method achieves an ORR of 70%-80%. In some embodiments, the method achieves a median duration of response (DOR) of greater than 12 months. In some embodiments, the method achieves a median DOR greater than 14 months.

In another aspect, provided herein is a method of achieving an improved durability of clinical response in a population of human subjects having locally advanced or metastatic UC that has relapsed after, or is refractory to, platinum-based chemotherapy and/or immunotherapy, compared to either a reference SG monotherapy or a reference EV monotherapy, comprising administering to each subject of the population of human subjects a combination therapy which comprises a therapeutically effective amount of SG and a therapeutically effective amount of EV.

In some embodiments of the methods provided herein, a 12-month progression free survival rate is achieved of at least 40%. In some embodiments, a 12-month progression free survival rate is a 40%-80%. In some embodiments, a 12-month progression free survival rate is 40%-60%. In some embodiments, a 12-month progression free survival rate is a 40%-50%. In some embodiments, a 12-month progression free survival rate is a 40%-45%.

In another aspect, provided herein is a method of achieving a complete response in a human subject having mUC for at least approximately 6 months, comprising administering to the subject a combination therapy comprising a therapeutically effective amount of SG and a therapeutically effective amount of EV.

In some embodiments of the methods provided herein, when the method is used to treat a population of subjects, the method achieves less than about 20% serious treatment related adverse events (TRAEs). In some embodiments, when the method is used to treat a population of subjects, the method achieves less than about 60% TRAEs of grade ≥3. In some embodiments, when the method is used to treat a population of subjects, the method achieves less than about 70% TRAEs of grade ≥3. In some embodiments, when the method is used to treat a population of subjects, the method achieves less than about 80% TRAEs of grade ≥3. In some embodiments, when the method is used to treat a population of subjects, less than 20% subjects discontinue the therapy due to TRAE. In some embodiments, when the method is used to treat a population of subjects, less than 30% subjects discontinue the therapy due to TRAE. In some embodiments, when the method is used to treat a population of subjects, less than 40% subjects discontinue the therapy due to TRAE. In some embodiments, when the method is used to treat a population of subjects, less than 50% subjects discontinue the therapy due to TRAE. In some embodiments, the safety or tolerability of the method is indicated by subjects' rates of discontinuation of treatment due to TRAE, and wherein subjects' rate of discontinuation is less than 20%, 30%, 40%, or 50%. In some embodiments, the TRAE is selected from anemia, febrile neutropenia, chest pain—cardiac, supraventricular tachycardia, abdominal pain, diarrhea, enterocolitis, ileal obstruction, mucositis oral, nausea, small intestinal obstruction, fatigue, pain, abdominal infection, enterocolitis infectious, infections and infestations, sepsis, skin infection, upper respiratory infection, urinary tract infection, wound infection, lipase increased, lymphocyte count decreased, neutrophil count decreased, platelet count decreased, white blood cell decreased, anorexia, dehydration, hyperglycemia, hypoalbuminemia, hypokalemia, acute kidney injury, urinary tract obstruction, hiccups, pneumonitis, respiratory, thoracic and mediastinal disorders, hypertension, hypotension, and thromboembolic event.

In another aspect, provided herein is a method of treating relapsed or refractory locally advanced or metastatic urothelial carcinoma in a population of human subjects, comprising administering to each subject in the population a combination therapy which comprises: administering SG at a dose of 8 mg/kg as an intravenous infusion on day 1 and day 8 of one or more 21-day treatment cycles; and administering EV as an intravenous infusion at a dose 1.25 mg/kg on day 1 and day 8 of one or more 21-day treatment cycles; wherein the combination therapy achieves ORR of 65-75%.

In another aspect, provided herein is a method of treating relapsed or refractory locally advanced or metastatic urothelial carcinoma in a population of human subjects, comprising administering to each subject in the population a combination therapy which comprises: administering SG at a dose of 7.5 mg/kg as an intravenous infusion on day 1 and day 8 of one or more 21-day treatment cycles; and administering EV as an intravenous infusion at a dose 1.25 mg/kg on day 1 and day 8 of one or more 21-day treatment cycles; wherein the method achieves an ORR of 65-75%. In some embodiments, the combination therapy has a manageable safety profile. In some embodiments, subjects' rates of discontinuation of the combination therapy due to TRAE is less than 20%, 30%, 40%, or 50%. In some embodiments, when the method is used to treat a population of subjects, the method achieves less than about 80% TRAEs of grade ≥3. In some embodiments, the TRAE is selected from anemia, febrile neutropenia, chest pain—cardiac, supraventricular tachycardia, abdominal pain, diarrhea, enterocolitis, ileal obstruction, mucositis oral, nausea, small intestinal obstruction, fatigue, pain, abdominal infection, enterocolitis infectious, infections and infestations, sepsis, skin infection, upper respiratory infection, urinary tract infection, wound infection, lipase increased, lymphocyte count decreased, neutrophil count decreased, platelet count decreased, white blood cell decreased, anorexia, dehydration, hyperglycemia, hypoalbuminemia, hypokalemia, acute kidney injury, urinary tract obstruction, hiccups, pneumonitis, respiratory, thoracic and mediastinal disorders, hypertension, hypotension, and thromboembolic event. In some embodiments, the combination therapy achieves a median duration of response (DOR) of greater than 12 months. In some embodiments, the combination therapy achieves a 12-month progression free survival (PFS) rate of at least 40%. In some embodiments, the subject has received prophylactic granulocyte colony stimulating factor.

In some embodiments of the methods provided herein, the subject has received premedication comprising antipyretics, H1 blocker, and H2 blocker. In some embodiments, the antipyretics is acetaminophen; the H1 blocker is Benadryl or Loratidine; and the H2 blocker is Pepcid. In some embodiments, the subject has received acetaminophen 650 mg PO; Benadryl 25 mg PO/IV or Loratidine 10 mg PO; and H2 blocker Pepcid 20 mg PO/IV. In some embodiments, the subject has received antiemetic premedication comprising a 5-HT3 inhibitor, an NK1-receptor antagonist, and dexamethasone. In some embodiments, the 5-HT3 inhibitor is ondansetron or palonosetron; and the NK1-receptor antagonist is fosaprepitant or aprepitant. In some embodiments, the premedication is administered approximately 30 minutes before each SG administration.

In some embodiments of the methods provided herein, EV and SG are administered sequentially in the order of first EV, then SG. In some embodiments, EV, pembrolizumab, and SG are administered sequentially in the order of first EV, pembrolizumab second, then SG.

### BRIEF DESCRIPTION OF THE DRAWINGS

FIG. 1 shows a Flow Diagram for the phase I double antibody drug conjugate (DAD) trial combining SG+EV for mUC. Patients in the “physician's decision” category for treatment discontinuation had achieved a durable response but came off treatment due to cumulative toxicity concerns.

FIG. 2 shows swimmer's plot of 23 treated patients on the phase I DAD trial combining SG+EV. (DL=dose level; BOR=best overall response; PR=partial response; CR=complete response; SD=stable disease; PD=progressive disease; NE=not evaluated. * The treatment cycle was 3 weeks, and radiographic imaging was performed at baseline, every 6 weeks during the first 4 months, and then every 9 weeks thereafter.)

FIG. 3 shows degree of shrinkage in target lesions.

### DETAILED DESCRIPTION

Aspects of the present disclosure relate to methods for treating a cancer in a patient including administering a combination therapy comprising (i) a therapeutically effective amount of a Trop-2 antibody-drug conjugate (Trop-2 ADC) and (ii) a therapeutically effective amount of a Nectin-4 antibody-drug conjugate (Nectin-4 ADC). In some embodiments, the Trop-2 ADC comprises a topoisomerase I inhibitor payload. In some embodiments, the Trop-2 ADC is sacituzumab govitecan (SG). In some embodiments, the Nectin-4 ADC comprises a microtubule inhibitor payload. In some embodiments, the Nectin-4 ADC is enfortumab vedotin (EV). In some embodiments, the anti-PD-1 antibody is pembrolizumab. In some embodiments the anti-PD-1 antibody is zimberelimab. In some embodiments, the combination therapy does not comprise an additional anticancer agent. In some embodiments, the combination therapy further comprises (iii) a therapeutically effective amount of an anti-PD-1 antibody or of an anti-PD-L1 antibody (an anti-PD(L)-1 antibody). In some embodiments the cancer is locally advanced or metastatic urothelial carcinoma. In some embodiments the cancer is relapsed or refractory locally advanced or metatstatic urothelial carcinoma. In some embodiments, the patient has not received a prior treatment for locally advanced or metastatic urothelial carcinoma. In some embodiments, the patient has received one or more prior treatments for locally advanced or metastatic urothelial carcinoma. In some embodiments, the patient has received prior platinum-based therapy for metastatic urothelial carcinoma, and the metastatic urothelial carcinoma has progressed during or after the platinum-based therapy. In some embodiments, the patient has received prior immunotherapy for metastatic urothelial carcinoma, and the metastatic urothelial carcinoma has progressed during or after the immunotherapy. In some embodiments, the cancer has relapsed after, or is refractory to, platinum-based chemotherapy and/or an anti-PD-1 antibody or anti-PD-L1 antibody. In some embodiments, the efficacy of a combination therapy provided herein is improved relative to a reference combination therapy including the Trop-2 ADC or the Nectin-4 ADC and an anti-PD(L)1 antibody (e.g., an anti-PD-1 antibody or anti-PD-L1 antibody).

This disclosure is, at least in part, based on the recognition that a combination therapy comprising (i) a Trop-2 ADC including a topoisomerase inhibitor payload and (ii) a Nectin-4 ADC including a microtubule inhibitor payload, and optionally further comprising (iii) an anti-PD-1 antibody or anti-PD-L1 antibody (anti-PD(L)-1 antibody) can improve the efficacy of anticancer reference therapies, such as a Trop-2 ADC reference monotherapy, a Nectin-4 ADC reference monotherapy, or a Nectin-4 ADC plus anti-PD(L)-1 antibody reference combination therapy.

This disclosure is further, at least in part, based on the recognition that a combination therapy comprising (i) a Trop-2 ADC including a topoisomerase inhibitor payload and (ii) a Nectin-4 ADC including a microtubule inhibitor payload, and optionally further comprising (iii) an anti-PD-1 antibody or anti-PD-L1 antibody (anti-PD(L)-1 antibody) can be safely administered to cancer patients, and observed adverse events are manageable by skilled clinicians using standard of care risk mitigation methods. In some embodiments, the combination therapies disclosed herein do not produce adverse events other than those expected for reference therapies involving the Trop-2 ADC, Nectin-4 ADC, and, optionally, the anti-PD(L)1 antibody administered as part of the disclosed combination therapy. This disclosure is further, at least in part, based on the recognition that SG and EV specifically have non-overlapping major toxicities. While EV is primarily associated with neurotoxicity, rash, and hyperglycemia, the main toxicities of SG include myelosuppression and diarrhea.

### Definitions

Unless otherwise defined herein, scientific and technical terms used in connection with the present application shall have the meanings that are commonly understood by those of ordinary skill in the art. Further, unless otherwise required by context, singular terms shall include pluralities and plural terms shall include the singular.

The singular forms “a,” “an,” and “the” include the plural referents unless the context dictates otherwise.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, such as, for example, within 15%, 10%, or 5%.

“Actionable genomic alteration(s),” is known in the art, i.e., a gene is actionable if it has an established biologic role in cancer and there is a clinically available drug to which the gene confers sensitivity or resistance, where actionability can be applicable to all or select alteration or tumor types.

“Advanced disease” refers to stage III disease which has not spread or cancers that are unlikely to be cured or controlled long-term with treatment, as they have spread to distant locations.

“Anti-cancer agent” refers to an approved or investigational agent(s) for use, or that are being evaluated in, cancer. It can refer to systemic and targeted therapy.

“Metastatic cancer or disease” is a sub-category of advanced disease. It refers to Stage IV disease and/or cancers that are unlikely to be cured or controlled long-term with treatment, as they have spread to distant locations.

“Locally advanced disease,” as used herein, refers to a disease state in which cancer cells have begun to spread out from the initial site of origin but have not yet spread to other parts of the body. In certain embodiments, “locally advanced” refers to cancers that are unresectable.

“Antibody-drug conjugate” or “ADC” generally refers to a compound comprising an antibody targeting a tumor antigen and an anticancer agent drug or payload, optionally connected by a linker. In some embodiments, the ADC is a Trop-2 ADC. In some embodiments the ADC is a Nectin-4 ADC. In certain embodiments, the ADC is SG. In certain embodiments, the ADC is an ADC that is approved for metastatic urothelial carcinoma (mUC), or in the clinic being evaluated for mUC. In certain embodiments, the ADC comprises a Trop2-targeting antibody. In certain embodiments, “ADC” refers to the compound of Formula I, which has an anti-Trop-2 antibody (sacituzumab) linked to the drug or payload SN38. In certain embodiments, the ADC is EV.

An “antibody fragment” or “antigen binding fragment” refers to a molecule other than an intact antibody that comprises a portion of an intact antibody that binds the antigen to which the intact antibody binds. The antibody fragment retains the ability to bind specifically to the antigen bound by the full-length antibody, e.g., fragments that retain one or more CDR regions, e.g., all six CDRs. Examples of antibody fragments include but are not limited to Fv, Fab, Fab′, Fab′-SH, F(ab′)₂; diabodies; linear antibodies; single-chain antibody molecules (e.g., scFv); and multi-specific antibodies formed from antibody fragments.

An antibody that “specifically binds to” a specified target protein is an antibody that exhibits preferential binding to that target as compared to other proteins, but this specificity does not require absolute binding specificity. An antibody is considered “specific” for its intended target if its binding is determinative of the presence of the target protein in a sample, e.g., without producing undesired results such as false positives. Antibodies, or binding fragments thereof, useful in the present invention will bind to the target protein with an affinity that is at least two-fold greater, preferably at least ten times greater, more preferably at least 20-times greater, and most preferably at least 100-times greater than the affinity with non-target proteins. As used herein, an antibody is said to bind specifically to a polypeptide comprising a given amino acid sequence, e.g., the amino acid sequence of a mature human PD-1 or human PD-L1 molecule, if it binds to polypeptides comprising that sequence but does not bind to proteins lacking that sequence.

“Baseline” as used herein, is the time within approximately 28 days prior to initiation of therapy, i.e., within approximately 28 days prior to day 1 of cycle 1. In certain embodiments, baseline refers to approximately day −28 to day −1 prior to day 1 of cycle 1. In certain embodiments, the baseline is the time within approximately 28 days+/−7 days prior to the initiation of therapy. In certain embodiments, baseline is the time from one measurement to another, i.e., baseline can refer to an initial time point when a measurement, i.e., a measurement of a lesion, is compared between two time points. The initial time point, in this instance, does not necessarily correspond with the initiation of treatment.

“Chemotherapy-naïve,” as used herein, refers to not having received chemotherapy, i.e., not having received chemotherapy for locally advanced or metastatic urothelial carcinoma.

“Combination therapy” refers to the concurrent administration of two or more drugs or therapies to a patient. In some embodiments, “combination therapy” refers to administration of two or more drugs during the same time period. In certain embodiments, “combination therapy” refers to administration of two or more drugs during the same dosing cycle. For example, if the dosing cycle is 21-days, then “combination therapy” can refer to administration of drug 1 on day 1, and drug 2 on day 1 or any other day within the 21-day dosing cycle. In certain embodiments, “combination therapy” refers to two or more drugs that are administered together within at least one dosing cycle. The two or more drugs of the combination therapy can be administered at the same time (e.g., through intravenous infusion from the same IV bag) or successively, with or without an intervening time period between administrations. The combination therapy of the two therapies can also be via the same or different routes of administration. In some embodiments, the combination therapy is administered in a 21-day cycle with SG and EV given as an intravenous fusion (IV) on Days 1 and 8 of each 3-week cycle.

“Clinical efficacy” or “clinical activity” refers to clinical efficacy or activity in human patient(s). In certain embodiments, clinical activity or clinical efficacy refers to a partial or complete response. In certain embodiments, clinical efficacy or clinical activity, when used in the context of a population of patients, comprises ORR, DOR, and/or DCR. In certain embodiments, improvement of clinical efficacy or clinical activity, when used in the context of a comparison between two or more agents, can be used to describe a clinically meaningful benefit to the human patient of one agent compared to the other. As used herein “clinically meaningful benefit” (or grammatical variations thereof) refers to results/findings that improve medical care resulting in improvement in the human patient's physical function, his/her mental status, and/or ability to engage in social life. The term improvement of quality of life in medical care deals with both subjective and objective terms. Objective terms may be duration of remission of disease, etc. Subjective terms may be improvement in the quality of life.

“Complete Response” (“CR”) refers to disappearance of all target lesions. Any pathological lymph nodes (whether target or non-target) must have reduction in short axis to <10 mm.

“CDR” or “CDRs” as used herein means complementarity determining region(s) in an immunoglobulin variable region, defined using the Kabat numbering system, unless otherwise indicated.

“Concomitant administration” or “concurrent administration,” can be used interchangeably, and refer to administration of two or more drugs or therapies during the same dosing cycle. Concurrent administration of two or more drugs of a combination therapy disclosed herein can include the simultaneous administration of two or more drugs (e.g., through intravenous infusion from the same IV bag), or the independent administration of the two or more drugs, e.g., on the same day (e.g., successively with or without an intervening time period), or on different days of a dosing cycle. Each of the drugs of a combination therapy disclosed herein can be approved or investigational for the indication-of-interest.

“Duration of response” (“DOR”) refers to the time (e.g., months) from onset of response to progression or death. In some embodiments, the duration of response is measured from the time measurement criteria are met for CR or PR (whichever is first recorded) until the first date that recurrent or progressive disease is objectively documented (taking as reference for progressive disease the smallest measurements recorded since the treatment started, or death due to any cause. Participants without events reported are censored at the last disease evaluation).

“First line treatment” as is known in the art refers to the initial therapy for a particular disease.

“Immunotherapy” refers to treatment of disease (e.g., cancer) by modulating an immune response to a disease antigen. As used herein, immunotherapy refers to providing an anti-cancer immune response in a subject by administration of an agent that elicits an anti-tumor antigen immune response in the subject.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

“Monotherapy” as used herein refers to the use of a single method or a single therapeutic agent to treat a cancer. “SG monotherapy” as used herein refers to the use of SG as a single method or therapeutic agent to treat a cancer. In some embodiments, “SG monotherapy” refers to administering SG as an intravenous infusion 10 mg/kg once weekly on Days 1 and 8 of continuous 21-day treatment cycles until disease progression or unacceptable toxicity. “EV monotherapy” as used herein refers to the use of EV as a single method to treat a cancer. In some embodiments, “EV monotherapy” refers to administering EV 1.25 mg/kg (up to a maximum dose of 125 mg) as an intravenous infusion over 30 minutes on Days 1, 8 and 15 of a 28-day cycle until disease progression or unacceptable toxicity.

“Nectin-4 antibody drug conjugate” or “Nectin-4 ADC” refers to an anti-Nectin-4 antibody or antigen binding fragment thereof conjugated or recombinantly fused to a therapeutic moiety (or one or more therapeutic moieties), or an anti-Nectin-4 antibody or antigen binding fragment thereof conjugated or recombinantly fused to a therapeutic moiety or drug moiety (a “payload”) that modifies a given biological response, as described above. In some embodiments, the Nectin-4 ADC comprises a microtubule inhibitor payload. In some embodiments, the microtubule inhibitor is a maytansinoid or auristatin. In some embodiments the maytansinoid is maytansine, mertansine/emtansine (DM1) or raytansine/soravtansine (DM4). In some embodiments the auristatin is monomethyl auristatin E (MMAE) or monomethyl auristatin F (MMAF). In some embodiments, the microtubule inhibitor is monomethyl auristatin E (MMAE). In some embodiments, the Nectin-4 ADC is enfortumab vedotin (EV) (Seagen/Pfizer), CRB-701 (Corbus Pharmaceuticals), 9MW2821 (Mabwell), ADRX-0706 (Adcentrx), BT-8009 (BicycleTX), SYS-6002 (CSPC Megalith Biopharmaceutical), or ARC-101 (ARS Pharmaceuticals).

“Objective Responsive Rate” (“ORR”) refers to number of patients achieving objective response divided by number of patients who initiate treatment. Objective response is defined as best overall response of complete response (CR) or partial response (PR).

“Partial Response” (“PR”), refers to at least a 30% decrease in the sum of the diameters of target lesions, taking as reference the baseline sum diameters.

“Patient” or “subject,” as used interchangeably herein, refer to an animal, such as a mammal (including a human), that has been or will be the object of treatment, observation or experiment. In some embodiments, the subject (or the patient) is human, domestic animals (e.g., dogs and cats), farm animals (e.g., cattle, horses, sheep, goats, and pigs), and/or laboratory animals (e.g., mice, rats, hamsters, guinea pigs, pigs, rabbits, dogs, and monkeys). In some embodiments, the subject (or the patient) is a human. “Human subject,” as used herein, refers to a human having a particular disease. In certain embodiments, the patient is an adult human patient. In certain embodiments, the patient is a pediatric human patient.

“Pembrolizumab” (formerly known as MK-3475, SCH 900475, and lambrolizumab), alternatively referred to herein as “pembro,” is a humanized IgG4 mAb with the structure described in WHO Drug Information, Vol. 27, No. 2, pages 161-162 (2013) and which comprises the heavy and light chain amino acid sequences and CDRs described in Table 3. Pembrolizumab has been approved by the U.S. FDA as described in the Prescribing Information for KEYTRUDA™ (Merck & Co., Inc., Rahway, NJ, USA; initial U.S. approval 2014, updated March 2021).

As used herein, a “pembrolizumab variant” or “a variant thereof” pertaining to a pembrolizumab sequence means a monoclonal antibody that comprises heavy chain and light chain sequences that are substantially identical to those in pembrolizumab, except for having three, two or one conservative amino acid substitutions at positions that are located outside of the light chain CDRs and six, five, four, three, two or one conservative amino acid substitutions that are located outside of the heavy chain CDRs, e.g., the variant positions are located in the FR regions or the constant region, and optionally has a deletion of the C-terminal lysine residue of the heavy chain. In other words, pembrolizumab and a pembrolizumab variant comprise identical CDR sequences, but differ from each other due to having a conservative amino acid substitution at no more than three or six other positions in their full-length light and heavy chain sequences, respectively. A pembrolizumab variant is substantially the same as pembrolizumab with respect to the following properties: binding affinity to PD-1 and ability to block the binding of each of PD-L1 and PD-L2 to PD-1.

“Platinum-ineligible urothelial carcinoma” refers to any mUC patient meeting one the following 5 parameters: 1) ECOG PS≥3; 2) creatinine clearance (Cr Cl)<30 ml/min; 3) peripheral neuropathy ≥Grade 2; 4) NYHA Heart Failure Class >3; or 5) ECOG PS 2 AND Cr Cl 30 to 59 ml/min. In some embodiments, the platinum-ineligible patients are cisplatin-ineligible. In some embodiments, the platinum-ineligible patients are carboplatin-ineligible.

“Progression Free Survival” (“PFS”) refers to the time from registration to the earlier of progression per investigator assessment or death due to any cause. Participants alive without disease progression are censored at date of last disease evaluation.

“Reference therapy” refers to a therapy comprising the administration of a Trop-2 ADC, Nectin-4 ADC, or, optionally, an anti-PD(L)-1 antibody described herein to a human subject that is in accordance with a dose and schedule described on a drug label (e.g., FDA-approved Prescribing Information; United States Prescribing Information) for an indicated use. Alternatively, if a reference use is not indicated for a clinical setting on a drug label “reference therapy” refers to a therapy comprising the administration of a Trop-2 ADC, Nectin-4 ADC, or, optionally, an anti-PD(L)-1 antibody described herein to a human subject that mirrors a dose and schedule used in a clinical trial in a comparable clinical setting. In some embodiments, a Trop-2 ADC reference monotherapy comprises administering SG to a human patient with locally advanced or metastatic urothelial carcinoma at a dose of 10 mg/kg as an intravenous infusion on day 1 and day 8 of a 21-day treatment cycle. In some embodiments, a Nectin-4 ADC/anti-PD-1 antibody reference combination therapy comprises administering EV at a dose of 1.25 mg/kg as an intravenous infusion on day 1 and day 8 of a 21-day treatment cycle, and administering pembrolizumab at a dose of 200 mg on day 1 of the 21-day treatment cycle. In some embodiments, the reference Trop-2 ADC is SG. In some embodiments, the reference Nectin-4 ADC is EV and the reference anti-PD-1 antibody is pembrolizumab.

“Refractory” is used to describe when the disease does not respond to treatment or when the response to treatment does not last very long.

“Relapsed” refers to a disease that reappears or grows again after a period of remission.

A “Trop-2 antibody-drug conjugate (Trop-2 ADC)” refers to a compound comprising an antibody targeting tumor-associated calcium signal transducer 2 (Trop-2; NCBI Gene ID: 4070) and an anticancer agent payload, optionally connected by a linker. In some embodiments, the tumor antigen targeted antibody is an anti-Trop-2 antibody (e.g., sacituzumab or datopotamab). In some embodiments, the Trop-2 ADC comprises a topoisomerase I payload. In some embodiments the topoisomerase I payload is SN38, exatecan, deruxtecan, or belotecan. In certain embodiments, “ADC” refers to the compound of Formula I, which has an anti-Trop-2 antibody (sacituzumab) linked to the drug or payload SN38. In some embodiments, the Trop-2 ADC is sacituzumab govitecan (SG) (Gilead Sciences), sacituzumab tirumotecan (SKB264; sac-TMT) (Merck & Co.), datopotamab deruxtecan (Dato-dxd) (AstraZeneca/Daiichi Sankyo), SHR-A1921 (Jiangsu Hengrui Pharma), or ESG-401 (Shanghai Escugen Biotechnology). In some embodiments the ADC is a Nectin-4 ADC.

“Safe” treatment refers to a treatment without unacceptable toxicity in the human. In some embodiments, a “safe” treatment refers a treatment with “manageable toxicity profile” or “manageable adverse events profile.” In some embodiments, a “safe” treatment refers a treatment the patient can stay on treatment until progression.

“Safety signal” as used herein refers treatment related adverse event. A combination therapy without raising an additional safety signal compared to a group of monotherapies indicates that the combination therapy does not have additional TRAE compared to the combined TRAE of the group of monotherapies. In some embodiments, a combination therapy without raising an additional safety signal compared to a group of monotherapies indicates that the combination therapy does not have additional TRAE of grade ≥3 compared to the combined TRAE of grade ≥3 of the group of monotherapies.

“Standard of care,” as used herein refers to a preferred treatment regimen for a particular disease or indication, such as a treatment that a governmental regulatory agency has approved for the particular disease or indication.

“Targeted therapie(s)” refers to small molecule inhibitors, monoclonal antibodies, tumor agnostic treatments, and the like, in cancer treatment.

“Therapeutically effective amount” or “effective amount” is defined as an amount of compound/drug to treat the disease or disorders and achieve clinical efficacy.

“Tolerable” treatment refers to a treatment that does not cause a patient to discontinue the treatment due to TRAE.

“Treatment” (or grammatical variations thereof), refers to medical care that results in clinically meaningful benefit or clinically meaningful improvement in human patient(s) or human subject(s).

Treatment related adverse events (TRAEs), as used herein, refers adverse effects assessed by an investigator as at least possibly related to study treatment.

“Treatment-naïve,” as used herein, refers to not having received treatment, i.e., not having received treatment for locally advanced or metastatic urothelial carcinoma.

“Unacceptable toxicity” refers to toxicity that is considered unacceptable due to its severity and/or irreversibility. Such unacceptability may readily be determined by a treating physician or an investigator. In some embodiments, unacceptable toxicity refers to lifelong, significant health-adverse effects.

“Tolerable” treatment refers to a treatment that TRAEs do not cause a patient to discontinue the treatment.

“Urothelial carcinoma” refers cancer that begins in the urothelial cells. The majority of urothelial carcinoma begins in the bladder. As used herein, “urothelial carcinoma” may be used interchangeable with ““urothelial carcinoma” or “bladder cancer”.

### Methods of Treatment

In one aspect, provided herein is a method of treating locally advanced or metastatic urothelial carcinoma in a human subject, comprising administering to the subject a combination therapy which comprises: (i) a therapeutically effective amount of a Trop-2 antibody-drug conjugate (Trop-2 ADC) and (ii) a therapeutically effective amount of a Nectin-4 antibody-drug conjugate (Nectin-4 ADC). In some embodiments, the combination therapy does not comprise an additional therapy for locally advanced or metastatic urothelial carcinoma. In some embodiments the combination therapy does not include an anti-PD-1 antibody or anti-PD-L1 antibody. In some embodiments the combination therapy further comprises (iii) a therapeutically effective amount of an anti-PD-1 antibody or anti-PD-L1 antibody. In some embodiments, the combination therapy further comprises (iii) a therapeutically effective amount of an anti-PD-1 antibody. In some embodiments, the combination therapy further comprises (iii) a therapeutically effective amount of an anti-PD-L1 antibody.

In another aspect, provided herein is a Trop-2 ADC for use in a method of treating urothelial carcinoma in a human subject, said method comprising administering to the subject: (i) the Trop-2 ADC and (ii) a Nectin-4 ADC.

In another aspect, provided herein is a Nectin-4 ADC for use in a method of treating urothelial carcinoma in a human subject, said method comprising administering to the subject: (i) a Trop-2 ADC and (ii) the Nectin-4 ADC.

In another aspect, provided herein is a Trop-2 ADC and a Nectin-4 ADC for therapeutic use.

In another apect, provided herein is a Trop-2 ADC and a Nectin-4 ADC for use in a method of treating urothelial carcinoma in a human subject.

In another aspect, provided herein is an anti-PD-1 antibody or anti-PD-L1 antibody for use in a method of treating urothelial carcinoma in a human subject, said method comprising administering to the subject: (i) a Trop-2 ADC; (ii) a Nectin-4 ADC, and (iii) the anti-PD-1 antibody or anti-PD-L1 antibody.

In another aspect, provided herein is a method of treating relapsed or refractory locally advanced or metastatic urothelial carcinoma in a human subject, comprising administering to the subject a combination therapy which comprises: (i) a therapeutically effective amount of a Trop-2 ADC and (ii) a therapeutically effective amount of a Nectin-4 ADC; wherein the combination therapy does not comprise an anti-PD1 antibody or an anti-PD-L1 antibody, and wherein the subject has received one or more prior therapies for locally advanced or metastatic urothelial carcinoma.

In another aspect, provided herein is a method of treating locally advanced or metastatic urothelial carcinoma in a human subject, comprising administering to the subject a combination therapy which comprises: (i) a therapeutically effective amount of a Trop-2 ADC; (ii) a therapeutically effective amount of a Nectin-4 ADC, and (iii) a therapeutically effective amount of an anti-PD1 antibody or an anti-PD-L1 antibody; wherein the subject has not received a prior therapy for locally advanced or metastatic urothelial carcinoma.

In another aspect, provided herein is a method of safely treating locally advanced or metastatic urothelial carcinoma in a human subject, comprising administering to the subject a combination therapy which comprises: (i) a therapeutically effective amount of a Trop-2 ADC, and (ii) a therapeutically effective amount of a Nectin-4 ADC.

In another aspect, provided herein is a method of treating locally advanced or metastatic urothelial carcinoma that is well tolerated in a human subject, comprising administering to the subject a combination therapy which comprises: (i) a therapeutically effective amount of a Trop-2 ADC and (ii) a therapeutically effective amount of a Nectin-4 ADC.

In another aspect, provided herein is a method of improving clinical efficacy of a reference Trop-2 ADC monotherapy or a reference Nectin-4 ADC monotherapy for treating locally advanced or metastatic urothelial carcinoma in a human subject, comprising administering to the subject a combination therapy which comprises: (i) a therapeutically effective amount of a Trop-2 ADC and (ii) a therapeutically effective amount of a Nectin-4 ADC. In some embodiments, the methods provided herein improve the clinical efficacy of a reference Trop-2 ADC monotherapy. In some embodiments, the methods provided herein improve the clinical efficacy of a reference Nectin-4 ADC monotherapy.

In another aspect, provided herein is a method of improving clinical efficacy of a reference combination therapy comprising a reference Nectin-4 ADC and a reference anti-PD-1 antibody for treating locally advanced or metastatic urothelial carcinoma in a human subject, comprising administering to the subject a combination therapy which comprises: (i) a therapeutically effective amount of a Trop-2 ADC and (ii) a therapeutically effective amount of a Nectin-4 ADC.

In another aspect, provided herein is a method of treating locally advanced or metastatic urothelial carcinoma in a human subject using a combination therapy without raising an additional safety signal compared to a reference Trop-2 ADC monotherapy or a reference Nectin-4 ADC monotherapy, comprising administering to the subject the combination therapy which comprises: (i) a therapeutically effective amount of a Trop-2 ADC and (ii) a therapeutically effective amount of a Nectin-4 ADC.

In another aspect, provided herein is a method of treating locally advanced or metastatic urothelial carcinoma in a human subject using a combination therapy without raising an additional safety signal compared to a reference combination therapy comprising a reference Nectin-4 ADC and a reference anti-PD-1 antibody, comprising administering to the subject the combination therapy, which comprises: (i) a therapeutically effective amount of a Trop-2 ADC and (ii) a therapeutically effective amount of a Nectin-4 ADC.

In some embodiments, the reference Trop-2 ADC antibody or the reference Trop-2 monotherapy comprises sacituzumab govitecan (SG). In some embodiments the reference Trop-2 ADC monotherapy comprises administering SG at a dose of 10 mg/kg as an intravenous infusion on day 1 and day 8 of one or more 21-day treatment cycles.

In some embodiments, the reference Nectin-4 ADC antibody or the reference Nectin-4 monotherapy comprises enfortumab vedotin (EV). In some embodiments, the reference Nectin-4 monotherapy comprises administering EV at a dose of 1.25 mg/kg as an intravenous infusion on day 1, day 8, and day 15 of a 28-day treatment cycle. In some embodiments the reference combination therapy comprises administering EV at a dose of 1.25 mg/ml as an intravenous infusion on day 1 and day 8 of a 21-day treatment cycle and administering pembrolizumab at a dose of 200 mg on day 1 of the 21-day treatment cycle.

In some embodiments, the reference anti-PD-1 antibody is pembrolizumab.

### Trop-2 ADC

The Trop-2 ADCs that can be used in the methods provided herein comprise an anti-Trop2 antibody and a topoisomerase I inhibitor, which are optionally connected by a linker.

In some embodiments, the anti-Trop-2 antibody of the Trop-2 ADC is sacituzumab or datopotamab. In some embodiments the anti-Trop-2 antibody is sacituzumab. In some embodiments the anti-Trop-2 antibody is datopotamab.

In some embodiments, the topoisomerase I inhibitor in the Trop-2 ADC is SN38, exatecan, deruxtecan, or belotecan. In some embodiments the topoisomerase I inhibitor in the Trop-2 ADC is SN38. In some embodiments, the topoisomerase I inhibitor is exatecan. In some embodiments, the topoisomerase I inhibitor is deruxtecan. In some embodiments, the topoisomerase I inhibitor is belotecan.

In some embodiments, the Trop-2 ADC is sacituzumab govitecan (SG) (Gilead Sciences), sacituzumab tirumotecan (SKB264; sac-TMT) (Merck & Co.), datopotamab deruxtecan (Dato-dxd) (AstraZeneca/Daiichi Sankyo), SHR-A1921 (Jiangsu Hengrui Pharma), or ESG-401 (Shanghai Escugen Biotechnology). Trop-2 ADCs are described, for example, by Liu et al., Theranostics 2024; 14(9): 3674-3692. In some embodiments, the Trop-2 ADC is sac-TMT. In some embodiments, the Trop-2 ADC is Dato-dxd. In some embodiments the Trop-2 ADC is SG.

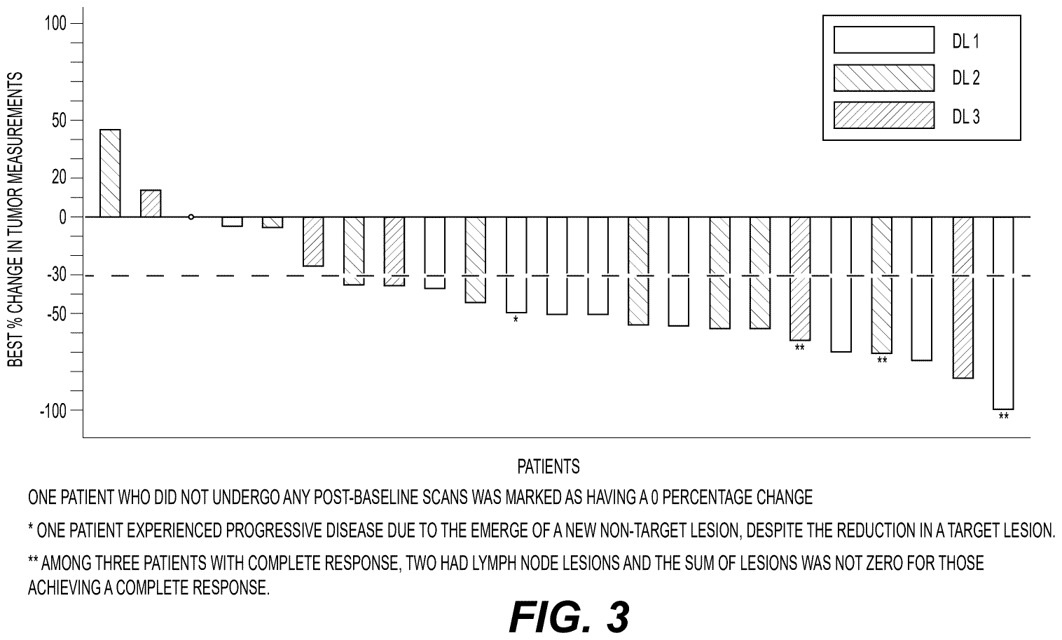

SG is a pharmaceutical composition comprising an antibody-drug conjugate (“ADC”) comprised of (1) a drug (“SN-38”), a topoisomerase 1 inhibitor that is an active metabolite of irinotecan; (2) a linker (“CL2A”); and (3) a humanized monoclonal antibody (“hRS7 IgGₖ” or “sacituzumab”). CL2A couples SN-38 to hRS7, which binds to Trop-2. In some embodiments, hRS7 is described, e.g., in WO2003074566, FIGS. 3 and 4, incorporated by reference in its entirety.

In some embodiments, SG is represented by Formula I as shown below.

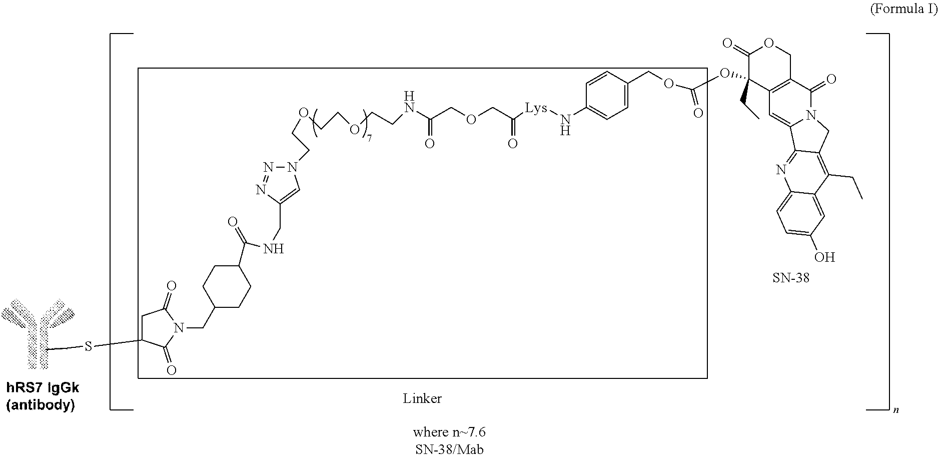

In some embodiments, the Trop-2 ADC comprises drug molecules linked to the antibody moieties in various stoichiometric molar ratios depending on the configuration of the antibody and, at least in part, the method used to effect configuration. In certain embodiments, the drug-antibody ratio (“DAR”) is about 7.6. In certain embodiments, the DAR is about 7.0 to about 8.0.

In certain embodiments, the hRS7 antibody in SG comprises the heavy chain as shown in SEQ ID NO: 14 and light chain as shown in SEQ ID NO: 13 (as shown in Table 1). In certain embodiments, the hRS7 antibody in SG comprises two heavy chains each having the sequence as shown in SEQ ID NO: 14, and two light chains each having the sequence as shown in SEQ ID NO: 13.

In certain embodiments, the hRS7 antibody in SG comprises light chain complementarity determining regions (CDRs) comprising a sequence of amino acids as set forth in SEQ ID NOs: 1, 2 and 3 and heavy chain CDRs comprising a sequence of amino acids as set forth in SEQ ID NOs: 4, 5 and 6.

TABLE 1
Amino Acid Sequences of hRS7 antibody

| SEQ ID | | |
|---|---|---|
| NO | Description | Amino Acid Sequence |
| 14 | hRS7 heavy | QVQLQQSGSELKKPGASVKVSCKASGYTFTNYGMNWVKQAPGQGLKWMGW |
| | chain | INTYTGEPTYTDDFKGRFAFSLDTSVSTAYLQISSLKADDTAVYFCARGG |
| | | FGSSYWYFDVWGQGSLVTVSSASTKGPSVFPLAPSSKSTSGGTAALGCLV |
| | | KDYFPEPVTVSWNSGALISGVHTFPAVLQSSGLYSLSSVVTVPSSSLGTQ |
| | | TYICNVNHKPSNTKVDKRVEPKSCDKTHTCPPCPAPELLGGPSVFLFPPK |
| | | PKDTLMISRTPEVTCVVVDVSHEDPEVKFNWYVDGVEVHNAKTKPREEQY |
| | | NSTYRVVSVLTVLHQDWLNGKEYKCKVSNKALPAPIEKTISKAKGQPREP |
| | | QVYTLPPSREEMTKNQVSLTCLVKGFYPSDIAVEWESNGQPENNYKTTPP |
| | | VLDSDGSFFLYSKLIVDKSRWQQGNVFSCSVMHEALHNHYTQKSLSLSPG |
| | | K |
| 13 | hRS7 light | DIQLTQSPSSLSASVGDRVSITCKASQDVSIAVAWYQQKPGKAPKLLIYS |
| | chain | ASYRYTGVPDRFSGSGSGTDFTLTISSLQPEDFAVYYCQQHYITPLIFGA |
| | | GTKVEIKRIVAAPSVFIFPPSDEQLKSGTASVVCLLNNFYPREAKVQWKV |
| | | DNALQSGNSQESVTEQDSKDSTYSLSSTLTLSKADYEKHKVYACEVTHQG |
| | | LSSPVTKSFNRGEC |
| 4 | HCDR1 | NYGMN |
| 5 | HCDR2 | WINTYTGEPTYTDDFKG |
| 6 | HCDR3 | GGFGSSYWYFDV |
| 1 | LCDR1 | KASQDVSIAVA |
| 2 | LCDR2 | SASYRYT |
| 3 | LCDR3 | QQHYITPLT |
| 17 | VH | QVQLQQSGSELKKPGASVKVSCKASGYTFTNYGMNWVKQAPGQGLKWMGW |
| | | INTYTGEPTYTDDFKGRFAFSLDTSVSTAYLQISSLKADDIAVYFCARGG |
| | | FGSSYWYFDVWGQGSLVTVSS |
| 18 | VL | DIQLTQSPSSLSASVGDRVSITCKASQDVSIAVAWYQQKPGKAPKLLIYS |
| | | ASYRYTGVPDRFSGSGSGTDFTLTISSLQPEDFAVYYCQQHYITPLTFGA |
| | | GTKVEIKR |

Exemplary Trop-2 ADCs that can be used in the methods provided herein are described, for example, in U.S. Pat. Nos. 7,999,083 and 9,028,833, which are hereby incorporated herein by reference in their entireties.

### Nectin-4 ADC

The Nectin-4 ADCs that can be used in the methods provided herein comprise an anti-Nectin-4 antibody and a microtubule inhibitor, which are optionally connected by a linker.

In some embodiments, the anti-Nectin-4 antibody of the Nectin-4 ADC is enfortumab.

In some embodiments the microtubule inhibitor in the Nectin-4 ADC is a maytansinoid or auristatin. In some embodiments the maytansinoid is maytansine, mertansine/emtansine (DM1) or raytansine/soravtansine (DM4). In some embodiments the auristatin is monomethyl auristatin E (MMAE) or monomethyl auristatin F (MMAF). In some embodiments, the microtubule inhibitor is monomethyl auristatin (MMAE).

In certain embodiments, the anti-Nectin-4 antibody in Nectin-4 ADC comprises the heavy chain as shown in SEQ ID NO: 16 and light chain as shown in SEQ ID NO: 15 (as shown in Table 2). In certain embodiments, the anti-Nectin-4 antibody in Nectin-4 ADC comprises two heavy chains each having the sequence as shown in SEQ ID NO: 16, and two light chains each having the sequence as shown in SEQ ID NO: 15.

In certain embodiments, the anti-Nectin-4 antibody in Nectin-4 ADC comprises the variable heavy chain as shown in SEQ ID NO: 19 and the variable light chain as shown in SEQ ID NO:20 (as shown in Table 2).

TABLE 2
Exemplary Nectin-4 Antibody Sequences

| SEQ ID | | |
|---|---|---|
| NO | Description | Amino Acid Sequence |
| 16 | Nectin-4 | EVQLVESGGGLVQPGGSLRLSCAASGFTFSSYNMNWVRQAPGKGL |
| | Antibody | EWVSYISSSSSTIYYADSVKGRFTISRDNAKNSLSLQMNSLRDEDTA |
| | heavy chain | VYYCARAYYYGMDVWGQGTTVTVSSASTKGPSVFPLAPSSKSTSG |
| | | GTAALGCLVKDYFPEPVTVSWNSGALTSGVHTFPAVLQSSGLYSLS |
| | | SVVTVPSSSLGTQTYICNVNHKPSNTKVDKRVEPKSCDKTHTCPPC |
| | | PAPELLGGPSVFLFPPKPKDTLMISRTPEVTCVVVDVSHEDPEVKEN |
| | | WYVDGVEVHNAKTKPREEQYNSTYRVVSVLTVLHQDWLNGKEY |
| | | KCKVSNKALPAPIEKTISKAKGQPREPQVYTLPPSREEMTKNQVSLT |
| | | CLVKGFYPSDIAVEWESNGQPENNYKTTPPVLDSDGSFFLYSKLTV |
| | | DKSRWQQGNVFSCSVMHEALHNHYTQKSLSLSPGK |
| 15 | Nectin-4 | DIQMTQSPSSVSASVGDRVTITCRASQGISGWLAWYQQKPGKAPKF |
| | Antibody | LIYAASTLQSGVPSRFSGSGSGTDFTLTISSLQPEDFATYYCQQANSF |
| | light chain | PPTFGGGTKVEIKRTVAAPSVFIFPPSDEQLKSGTASVVCLLNNFYP |
| | | REAKVQWKVDNALQSGNSQESVTEQDSKDSTYSLSSTLTLSKADY |
| | | EKHKVYACEVTHQGLSSPVTKSFNRGEC |
| 10 | HCDR1 | SYNMN |
| 11 | HCDR2 | YISSSSSTIYYADSVKG |
| 12 | HCDR3 | AYYYGMDV |
| 7 | LCDR1 | RASQGISGWLA |
| 8 | LCDR2 | AASTLQS |
| 9 | LCDR3 | QQANSFPPT |
| 19 | VH | EVQLVESGGGLVQPGGSLRLSCAASGFTFSSYNMNWVRQAPGKGL |
| | | EWVSYISSSSSTIYYADSVKGRFTISRDNAKNSLSLQMNSLRDEDTA |
| | | VYYCARAYYYGMDVWGQGTTVTVSS |
| 20 | VL | DIQMTQSPSSVSASVGDRVTITCRASQGISGWLAWYQQKPGKAPKF |
| | | LIYAASTLQSGVPSRFSGSGSGTDFTLTISSLQPEDFATYYCQQANSF |
| | | PPTFGGGTKVEIK |

In some embodiments of the methods provided herein, the Nectin-4 ADC is enfortumab vedotin (EV) (Seagen/Pfizer), CRB-701 (Corbus Pharmaceuticals), 9MW2821 (Mabwell), ADRX-0706 (Adcentrx), BT-8009 (BicycleTX), SYS-6002 (CSPC Megalith Biopharmaceutical), or ARC-101 (ARS Pharmaceuticals).

In some embodiments of the methods provided herein, the Nectin-4 ADC is enfortumab vedotin (EV) (Seagen/Pfizer), CRB-701 (Corbus Pharmaceuticals), 9MW2821 (Mabwell), or ADRX-0706 (Adcentrx).

In some embodiments, the Nectin-4 ADC is enfortumab vedotin (LV).

### Anti-PD-(L)1 Antibodies

The anti-PD(L)1 antibody used in the methods provided herein can be an anti-PD-1 antibody or anti-PD-1 antibody.

In some embodiments, combination therapies used in the methods provided herein comprise an anti-PD-1 antibody. In some embodiments, the anti-PD-1 antibody is nivolumab (also known as OPDIVO®, MDX-1106, BMS-936558, and ONO1152), zimberelimab, pidilizumab (also known as MDV9300, and CT-011), pembrolizumab (also known as KEYTRUDA®, MK-3477, SCH-900475, and lambrolizumab), BMS-936559, pidilizumab, PF-06801591, BGB-A317 (also known as tislelizumab), GLS-010 (WBP-3055), AK-103 (also known as HX-008), CS-1003, HLX-10, MGA-012, BI-754091, REGN-2810 (also known as cemiplimab), AGEN-2034 (also known as balstilimab), JS-001 (also known as toripalmilab), JNJ-63723283, genolimzumab (also known as CBT-501), LZM-009, BCD-100, LY-3300054, SHR-1201, SHR-1210 (also known as camrelizumab), Sym-021, ABBV-181, AK-105, PD1-PIK, or BAT-1306. In some embodiments, the anti-PD-1 antibody is pembrolizumab, nivolumab, cemiplimab, pidilizumab, spartalizumab, sasanlimab, tislelizumab, retifanlimab, balstilimab, toripalimab, cetrelimab, genolimzumab, prolgolimab, camrelizumab, dostarlimab, sintilimab, or zimberelimab. In some embodiments the anti-PD-1 antibody is pembrolizumab. In some embodiments, the anti-PD-1 antibody is zimberelimab.

In some embodiments, the combination therapies comprise an anti-PD-L1 antibody. In some embodiments, the anti-PD-L1 antibody is BMS-936559, atezolizumab (also known as MPDL3280A), durvalumab (also known as MEDI4736), avelumab, CK-301 (also known as MSB0010718C), MEDI0680, CX-072, CBT-502, PDR-001 (also known as spartalizumab), TSR-042 (also known as dostarimab), JTX-4014, BGB-A333, SHR-1316, CS-1001 (also known as WBP-3155), KN-035, IBI-308 (also known as sintilimab), HLX-20, KL-A167, STI-A1014, STI-A1015 (also known as IMC-001), BCD-135, FAZ-053, TQB-2450, or MDX1105-01. In some embodiments, the anti-PD-L1 antibody is atezolizumab, avelumab, durvalumab, cosibelimab, lodapolimab, budigalimab, avelumab, or envafolimab.

Examples of monoclonal antibodies (mAbs) that bind to human PD-1, useful in the treatment methods and uses of the invention, are described in U.S. Pat. Nos. 7,521,051, 8,008,449, and 8,354,509. Specific anti-human PD-1 mAbs useful as a PD-1 antagonist in the treatment methods of the present disclosure include: pembrolizumab (formerly known as MK-3475, SCH 900475 and lambrolizumab), a humanized IgG4 mAb with the structure described in WHO Drug Information, Vol. 27, No. 2, pages 161-162 (2013), and the humanized antibodies h409A11, h409A16 and h409A17, which are described in WO 2008/156712.

In some embodiments of the methods of the present disclosure, the anti-PD-1 antibody, or antigen binding fragment thereof, comprises: (a) light chain CDRs comprising a sequence of amino acids as set forth in SEQ ID NOs: 21, 22 and 23, and (b) heavy chain CDRs comprising a sequence of amino acids as set forth in SEQ ID NOs: 26, 27 and 28.

In some embodiments, the anti-PD-1 antibody or antigen binding fragment thereof is a human antibody. In other embodiments, the anti-PD-1 antibody or antigen binding fragment thereof is a humanized antibody. In other embodiments, the anti-PD-1 antibody or antigen binding fragment thereof is a chimeric antibody. In specific embodiments, the anti-PD-1 antibody or antigen binding fragment thereof is a monoclonal antibody.

In other embodiments of the treatment methods of the present disclosure, the anti-PD-1 antibody, or antigen binding fragment thereof, specifically binds to human PD-1 and comprises (a) a heavy chain variable region comprising an amino acid sequence as set forth in SEQ ID NO:29, or a variant thereof, and (b) a light chain variable region comprising an amino acid sequence selected from the group consisting of SEQ ID NO:24.

A variant of a heavy chain variable region sequence or full-length heavy chain sequence is identical to the reference sequence except having up to 17 conservative amino acid substitutions in the framework region (i.e., outside of the CDRs), and preferably has less than ten, nine, eight, seven, six or five conservative amino acid substitutions in the framework region. A variant of a light chain variable region sequence or full-length light chain sequence is identical to the reference sequence except having up to five conservative amino acid substitutions in the framework region (i.e., outside of the CDRs), and preferably has less than four, three or two conservative amino acid substitution in the framework region.

In another embodiment of the treatment methods of the present disclosure, the anti-PD-1 antibody or antigen-binding fragment thereof is a monoclonal antibody which specifically binds to human PD-1 and comprises (a) a heavy chain comprising or consisting of a sequence of amino acids as set forth in SEQ ID NO:30, or a variant thereof; and (b) a light chain comprising or consisting of a sequence of amino acids as set forth in SEQ ID NO:25, or a variant thereof.

In yet another embodiment of the treatment methods of the present disclosure, the anti-PD-1 antibody or antigen-binding fragment thereof is a monoclonal antibody which specifically binds to human PD-1 and comprises (a) a heavy chain comprising or consisting of a sequence of amino acids as set forth in SEQ ID NO:30 and (b) a light chain comprising or consisting of a sequence of amino acids as set forth in SEQ ID NO:25.

TABLE 3
Exemplary PD-1 Antibody Sequences

| | | |
|---|---|---|
| Antibody | | SEQ |
| Feature | Amino Acid Sequence | ID NO |

Pembrolizumab Light Chain

| | | |
|---|---|---|
| CDR1 | RASKGVSTSGYSYLH | 21 |
| CDR2 | LASYLES | 22 |
| CDR3 | QHSRDLPLT | 23 |
| Variable | EIVLTQSPATLSLSPGERATLSCRASKGVSTSGYSYLHWYQ | 24 |
| Region | QKPGQAPRLLIYLASYLESGVPARFSGSGSGTDFTLTISSL | |
| | EPEDFAVYYCQHSRDLPLTFGGGTKVEIK | |
| Light Chain | EIVLTQSPATLSLSPGERATLSCRASKGVSTSGYSYLHWYQ | 25 |
| | QKPGQAPRLLIYLASYLESGVPARFSGSGSGTDFTLTISSL | |
| | EPEDFAVYYCQHSRDLPLTFGGGTKVEIKRTVAAPSVFIFP | |
| | PSDEQLKSGTASVVCLLNNFYPREAKVQWKVDNALQSGNSQ | |
| | ESVTEQDSKDSTYSLSSTLTLSKADYEKHKVYACEVTHQGL | |
| | SSPVTKSFNRGEC | |

Pembrolizumab Heavy Chain

| | | |
|---|---|---|
| CDR1 | NYYMY | 26 |
| CDR2 | GINPSNGGTNFNEKFKN | 27 |
| CDR3 | RDYRFDMGFDY | 28 |
| Variable | QVQLVQSGVEVKKPGASVKVSCKASGYTFTNYYMYWVR | 29 |
| Region | QAPGQGLEWMGGINPSNGGTNFNEKFKNRVTLTTDSSTT | |
| | TAYMELKSLQFDDTAVYYCARRDYRFDMGFDYWGQGTT | |
| | VTVSS | |
| Heavy | QVQLVQSGVEVKKPGASVKVSCKASGYTFTNYYMYWVRQ | 30 |
| Chain | APGQGLEWMGGINPSNGGTNFNEKFKNRVTLTTDSSTTT | |
| | AYMELKSLQFDDTAVYYCARRDYRFDMGFDYWGQGTTVT | |
| | VSSASTKGPSVFPLAPCSRSTSESTAALGCLVKDYFPEP | |
| | VTVSWNSGALTSGVHTFPAVLQSSGLYSLSSVVTVPSSS | |
| | LGTKTYTCNVDHKPSNTKVDKRVESKYGPPCPPCPAPEF | |
| | LGGPSVFLFPPKPKDTLMISRTPEVTCVVVDVSQEDPEV | |
| | QFNWYVDGVEVHNAKTKPREEQFNSTYRVVSVLTVLHQD | |
| | WLNGKEYKCKVSNKGLPSSIEKTISKAKGQPREPQVYTL | |
| | PPSQEEMTKNQVSLTCLVKGFYPSDIAVEWESNGQPENN | |
| | YKTTPPVLDSDGSFFLYSRLTVDKSRWQEGNVFSCSVMH | |
| | EALHNHYTQKSLSLSLGK | |

In one embodiment, the anti-PD-1 antibody or antigen-binding fragment thereof comprises a heavy chain constant region, e.g., a human constant region, such as g1, g2, g3, or g4 human heavy chain constant region or a variant thereof. In another embodiment, the anti-PD-1 antibody or antigen-binding fragment thereof comprises a light chain constant region, e.g., a human light chain constant region, such as lambda or kappa human light chain region or a variant thereof. By way of example, and not limitation, the human heavy chain constant region can be g4 and the human light chain constant region can be kappa. In an alternative embodiment, the Fc region of the antibody is g4 with a Ser228Pro mutation (Schuurmnan, J et. al., Mol. Immunol. 38: 1-8, 2001). In some embodiments, different constant domains may be appended to humanized VL and VH regions derived from the CDRs provided herein. For example, if a particular intended use of an antibody (or fragment) of the present invention were to call for altered effector functions, a heavy chain constant domain other than human IgG1 may be used, or hybrid IgG1/IgG4 may be utilized. Although human IgG1 antibodies provide for long half-life and for effector functions, such as complement activation and antibody-dependent cellular cytotoxicity, such activities may not be desirable for all uses of the antibody. In such instances a human IgG4 constant domain, for example, may be used. The present invention includes the use of anti-PD-1 antibodies or antigen-binding fragments thereof which comprise an IgG4 constant domain. In one embodiment, the IgG4 constant domain can differ from the native human IgG4 constant domain (Swiss-Prot Accession No. P01861.1) at a position corresponding to position 228 in the EU system and position 241 in the KABAT system, where the native Ser108 is replaced with Pro, in order to prevent a potential inter-chain disulfide bond between Cys106 and Cys109 (corresponding to positions Cys 226 and Cys 229 in the EU system and positions Cys 239 and Cys 242 in the KABAT system) that could interfere with proper intra-chain disulfide bond formation. See Angal et al. (1993) Mol. Imunol. 30:105. In other instances, a modified IgG1 constant domain which has been modified to increase half-life or reduce effector function can be used.

In another embodiment, the anti-PD-1 antibody or antigen binding fragment thereof has a variable light domain and/or a variable heavy domain with at least 95%, 90%, 85%, 80%, 75% or 50% sequence identity to one of the variable light domains or variable heavy domains described above, and exhibits specific binding to PD-1. In another embodiment of the methods of treatment of the invention, the anti-PD-1 antibody or antigen binding fragment thereof comprises variable light and variable heavy domains having up to 1, 2, 3, 4, or 5 or more amino acid substitutions, and exhibits specific binding to PD-1.

In some embodiments, the anti-PD-1 antibody is zimberelimab.

### Dosing Regimens

The Trop-2 ADC, the Nectin-4 ADC, and, optionally, the anti-PD(L)1 antibody (anti-PD-1 antibody or anti-PD-L1 antibody) in the combination therapies disclosed herein are each independently administered to a human subject. In some embodiments, the Trop-2 ADC, the Nectin-4 ADC, and, optionally, the anti-PD(L)-1 antibody are each administered as intravenous infusions. In some embodiments, administration of the Trop-2 ADC, the Nectin-4 ADC, and, optionally, the anti-PD(L)-1 antibody in the combination therapies disclosed herein occurs in treatment cycles. In some embodiments the treatment cycles are continuous. In some embodiments, at least 10, 11, 12, 13, 14, 15, 16, 17, 18, 19, or 20 continuous treatment cycles of combination therapies are administered to the human subject. In some embodiments, the combination therapy is administered until disease progression or unacceptable toxicity. In some embodiments, administration of the Trop-2 ADC, the Nectin-4 ADC, and, optionally, the anti-PD(L)-1 antibody continues until cancer progression or unacceptable toxicity is observed in the human subject. In some embodiments, the treatment cycles are each 21-day treatment cycles. In some embodiments, the Trop-2 ADC and the Nectin-4 ADC are each independently administered on day 1 and day 8 of continuous 21-day treatment cycles. In some embodiments, the anti-PD(L)-1 antibody is further independently administered on day 1 of continuous 21-day treatment cycles. In some embodiments, the anti-PD(L)1 antibody is further independently administered on day 1 or every other cycle of the continuous 21-day treatment cycles.

In some embodiments, the Trop-2 ADC is administered as an intravenous infusion on day 1 and day 8 of one or more 21-day treatment cycles. In some embodiments, the Trop-2 ADC is administered at a dose of from 5 mg/kg to 10 mg/kg as an intravenous infusion on day 1 and day 8 of one or more 21-day treatment cycles. In some embodiments, the Trop-2 ADC is administered at a dose of 5 mg/kg, 7.5 mg/kg, 8 mg/kg, or 10 mg/kg. In some embodiments, the Trop-2 ADC is administered at a dose of 5 mg/kg as an intravenous infusion on day 1 and day 8 of a 21-day treatment cycle. In some embodiments, the Trop-2 ADC is administered at a dose of 7.5 mg/kg as an intravenous infusion on day 1 and day 8 of a 21-day treatment cycle. In some embodiments, the Trop-2 ADC is administered at a dose of 8 mg/kg as an intravenous infusion on day 1 and day 8 of a 21-day treatment cycle. In some embodiments, the Trop-2 ADC is administered at a dose of 10 mg/kg as an intravenous infusion on day 1 and day 8 of a 21-day treatment cycle. In some embodiments, the Trop-2 ADC is SG. In some embodiments, the Trop-2 ADC is Dato-dxd. In some embodiments, the Trop-2 ADC is sac-TMT.

In some embodiments, the Nectin-4 ADC is administered as an intravenous infusion on day 1 and day 8 of a 21-day treatment cycle. In some embodiments, the Nectin-4 ADC is administered at a dose of from 0.5 mg/kg to 1.25 mg/kg as an intravenous infusion on day 1 and day 8 of a 21-day treatment cycle. In some embodiments, the Nectin-4 ADC is administered at a dose of 0.5 mg/kg as an intravenous infusion on day 1 and day 8 of a 21-day treatment cycle. In some embodiments, the Nectin-4 ADC is administered at a dose of 0.75 mg/kg as an intravenous infusion on day 1 and day 8 of a 21-day treatment cycle. In some embodiments, the Nectin-4 ADC is administered at a dose of 1 mg/kg as an intravenous infusion on day 1 and day 8 of one or more 21-day treatment cycles. In some embodiments, the Nectin-4 ADC is administered at a dose of 1.25 mg/kg as an intravenous infusion on day 1 and day 8 of one or more 21-day treatment cycles. In some embodiments, the Nectin-4 ADC is EV.

In some embodiments, the anti-PD(L)1 antibody is an anti-PD-1 antibody. In some embodiments, the anti-PD-1 antibody is administered at a dose of 200 mg as an intravenous infusion on day 1 of each 21-day treatment cycle. In some embodiments, the anti-PD-1 antibody is administered at a dose of 400 mg as an intravenous infusion on day 1 of each second dosing cycle. In some embodiments the anti-PD-1 antibody is administered at a dose of 360 mg as an intravenous infusion on day 1 of each 21-day dosing cycle.

In certain embodiments, in the combination therapy disclosed herein, SG is administered as an intravenous infusion at a dose of from 5 mg/kg to 10 mg/kg on days 1 and 8 of continuous 21-day treatment cycles, and EV is administered as an intravenous infusion at a dose of from 0.5 mg/kg to 1.25 mg/kg on days 1 and 8 of continuous 21-day treatment cycles. In certain embodiments, SG is administered until disease progression or unacceptable toxicity. In certain embodiments, EV is administered until disease progression or unacceptable toxicity. In certain embodiments, SG and EV are administered until disease progression or unacceptable toxicity. In certain embodiments, SG and EV are administered for at least 10, 11, 12, 13, 14, 15, 16, 17, 18, 19, or 20 continuous 21-day treatment cycles to the human subject.

In certain embodiments, in the combination therapy disclosed herein, SG is administered as an intravenous infusion at a dose of 5 mg/kg, 7.5 mg/kg, 8 mg/kg, or 10 mg/kg on days 1 and 8 of continuous 21-day treatment cycles, and EV is administered as an intravenous infusion at a dose of 0.5 mg/kg, 0.75 mg/kg, 1 mg/kg, or 1.25 mg/kg on days 1 and 8 of continuous 21-day treatment cycles.

In certain embodiments, in the combination therapy disclosed herein, SG is administered as an intravenous infusion at a dose of 7.5 mg/kg or 5 mg/kg on days 1 and 8 of continuous 21-day treatment cycles, and EV is administered as an intravenous infusion at a dose of 0.5 mg/kg, 0.75 mg/kg, 1 mg/kg, or 1.25 mg/kg on days 1 and 8 of continuous 21-day treatment cycles.

In certain embodiments, in the combination therapy disclosed herein, SG is administered as an intravenous infusion at a dose of 5 mg/kg on days 1 and 8 of continuous 21-day treatment cycles, and EV is administered as an intravenous infusion at a dose of 0.5 mg/kg on days 1 and 8 of continuous 21-day treatment cycles.

In certain embodiments, in the combination therapy disclosed herein, SG is administered as an intravenous infusion at a dose of 5 mg/kg on days 1 and 8 of continuous 21-day treatment cycles, and EV is administered as an intravenous infusion at a dose of 0.75 mg/kg on days 1 and 8 of continuous 21-day treatment cycles.

In certain embodiments, in the combination therapy disclosed herein, SG is administered as an intravenous infusion at a dose of 5 mg/kg on days 1 and 8 of continuous 21-day treatment cycles, and EV is administered as an intravenous infusion at a dose of 1 mg/kg on days 1 and 8 of continuous 21-day treatment cycles.

In certain embodiments, in the combination therapy disclosed herein, SG is administered as an intravenous infusion at a dose of 5 mg/kg on days 1 and 8 of continuous 21-day treatment cycles, and EV is administered as an intravenous infusion at a dose of 1.25 mg/kg on days 1 and 8 of continuous 21-day treatment cycles.

In certain embodiments, in the combination therapy disclosed herein, SG is administered as an intravenous infusion at a dose of 7.5 mg/kg on days 1 and 8 of continuous 21-day treatment cycles, and EV is administered as an intravenous infusion at a dose of 0.5 mg/kg on days 1 and 8 of continuous 21-day treatment cycles.

In certain embodiments, in the combination therapy disclosed herein, SG is administered as an intravenous infusion at a dose of 7.5 mg/kg on days 1 and 8 of continuous 21-day treatment cycles, and EV is administered as an intravenous infusion at a dose of 0.75 mg/kg on days 1 and 8 of continuous 21-day treatment cycles.

In certain embodiments, in the combination therapy disclosed herein, SG is administered as an intravenous infusion at a dose of 7.5 mg/kg on days 1 and 8 of continuous 21-day treatment cycles, and EV is administered as an intravenous infusion at a dose of 1 mg/kg on days 1 and 8 of continuous 21-day treatment cycles.

In certain embodiments, in the combination therapy disclosed herein, SG is administered as an intravenous infusion at a dose of 7.5 mg/kg on days 1 and 8 of continuous 21-day treatment cycles, and EV is administered as an intravenous infusion at a dose of 1.25 mg/kg on days 1 and 8 of continuous 21-day treatment cycles.

In certain embodiments, in the combination therapy disclosed herein, SG is administered as an intravenous infusion at a dose of 8 mg/kg on days 1 and 8 of continuous 21-day treatment cycles, and EV is administered as an intravenous infusion at a dose of 0.5 mg/kg on days 1 and 8 of continuous 21-day treatment cycles.

In certain embodiments, in the combination therapy disclosed herein, SG is administered as an intravenous infusion at a dose of 8 mg/kg on days 1 and 8 of continuous 21-day treatment cycles, and EV is administered as an intravenous infusion at a dose of 0.75 mg/kg on days 1 and 8 of continuous 21-day treatment cycles.

In certain embodiments, in the combination therapy disclosed herein, SG is administered as an intravenous infusion at a dose of 8 mg/kg on days 1 and 8 of continuous 21-day treatment cycles, and EV is administered as an intravenous infusion at a dose of 1 mg/kg on days 1 and 8 of continuous 21-day treatment cycles.

In certain embodiments, in the combination therapy disclosed herein, SG is administered as an intravenous infusion at a dose of 8 mg/kg on days 1 and 8 of continuous 21-day treatment cycles, and EV is administered as an intravenous infusion at a dose of 1.25 mg/kg on days 1 and 8 of continuous 21-day treatment cycles.

In certain embodiments, in the combination therapy disclosed herein, SG is administered as an intravenous infusion at a dose of 10 mg/kg on days 1 and 8 of continuous 21-day treatment cycles, and EV is administered as an intravenous infusion at a dose of 0.5 mg/kg on days 1 and 8 of continuous 21-day treatment cycles.

In certain embodiments, in the combination therapy disclosed herein, SG is administered as an intravenous infusion at a dose of 10 mg/kg on days 1 and 8 of continuous 21-day treatment cycles, and EV is administered as an intravenous infusion at a dose of 0.75 mg/kg on days 1 and 8 of continuous 21-day treatment cycles.

In certain embodiments, in the combination therapy disclosed herein, SG is administered as an intravenous infusion at a dose of 10 mg/kg on days 1 and 8 of continuous 21-day treatment cycles, and EV is administered as an intravenous infusion at a dose of 1 mg/kg on days 1 and 8 of continuous 21-day treatment cycles.

In certain embodiments, in the combination therapy disclosed herein, SG is administered as an intravenous infusion at a dose of 10 mg/kg on days 1 and 8 of continuous 21-day treatment cycles, and EV is administered as an intravenous infusion at a dose of 1.25 mg/kg on days 1 and 8 of continuous 21-day treatment cycles.

In certain embodiments, in the combination therapy disclosed herein, SG is administered as an intravenous infusion at a dose of 7.5 mg/kg on days 1 and 8 of first 1 to 3 continuous 21-day treatment cycles, and EV is administered as an intravenous infusion at a dose of 1.25 mg/kg on days 1 and 8 of first 1 to 3 continuous 21-day treatment cycles; subsequently, SG is administered as an intravenous infusion at a dose of 5 mg/kg on days 1 and 8 of following 1-3 continuous 21-day treatment cycles, and EV is administered as an intravenous infusion at a dose of 1 mg/kg on days 1 and 8 of following 1 to 3 continuous 21-day treatment cycles. In certain embodiments, the combination therapy disclosed herein further comprising subsequently administering SG an intravenous infusion at a dose of 5 mg/kg on days 1 and 8 of following 1-3 continuous 21-day treatment cycles, and administering EV as an intravenous infusion at a dose of 0.75 mg/kg on days 1 and 8 of following 1 to 3 continuous 21-day treatment cycles. In certain embodiments, the combination therapy disclosed herein further comprising subsequently administering SG an intravenous infusion at a dose of 5 mg/kg on days 1 and 8 of following 1-3 continuous 21-day treatment cycles, and administering EV as an intravenous infusion at a dose of 0.5 mg/kg on days 1 and 8 of following 1 to 3 continuous 21-day treatment cycles.

In certain embodiments, the combination therapy disclosed herein does not include a PD-(L)1 inhibitor. In certain embodiments, the combination therapy disclosed herein does not include an anti-PD(L)1 antibody (anti-PD-1 antibody or anti-PD-L1 antibody). In certain embodiments, the combination therapy disclosed herein further includes a PD-(L)1 inhibitor (anti-PD-1 antibody or anti-PD-L1 antibody). In certain embodiments, the combination therapy includes an anti-PD-1 antibody. In certain embodiments, the anti-PD-1 antibody is pembrolizumab, nivolumab, cemiplimab, pidilizumab, spartalizumab, sasanlimab, tislelizumab, retifanlimab, balstilimab, toripalimab, cetrelimab, genolimzumab, prolgolimab, camrelizumab, dostarlimab, sintilimab, or zimberelimab. In certain embodiments, the combination therapy includes an anti-PD-L1 antibody. In certain embodiments, the anti-PD-L1 antibody is atezolizumab, avelumab, durvalumab, cosibelimab, lodapolimab, budigalimab, avelumab, or envafolimab.

In certain embodiments, the combination therapy disclosed herein further includes pembrolizumab, and pembrolizumab is administered a dose level of 200 mg on day 1 of the continuous 21-day treatment cycles. In certain embodiments, the combination therapy disclosed herein further includes pembrolizumab, and pembrolizumab is administered a dose level of 400 mg on day 1 of continuous 42-day treatment cycles (day 1 of every other of the continuous 21-day treatment cycles).

In certain embodiments, the combination therapy disclosed herein includes zimberelimab, and zimberelimab is administered as an intravenous infusion on day 1 of continuous 21-day treatment cycles. In some embodiments, zimberelimab is administered at one or more doses in the range of 300 mg to 400 mg. In some embodiments, zimberelimab is administered at dose of 360 mg. In some embodiments, zimberelimab is administered on day 1 of a 21-day cycle.

In certain embodiments of the combination therapy, EV and SG are administered in the order of EV, then SG.

In certain embodiments, EV, pembrolizumab, and SG are administered in the order of EV, pembrolizumab, then SG. In certain embodiments, pembrolizumab is administered 15 to 60 minutes after completing EV administrations. In certain embodiments, pembrolizumab is administered 15 minutes after completing EV administrations. In certain embodiments, pembrolizumab is administered 30 minutes after completing EV administrations. In certain embodiments, pembrolizumab is administered 60 minutes after completing EV administrations.

### Premedication

In certain embodiments, patients treated with the methods disclosed herein have received premedication comprising antipyretics, H1 blocker, and H2 blocker. In certain embodiments, the antipyretics is acetaminophen; the H1 blocker is Benadryl or Loratidine; and the H2 blocker is Pepcid. In certain embodiments, patients treated with the methods disclosed herein have received acetaminophen 650 mg PO; Benadryl 25 mg PO/IV or Loratidine 10 mg PO; and H2 blocker Pepcid 20 mg PO/IV.

In certain embodiments, patients treated with the methods disclosed herein have received antiemetic premedication comprising a 5-HT3 inhibitor, an NK1-receptor antagonist, and dexamethasone. In certain embodiments, the 5-HT3 inhibitor is ondansetron or palonosetron; and the NK1-receptor antagonist is fosaprepitant or aprepitant.

In certain embodiments, patients treated with the methods disclosed herein have received growth factors. In certain embodiments, patients treated with the methods disclosed herein have received prophylactic granulocyte colony stimulating factor.

In certain embodiments, the premedication is administered approximately 30 minutes before each SG administration.

### Patient Populations

In some embodiments, the methods provided herein can be used to treat cancer patients. In some embodiments, the cancer patients are human cancer patients. In some embodiments, the cancer patients are human patients with bladder cancer. In some embodiments, the bladder cancer is urothelial carcinoma.

In certain embodiments, patients treated with the methods disclosed herein have relapsed or refractory locally advanced or metastatic urothelial carcinoma. In certain embodiments, the urothelial carcinoma is locally advanced. In certain embodiments, the urothelial carcinoma is metastatic. In certain embodiments, the urothelial carcinoma is treatment naïve. In certain embodiments, the urothelial carcinoma is chemotherapy naïve.

In certain embodiments, patients treated with the methods disclosed herein are platinum-ineligible. In certain embodiments, patients treated with the methods disclosed herein are cisplatin-ineligible. In certain embodiments, patients treated with the methods disclosed herein are carboplatin-ineligible.

In certain embodiments, the methods disclosed herein are used to treat first line urothelial carcinoma. In certain embodiments, the methods disclosed herein are used to treat first line locally advanced or metastatic urothelial carcinoma. In certain embodiments, the methods disclosed herein are used to treat first line metastatic urothelial carcinoma.

In certain embodiments, patients treated with the methods disclosed herein have treatment naïve urothelial carcinoma. In certain embodiments, patients treated with the methods disclosed herein have chemotherapy naive urothelial carcinoma. In certain embodiments, patients treated with the methods disclosed herein have not had prior therapy for urothelial carcinoma. In certain embodiments, patients treated with the methods disclosed herein have not had prior systemic therapy for urothelial carcinoma. In certain embodiments, the patients have not had prior systemic therapy for metastatic urothelial carcinoma. In certain embodiments, patients treated with the methods disclosed herein are ineligible for or refusing cisplatin based therapy. In certain embodiments, the patients have received adjuvant or neoadjuvant therapy that was completed at least 6 months prior to development of metastatic disease. In certain embodiments, the patients have not received adjuvant or neoadjuvant therapy that was completed at least 6 months prior to development of metastatic disease. In certain embodiments, the patient having locally advanced or metastatic urothelial carcinoma has not had prior therapy for locally advanced or metastatic urothelial carcinoma prior to the combination therapy of SG and EV as disclosed herein. In certain embodiments, the patient has not had prior targeted therapy (either approved or investigational) for urothelial carcinoma.

In certain embodiments, the methods disclosed herein do not comprise concomitant administration of an additional systemic therapy. In certain embodiments, the methods disclosed herein do not comprise concomitant administration of an additional anti-cancer agent. In certain embodiments, the methods disclosed herein do not comprise concomitant administration of chemotherapy. In certain embodiments, the methods disclosed herein do not comprise concomitant administration of a platinum-based chemotherapy. In certain embodiments, the methods disclosed herein do not comprise concomitant administration of a PD-(L)1 inhibitor (e.g., an anti-PD(L)-1 antibody). In certain embodiments, the methods disclosed herein do not comprise concomitant administration of pembrolizumab. In certain embodiments, the methods disclosed herein do not comprise concomitant administration of zimberelimab. In certain embodiments, the methods disclosed herein do not comprise concomitant administration of platinum-based chemotherapy.

In certain embodiments, the patients treated with the methods disclosed herein have had a prior systemic therapy for metastatic urothelial carcinoma. In certain embodiments, the patients treated with the methods disclosed herein have received prior platinum-based therapy for metastatic urothelial carcinoma, the metastatic urothelial carcinoma has progressed during or after platinum-based therapy. In certain embodiments, the patients treated with the methods disclosed herein have received prior immunotherapy for metastatic urothelial carcinoma, the metastatic urothelial carcinoma has progressed during or after immunotherapy. In certain embodiments, the patients treated with the methods disclosed herein have received prior platinum-based therapy and immunotherapy for metastatic urothelial carcinoma, the metastatic urothelial carcinoma has progressed during or after platinum-based therapy and immunotherapy.

In certain embodiments, the patients treated with the methods disclosed herein have received prophylactic GCSF.

### Safety

In certain embodiments, the combination therapy disclosed herein is safe. In certain embodiments, the combination therapy including a Trop-2 ADC and a Nectin-4 ADC is safe. In certain embodiments, the combination therapy including SG and EV is safe. In some embodiments, a therapy is safe indicates that the therapy has manageable toxicity profile or manageable adverse events profile. In some embodiments, a patient can stay on the combination therapy including SG and EV until progression.

In certain embodiments, the present disclosure provides a method of treating relapsed or refractory locally advanced or metastatic urothelial carcinoma in a subject, including administering to the subject a combination therapy which includes:
a therapeutically effective amount of a Trop-2 ADC; and
a therapeutically effective amount of a Nectin-4 ADC;
wherein the method is safe.

In certain embodiments, the present disclosure provides a method of treating relapsed or refractory locally advanced or metastatic urothelial carcinoma in a subject, including administering to the subject a combination therapy which includes:
a therapeutically effective amount of a Trop-2 ADC; and
a therapeutically effective amount of a Nectin-4 ADC)
wherein the method is tolerable.

In certain embodiments, the present disclosure provides a method of treating relapsed or refractory locally advanced or metastatic urothelial carcinoma in a subject, including administering to the subject a combination therapy including SG and EV, wherein the method is safe.

In certain embodiments, the present disclosure provides a method of treating relapsed or refractory locally advanced or metastatic urothelial carcinoma in a subject, including administering to the subject a combination therapy including SG and EV, wherein the method is tolerable.

In certain embodiments, the combination therapy disclosed herein is tolerable. In certain embodiments, the combination therapy including a Trop-2 ADC and a Nectin-4 ADC is tolerable. In certain embodiments, the combination therapy including SG and EV is tolerable.

In certain embodiments, when the combination therapy disclosed herein is used to treat a population of patients, less than about 20% of the patients have serious treatment related adverse events (TRAEs). In certain embodiments, the combination therapy including a Trop-2 ADC and a Nectin-4 ADC is used to treat a population of patients, less than about 20% of the patients have serious TRAE. In certain embodiments, the combination therapy including SG and EV is used to treat a population of patients, less than about 20% of the patients have serious TRAE. In certain embodiments, when the combination therapy disclosed herein is used to treat a patient, the patient does not have serious treatment related adverse events (TRAEs). In certain embodiments, the combination therapy including a Trop-2 ADC and a Nectin-4 ADC is used to treat a patient, the patient does not have serious TRAE. In certain embodiments, the combination therapy including SG and EV is used to treat a patient, the patient does not have serious TRAE.

In certain embodiments, the combination therapy disclosed herein achieves less than about 60% TRAE of grade ≥3. In certain embodiments, the combination therapy including a Trop-2 ADC and a Nectin-4 ADC achieves less than about 60% TRAE of grade ≥3. In certain embodiments, the combination therapy including SG and EV achieves less than about 60% TRAE of grade ≥3. In certain embodiments, the combination therapy disclosed herein achieves less than about 70% TRAE of grade ≥3. In certain embodiments, the combination therapy disclosed herein achieves less than about 80% TRAE of grade ≥3. In certain embodiments, the combination therapy disclosed herein is used to treat a patient, the patient does not have TRAE of grade ≥3. In certain embodiments, the combination therapy including a Trop-2 ADC and a Nectin-4 ADC is used to treat a patient, the patient does not have TRAE of grade ≥3. In certain embodiments, the combination therapy including SG and EV is used to treat a patient, the patient does not have TRAE of grade ≥3.

In certain embodiments, the present disclosure provides a method of treating relapsed or refractory locally advanced or metastatic urothelial carcinoma in a population of subjects/patients, including administering to each subject/patient a combination therapy including a therapeutically effective amount of SG and a therapeutically effective amount of EV. In certain embodiments, less than 20% patients treated with the combination therapy disclosed herein discontinue the therapy due to TRAE. In certain embodiments, less than 20% patients treated with the combination therapy including a Trop-2 ADC and a Nectin-4 ADC discontinue the therapy due to TRAE. In certain embodiments, less than 30% patients treated with the combination therapy disclosed herein discontinue the therapy due to TRAE. In certain embodiments, less than 40% patients treated with the combination therapy disclosed herein discontinue the therapy due to TRAE. In certain embodiments, less than 50% patients treated with the combination therapy disclosed herein discontinue the therapy due to TRAE. In certain embodiments, the combination therapy disclosed herein is used to treat a patient, the patient does not discontinue the therapy due to TRAE. In certain embodiments, the combination therapy including a Trop-2 ADC and a Nectin-4 ADC is used to treat a patient, the patient does not discontinue the therapy due to TRAE. In certain embodiments, the combination therapy including SG and EV is used to treat a patient, the patient does not discontinue the therapy due to TRAE.

In certain embodiments, less than 20% patients treated with the combination therapy including SG and EV discontinue the therapy due to TRAE. In certain embodiments, less than 30% patients treated with the combination therapy including SG and EV discontinue the therapy due to TRAE. In certain embodiments, less than 40% patients treated with the combination therapy including SG and EV discontinue the therapy due to TRAE. In certain embodiments, less than 50% patients treated with the combination therapy including SG and EV discontinue the therapy due to TRAE.

In certain embodiment, the combination therapy including SG and EV disclosed herein does not have additional safety signals compared to using SG or EV alone. In certain embodiment, the combination therapy including SG and EV disclosed herein does not have additional TRAEs compared to using SG or EV alone.

In some embodiments, the subject or the patient is a human subject. In some embodiments, the subject or the patient is a laboratory animal.

### Efficacy

In certain embodiments, the present disclosure provides a method for treating relapsed or refractory la/mUC in a subject, including administering a combination therapy which includes Trop-2 ADC and Nectin-4 ADC, wherein the method achieves an improvement in clinical efficacy compared to the administration of the Trop-2 ADC or the Nectin-4 ADC alone for the treatment of relapsed or refractory la/mUC.

In certain embodiments, the present disclosure provides a method for treating relapsed or refractory la/mUC in a subject, including administering a combination therapy which includes SG and EV, wherein the method achieves an improvement in clinical efficacy compared to the administration of SG monotherapy or EV monotherapy for the treatment of relapsed or refractory la/mUC.

In certain embodiments, the present disclosure provides a method for treating la/mUC in a patient wherein la/mUC has progressed on platinum and/or immunotherapy, including administering a combination therapy which includes a Trop-2 ADC and a Nectin-4 ADC, wherein the method achieves an improvement in clinical efficacy compared to the administration of the Trop-2 ADC monotherapy or the Nectin-4 ADC monotherapy for the treatment of la/mUC.

In certain embodiments, the present disclosure provides a method for treating relapsed or refractory la/mUC in a patient wherein la/mUC has progressed on platinum and/or immunotherapy, including administering a combination therapy which includes SG and EV, wherein the method achieves an improvement in clinical efficacy compared to the administration of SG or EV alone for the treatment of la/mUC.

In certain embodiments, treatment of relapsed or refractory locally advanced or metastatic urothelial carcinoma (la/mUC) in a population of patients each having relapsed or refractory la/mUC with the combination therapy disclosed herein achieves an ORR of at least 60, 65, or 70%. In certain embodiments, treatment of relapsed or refractory la/mUC in a population of patients each having relapsed or refractory la/mUC with the combination therapy including a Trop-2 ADC and a Nectin-4 ADC achieves an ORR of at least 60, 65, or 70%. In certain embodiments, treatment of relapsed or refractory la/mUC in a population of patients each having relapsed or refractory la/mUC with the combination therapy including SG and EV achieves an ORR of at least 60, 65, or 70%. In certain embodiments, treatment of relapsed or refractory la/mUC in a population of patients each having relapsed or refractory la/mUC with the combination therapy including SG and EV achieves an ORR of at least 60%. In certain embodiments, treatment of relapsed or refractory la/mUC in a population of patients each having relapsed or refractory la/mUC with the combination therapy including SG and EV achieves an ORR of at least 65%. In certain embodiments, treatment of relapsed or refractory la/mUC in a population of patients each having relapsed or refractory la/mUC with the combination therapy including SG and EV achieves an ORR of at least 70%.

In certain embodiments, treatment of relapsed or refractory la/mUC in a population of patients each having relapsed or refractory la/mUC with the combination therapy disclosed herein achieves an ORR of 60-85%. In certain embodiments, treatment of relapsed or refractory la/mUC in a population of patients each having relapsed or refractory la/mUC with the combination therapy disclosed herein achieves an ORR of 70-85%. In certain embodiments, treatment of relapsed or refractory la/mUC in a population of patients each having relapsed or refractory la/mUC with the combination therapy disclosed herein achieves an ORR of 70-80%. In certain embodiments, treatment of relapsed or refractory la/mUC in a population of patients each having relapsed or refractory la/mUC with the combination therapy disclosed herein achieves an ORR of 70-75%. In certain embodiments, treatment of relapsed or refractory la/mUC in a population of patients each having relapsed or refractory la/mUC with the combination therapy disclosed herein achieves an ORR of 65-75%.

In certain embodiments, treatment of relapsed or refractory la/mUC in a population of patients each having relapsed or refractory la/mUC with the combination therapy including SG and EV achieves an ORR of 60-85%. In certain embodiments, treatment of relapsed or refractory la/mUC in a population of patients each having relapsed or refractory la/mUC with the combination therapy including SG and EV achieves an ORR of 70-85%. In certain embodiments, treatment of relapsed or refractory la/mUC in a population of patients each having relapsed or refractory la/mUC with the combination therapy including SG and EV achieves an ORR of 70-80%. In certain embodiments, treatment of relapsed or refractory la/mUC in a population of patients each having relapsed or refractory la/mUC with the combination therapy including SG and EV achieves an ORR of 70-75%. In certain embodiments, treatment of relapsed or refractory la/mUC in a population of patients each having relapsed or refractory la/mUC with the combination therapy including SG and EV achieves an ORR of 65-75%.

In certain embodiments, treatment of relapsed or refractory la/mUC in a population of patients each having relapsed or refractory la/mUC with the combination therapy disclosed herein achieves an ORR of at least 60% at a median 14-month follow-up.

In certain embodiments, treatment of relapsed or refractory la/mUC in a population of patients each having relapsed or refractory la/mUC with the combination therapy disclosed herein achieves a median DOR of greater than 12 months. In certain embodiments, treatment of relapsed or refractory la/mUC in a population of patients each having relapsed or refractory la/mUC with the combination therapy disclosed herein achieves a median DOR of greater than 14 months.

In certain embodiments, a patient having relapsed or refractory la/mUC treated with the combination therapy disclosed herein achieves a complete response for at least approximately 6 months. In certain embodiments, a patient having relapsed or refractory la/mUC treated with the combination therapy including SG and EV achieves a complete response for at least approximately 6 months.

In certain embodiments, when the combination therapy including SG and EV is used to treat la/mUC in a population of patients wherein la/mUC has progressed on platinum and/or immunotherapy, an ORR of at least 60, 65, or 70% is achieved. In certain embodiments, when the combination therapy including SG and EV is used to treat la/mUC in a population of patients wherein la/mUC has progressed on platinum and/or immunotherapy, an ORR of at least 60% is achieved. In certain embodiments, when the combination therapy including SG and EV is used to treat la/mUC in a population of patients wherein la/mUC has progressed on platinum and/or immunotherapy, an ORR of at least 65% is achieved. In certain embodiments, when the combination therapy including SG and EV is used to treat la/mUC in a population of patients wherein la/mUC has progressed on platinum and/or immunotherapy, an ORR of at least 70% is achieved.

In certain embodiments, when the combination therapy including SG and EV is used to treat la/mUC in a population of patients wherein la/mUC has progressed on platinum and/or immunotherapy, an ORR of 60-85% is achieved. In certain embodiments, when the combination therapy including SG and EV is used to treat la/mUC in a population of patients wherein la/mUC has progressed on platinum and/or immunotherapy, an ORR of 70-85% is achieved. In certain embodiments, when the combination therapy including SG and EV is used to treat la/mUC in a population of patients wherein la/mUC has progressed on platinum and/or immunotherapy, an ORR of 70-80% is achieved. In certain embodiments, when the combination therapy including SG and EV is used to treat la/mUC in a population of patients wherein la/mUC has progressed on platinum and/or immunotherapy, an ORR of 70-75% is achieved. In certain embodiments, when the combination therapy including SG and EV is used to treat la/mUC in a population of patients wherein la/mUC has progressed on platinum and/or immunotherapy, an ORR of 65-75% is achieved.

In certain embodiments, when the combination therapy including SG and EV is used to treat la/mUC in a population of patients wherein la/mUC has progressed on platinum and/or immunotherapy, an ORR of at least 60% is achieved at a median 14-month follow-up.

In certain embodiments, when the combination therapy including SG and EV is used to treat la/mUC in a population of patients wherein la/mUC has progressed on platinum and/or immunotherapy, a median DOR of greater than 12 months is achieved. In certain embodiments, when the combination therapy including SG and EV is used to treat la/mUC in a population of patients wherein la/mUC has progressed on platinum and/or immunotherapy, a median DOR of greater than 14 months is achieved. In certain embodiments, when the combination therapy including SG and EV is used to treat la/mUC in a patient wherein la/mUC has progressed on platinum and/or immunotherapy, a DOR of greater than 12 months is achieved. In certain embodiments, when the combination therapy including SG and EV is used to treat la/mUC in a patient wherein la/mUC has progressed on platinum and/or immunotherapy, a DOR of greater than 14 months is achieved.

In certain embodiments, when the combination therapy disclosed herein is used to treat la/mUC in a patient wherein la/mUC has progressed on platinum and/or immunotherapy, a complete response for at least approximately 6 months is achieved. In certain embodiments, when the combination therapy including SG and EV is used to treat la/mUC in a patient wherein la/mUC has progressed on platinum and/or immunotherapy, a complete response for at least approximately 6 months is achieved.

In some embodiments, the subject or the patient is a human subject. In some embodiments, the subject or the patient is a laboratory animal.

### SELECTED EXEMPLARY EMBODIMENTS

In one aspect, provided herein is a method of treating relapsed or refractory locally advanced or metastatic urothelial carcinoma in a human subject, comprising administering to the subject a combination therapy which comprises: (i) a therapeutically effective amount of a Trop-2 ADC and (ii) a therapeutically effective amount of a Nectin-4 ADC; wherein the Trop-2 ADC comprises a topoisomerase I inhibitor and the Nectin-4 ADC comprises a microtubule inhibitor; wherein the combination therapy does not comprise an anti-PD1 antibody or an anti-PD-L1 antibody, and wherein the subject has received one or more prior therapies for locally advanced or metastatic urothelial carcinoma. In some embodiments, the topoisomerase I inhibitor is SN38, exatecan, deruxtecan, or belotecan. In some embodiments, the topoisomerase I inhibitor is SN38. In some embodiments, the topoisomerase I inhibitor is exatecan. In some embodiments, the topoisomerase inhibitor is deruxtecan. In some embodiments, the topoisomerase I inhibitor is belotecan. In some embodiments, the Trop-2 ADC comprises sacituzumab or datopotamab. In some embodiments, the Trop-2 ADC comprises sacituzumab. In some embodiments, the Trop-2 ADC comprises datopotamab. In some embodiments, the Trop-2 ADC is sacituzumab govitecan (SG), sacituzumab tirumotecan (SKB264; sac-TMT), datopotamab deruxtecan (Dato-dxd), SHR-A1921, or ESG-401. In some embodiments, the Trop-2 ADC is sacituzumab govitecan (SG). In some embodiments, the Trop-2 ADC is sacituzumab tirumotecan (SKB264; sac-TMT). In some embodiments, the Trop-2 ADC is datopotamab deructecan (Dato-dxd). In some embodiments, the microtubule inhibitor is a maytansinoid or auristatin. In some embodiments, the maytansinoid is maytansine, mertansine/emtansine (DM1) or raytansine/soravtansine (DM4). In some embodiments, the auristatin is monomethyl auristatin E (MMAE) or monomethyl auristatin F (MMAF). In some embodiments, the microtubule inhibitor is monomethyl auristatin E (MMAE). In some embodiments, the Nectin-4 ADC is enfortumab vedotin (EV) (Seagen/Pfizer), CRB-701 (Corbus Pharmaceuticals), 9MW2821 (Mabwell), ADRX-0706 (Adcentrx), BT-8009 (BicycleTX), SYS-6002 (CSPC Megalith Biopharmaceutical), or ARC-101 (ARS Pharmaceuticals). In some embodiment, the Nectin-4 ADC comprises enfortumab. In some embodiments, the Nectin-4 ADC is enfortumab vedotin (EV). In some embodiments, the Trop-2 ADC is SG and the Nectin-4 ADC is EV. In some embodiments, the Trop-2 ADC is sac-TMT and the Nectin-4 ADC is EV. In some embodiments, the Trop-2 ADC is Dato-dxd and the Nectin-4 ADC is EV. In some embodiments, the metastatic or locally advanced urothelial carcinoma has progressed during or after each of the one or more prior therapies. In some embodiments, the subject has received prior platinum-based therapy for metastatic urothelial carcinoma, and the metastatic urothelial carcinoma has progressed during or after the platinum-based therapy. In some embodiments, the subject has received prior immunotherapy for metastatic urothelial carcinoma, and the metastatic urothelial carcinoma has progressed during or after the immunotherapy. In some embodiments, the locally advanced or metastatic urothelial carcinoma has relapsed after, or is refractory to, platinum-based chemotherapy and/or an anti-PD-1 antibody or anti-PD-L1 antibody (anti-PD(L)1 antibody). In some embodiments the anti-PD-1 antibody or anti-PD-L1 antibody is pembrolizumab, nivolumab, cemiplimab, pidilizumab, spartalizumab, sasanlimab, tislelizumab, retifanlimab, balstilimab, toripalimab, cetrelimab, genolimzumab, prolgolimab, camrelizumab, dostarlimab, sintilimab, zimberelimab, atezolizumab, avelumab, durvalumab, cosibelimab, lodapolimab, budigalimab, avelumab, or envafolimab. In some embodiments, the Trop-2 ADC and the Nectin-4 ADC are administered on day 1 and day 8 of a 21-day treatment cycle. In some embodiments, the combination therapy is administered until disease progression or unacceptable toxicity. In some embodiments, the combination therapy is administered for 10 or more, 11 or more, 12 or more, 13 or more, 14 or more, 15 or more, 16 or more, 17 or more, 18 or more, 19 or more, or 20 or more 21-day treatment cycles. In some embodiments, when the method is used to treat a population of subjects having locally advanced or metastatic urothelial carcinoma, the method achieves an overall response rate (ORR) of at least 60%, at least 65%, or at least 70%.

In one aspect, provided herein is a Trop-2 ADC for use in a method of treating relapsed or refractory locally advanced or metastatic urothelial carcinoma in a human subject, said method comprising administering to the subject: (i) the Trop-2 ADC and (ii) a Nectin-4 ADC; wherein the Trop-2 ADC comprises a topoisomerase I inhibitor and the Nectin-4 ADC comprises a microtubule inhibitor; wherein the method does not comprise administering to the subject an anti-PD1 antibody or an anti-PD-L1 antibody, and wherein the subject has received one or more prior therapies for locally advanced or metastatic urothelial carcinoma. In some embodiments, the topoisomerase I inhibitor is SN38, exatecan, deruxtecan, or belotecan. In some embodiments, the topoisomerase I inhibitor is SN38. In some embodiments, the topoisomerase I inhibitor is exatecan. In some embodiments, the topoisomerase inhibitor is deruxtecan. In some embodiments, the topoisomerase I inhibitor is belotecan. In some embodiments, the Trop-2 ADC comprises sacituzumab or datopotamab. In some embodiments, the Trop-2 ADC comprises sacituzumab. In some embodiments, the Trop-2 ADC comprises datopotamab. In some embodiments, the Trop-2 ADC is sacituzumab govitecan (SG), sacituzumab tirumotecan (SKB264; sac-TMT), datopotamab deruxtecan (Dato-dxd), SHR-A1921, or ESG-401. In some embodiments, the Trop-2 ADC is sacituzumab govitecan (SG). In some embodiments, the Trop-2 ADC is sacituzumab tirumotecan (SKB264; sac-TMT). In some embodiments, the Trop-2 ADC is datopotamab deructecan (Dato-dxd). In some embodiments, the microtubule inhibitor is a maytansinoid or auristatin. In some embodiments, the maytansinoid is maytansine, mertansine/emtansine (DM1) or raytansine/soravtansine (DM4). In some embodiments, the auristatin is monomethyl auristatin E (MMAE) or monomethyl auristatin F (MMAF). In some embodiments, the microtubule inhibitor is monomethyl auristatin E (MMAE). In some embodiments, the Nectin-4 ADC is enfortumab vedotin (EV) (Seagen/Pfizer), CRB-701 (Corbus Pharmaceuticals), 9MW2821 (Mabwell), ADRX-0706 (Adcentrx), BT-8009 (BicycleTX), SYS-6002 (CSPC Megalith Biopharmaceutical), or ARC-101 (ARS Pharmaceuticals). In some embodiment, the Nectin-4 ADC comprises enfortumab. In some embodiments, the Nectin-4 ADC is enfortumab vedotin (EV). In some embodiments, the Trop-2 ADC is SG and the Nectin-4 ADC is EV. In some embodiments, the Trop-2 ADC is sac-TMT and the Nectin-4 ADC is EV. In some embodiments, the Trop-2 ADC is Dato-dxd and the Nectin-4 ADC is EV. In some embodiments, the metastatic or locally advanced urothelial carcinoma has progressed during or after each of the one or more prior therapies. In some embodiments, the subject has received prior platinum-based therapy for metastatic urothelial carcinoma, and the metastatic urothelial carcinoma has progressed during or after the platinum-based therapy. In some embodiments, the subject has received prior immunotherapy for metastatic urothelial carcinoma, and the metastatic urothelial carcinoma has progressed during or after the immunotherapy. In some embodiments, the locally advanced or metastatic urothelial carcinoma has relapsed after, or is refractory to, platinum-based chemotherapy and/or an anti-PD-1 antibody or anti-PD-L1 antibody (anti-PD(L)1 antibody). In some embodiments the anti-PD-1 antibody or anti-PD-L1 antibody is pembrolizumab, nivolumab, cemiplimab, pidilizumab, spartalizumab, sasanlimab, tislelizumab, retifanlimab, balstilimab, toripalimab, cetrelimab, genolimzumab, prolgolimab, camrelizumab, dostarlimab, sintilimab, zimberelimab, atezolizumab, avelumab, durvalumab, cosibelimab, lodapolimab, budigalimab, avelumab, or envafolimab. In some embodiments, the Trop-2 ADC and the Nectin-4 ADC are administered on day 1 and day 8 of a 21-day treatment cycle. In some embodiments, the Trop-2 ADC and the Nectin-4 ADC are administered until disease progression or unacceptable toxicity. In some embodiments, the Trop-2 ADC and the Nectin-4 ADC are administered for 10 or more, 11 or more, 12 or more, 13 or more, 14 or more, 15 or more, 16 or more, 17 or more, 18 or more, 19 or more, or 20 or more 21-day treatment cycles. In some embodiments, when the Trop-2 ADC is used to treat a population of subjects having locally advanced or metastatic urothelial carcinoma, the method achieves an overall response rate (ORR) of at least 60%, at least 65%, or at least 70%.

In one aspect, provided herein is a Nectin-4 ADC for use in a method of treating relapsed or refractory locally advanced or metastatic urothelial carcinoma in a human subject, said method comprising administering to the subject: (i) a Trop-2 ADC and (ii) the Nectin-4 ADC; wherein the Trop-2 ADC comprises a topoisomerase I inhibitor and the Nectin-4 ADC comprises a microtubule inhibitor; wherein the method does not comprise administering to the subject an anti-PD1 antibody or an anti-PD-L1 antibody, and wherein the subject has received one or more prior therapies for locally advanced or metastatic urothelial carcinoma. In some embodiments, the topoisomerase I inhibitor is SN38, exatecan, deruxtecan, or belotecan. In some embodiments, the topoisomerase I inhibitor is SN38. In some embodiments, the topoisomerase I inhibitor is exatecan. In some embodiments, the topoisomerase inhibitor is deruxtecan. In some embodiments, the topoisomerase I inhibitor is belotecan. In some embodiments, the Trop-2 ADC comprises sacituzumab or datopotamab. In some embodiments, the Trop-2 ADC comprises sacituzumab. In some embodiments, the Trop-2 ADC comprises datopotamab. In some embodiments, the Trop-2 ADC is sacituzumab govitecan (SG), sacituzumab tirumotecan (SKB264; sac-TMT), datopotamab deruxtecan (Dato-dxd), SHR-A1921, or ESG-401. In some embodiments, the Trop-2 ADC is sacituzumab govitecan (SG). In some embodiments, the Trop-2 ADC is sacituzumab tirumotecan (SKB264; sac-TMT). In some embodiments, the Trop-2 ADC is datopotamab deructecan (Dato-dxd). In some embodiments, the microtubule inhibitor is a maytansinoid or auristatin. In some embodiments the maytansinoid is maytansine, mertansine/emtansine (DM1) or raytansine/soravtansine (DM4). In some embodiments the auristatin is monomethyl auristatin E (MMAE) or monomethyl auristatin F (MMAF). In some embodiments, the microtubule inhibitor is monomethyl auristatin E (MMAE). In some embodiments, the Nectin-4 ADC is enfortumab vedotin (EV) (Seagen/Pfizer), CRB-701 (Corbus Pharmaceuticals), 9MW2821 (Mabwell), ADRX-0706 (Adcentrx), BT-8009 (BicycleTX), SYS-6002 (CSPC Megalith Biopharmaceutical), or ARC-101 (ARS Pharmaceuticals). In some embodiments, the Nectin-4 ADC comprises enfortumab. In some embodiments, the Nectin-4 ADC is enfortumab vedotin (EV). In some embodiments, the Trop-2 ADC is SG and the Nectin-4 ADC is EV. In some embodiments, the Trop-2 ADC is sac-TMT and the Nectin-4 ADC is EV. In some embodiments, the Trop-2 ADC is Dato-dxd and the Nectin-4 ADC is EV. In some embodiments, the metastatic or locally advanced urothelial carcinoma has progressed during or after each of the one or more prior therapies. In some embodiments, the subject has received prior platinum-based therapy for metastatic urothelial carcinoma, and the metastatic urothelial carcinoma has progressed during or after the platinum-based therapy. In some embodiments, the subject has received prior immunotherapy for metastatic urothelial carcinoma, and the metastatic urothelial carcinoma has progressed during or after the immunotherapy. In some embodiments, the locally advanced or metastatic urothelial carcinoma has relapsed after, or is refractory to, platinum-based chemotherapy and/or an anti-PD-1 antibody or anti-PD-L1 antibody (anti-PD(L)1 antibody). In some embodiments the anti-PD-1 antibody or anti-PD-L1 antibody is pembrolizumab, nivolumab, cemiplimab, pidilizumab, spartalizumab, sasanlimab, tislelizumab, retifanlimab, balstilimab, toripalimab, cetrelimab, genolimzumab, prolgolimab, camrelizumab, dostarlimab, sintilimab, zimberelimab, atezolizumab, avelumab, durvalumab, cosibelimab, lodapolimab, budigalimab, avelumab, or envafolimab. In some embodiments, the Trop-2 ADC and the Nectin-4 ADC are administered on day 1 and day 8 of a 21-day treatment cycle. In some embodiments, the Trop-2 ADC and the Nectin-4 ADC are administered until disease progression or unacceptable toxicity. In some embodiments, the Trop-2 ADC and the Nectin-4 ADC are administered for 10 or more, 11 or more, 12 or more, 13 or more, 14 or more, 15 or more, 16 or more, 17 or more, 18 or more, 19 or more, or 20 or more 21-day treatment cycles. In some embodiments, when the Nectin-4 ADC is used to treat a population of subjects having locally advanced or metastatic urothelial carcinoma, the method achieves an overall response rate (ORR) of at least 60%, at least 65%, or at least 70%.

In one aspect, provided herein is a method of treating relapsed or refractory locally advanced or metastatic urothelial carcinoma in a human subject, comprising administering to the subject a combination therapy which comprises: (i) a therapeutically effective amount of a SG and (ii) a therapeutically effective amount of EV; wherein the combination therapy does not comprise an anti-PD1 antibody or an anti-PD-L1 antibody, and wherein the subject has received one or more prior therapies for locally advanced or metastatic urothelial carcinoma. In some embodiments, SG is administered at a dose of 5 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, the metastatic or locally advanced urothelial carcinoma has progressed during or after each of the one or more prior therapies. In some embodiments, the subject has received prior platinum-based therapy for metastatic urothelial carcinoma, and the metastatic urothelial carcinoma has progressed during or after the platinum-based therapy. In some embodiments, the subject has received prior immunotherapy for metastatic urothelial carcinoma, and the metastatic urothelial carcinoma has progressed during or after the immunotherapy. In some embodiments, the locally advanced or metastatic urothelial carcinoma has relapsed after, or is refractory to, platinum-based chemotherapy and/or an anti-PD-1 antibody or anti-PD-L1 antibody (anti-PD(L)1 antibody). In some embodiments the anti-PD-1 antibody or anti-PD-L1 antibody is pembrolizumab, nivolumab, cemiplimab, pidilizumab, spartalizumab, sasanlimab, tislelizumab, retifanlimab, balstilimab, toripalimab, cetrelimab, genolimzumab, prolgolimab, camrelizumab, dostarlimab, sintilimab, zimberelimab, atezolizumab, avelumab, durvalumab, cosibelimab, lodapolimab, budigalimab, avelumab, or envafolimab. In some embodiments, the combination therapy is administered until disease progression or unacceptable toxicity. In some embodiments, the combination therapy is administered for 10 or more, 11 or more, 12 or more, 13 or more, 14 or more, 15 or more, 16 or more, 17 or more, 18 or more, 19 or more, or 20 or more 21-day treatment cycles. In some embodiments, when the method is used to treat a population of subjects having locally advanced or metastatic urothelial carcinoma, the method achieves an overall respose rate (ORR) of at least 60%, at least 65%, or at least 70%.

In one aspect, provided herein is SG for use in a method of treating relapsed or refractory locally advanced or metastatic urothelial carcinoma in a human subject, said method comprising administering to the subject: (i) the SG and (ii) EV; wherein the method does not comprise an anti-PD1 antibody or an anti-PD-L1 antibody, and wherein the subject has received one or more prior therapies for locally advanced or metastatic urothelial carcinoma. In some embodiments, SG is administered at a dose of 5 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, the metastatic or locally advanced urothelial carcinoma has progressed during or after each of the one or more prior therapies. In some embodiments, the subject has received prior platinum-based therapy for metastatic urothelial carcinoma, and the metastatic urothelial carcinoma has progressed during or after the platinum-based therapy. In some embodiments, the subject has received prior immunotherapy for metastatic urothelial carcinoma, and the metastatic urothelial carcinoma has progressed during or after the immunotherapy. In some embodiments, the locally advanced or metastatic urothelial carcinoma has relapsed after, or is refractory to, platinum-based chemotherapy and/or an anti-PD-1 antibody or anti-PD-L1 antibody (anti-PD(L)1 antibody). In some embodiments the anti-PD-1 antibody or anti-PD-L1 antibody is pembrolizumab, nivolumab, cemiplimab, pidilizumab, spartalizumab, sasanlimab, tislelizumab, retifanlimab, balstilimab, toripalimab, cetrelimab, genolimzumab, prolgolimab, camrelizumab, dostarlimab, sintilimab, zimberelimab, atezolizumab, avelumab, durvalumab, cosibelimab, lodapolimab, budigalimab, avelumab, or envafolimab. In some embodiments, the Trop-2 ADC and the Nectin-4 ADC are administered until disease progression or unacceptable toxicity. In some embodiments, the Trop-2 ADC and the Nectin-4 ADC are administered for 10 or more, 11 or more, 12 or more, 13 or more, 14 or more, 15 or more, 16 or more, 17 or more, 18 or more, 19 or more, or 20 or more 21-day treatment cycles. In some embodiments, when the SG is used to treat a population of subjects having locally advanced or metastatic urothelial carcinoma, the method achieves an overall respose rate (ORR) of at least 60%, at least 65%, or at least 70%.

In one aspect, provided herein is EV for use in a method of treating relapsed or refractory locally advanced or metastatic urothelial carcinoma in a human subject, said method comprising administering to the subject: (i) SG and (ii) the EV; wherein the method does not comprise an anti-PD1 antibody or an anti-PD-L1 antibody, and wherein the subject has received one or more prior therapies for locally advanced or metastatic urothelial carcinoma. In some embodiments, SG is administered at a dose of 5 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, the metastatic or locally advanced urothelial carcinoma has progressed during or after each of the one or more prior therapies. In some embodiments, the subject has received prior platinum-based therapy for metastatic urothelial carcinoma, and the metastatic urothelial carcinoma has progressed during or after the platinum-based therapy. In some embodiments, the subject has received prior immunotherapy for metastatic urothelial carcinoma, and the metastatic urothelial carcinoma has progressed during or after the immunotherapy. In some embodiments, the locally advanced or metastatic urothelial carcinoma has relapsed after, or is refractory to, platinum-based chemotherapy and/or an anti-PD-1 antibody or anti-PD-L1 antibody (anti-PD(L)1 antibody). In some embodiments the anti-PD-1 antibody or anti-PD-L1 antibody is pembrolizumab, nivolumab, cemiplimab, pidilizumab, spartalizumab, sasanlimab, tislelizumab, retifanlimab, balstilimab, toripalimab, cetrelimab, genolimzumab, prolgolimab, camrelizumab, dostarlimab, sintilimab, zimberelimab, atezolizumab, avelumab, durvalumab, cosibelimab, lodapolimab, budigalimab, avelumab, or envafolimab. In some embodiments, the SG and EV are administered until disease progression or unacceptable toxicity. In some embodiments, the SG and EV are administered for 10 or more, 11 or more, 12 or more, 13 or more, 14 or more, 15 or more, 16 or more, 17 or more, 18 or more, 19 or more, or 20 or more 21-day treatment cycles. In some embodiments, when the EV is used to treat a population of subjects having locally advanced or metastatic urothelial carcinoma, the method achieves an overall respose rate (ORR) of at least 60%, at least 65%, or at least 70%.

In one aspect, provided herein is a method of treating locally advanced or metastatic urothelial carcinoma in a human subject, comprising administering to the subject a combination therapy which comprises: (i) a therapeutically effective amount of a Trop-2 ADC; (ii) a therapeutically effective amount of a Nectin-4 ADC, and (iii) a therapeutically effective amount of an anti-PD-1 antibody; wherein the Trop-2 ADC comprises a topoisomerase I inhibitor and the Nectin-4 ADC comprises a microtubule inhibitor; wherein the subject has not received a prior therapy for locally advanced or metastatic urothelial carcinoma. In some embodiments, the topoisomerase I inhibitor is SN38, exatecan, deruxtecan, or belotecan. In some embodiments, the topoisomerase I inhibitor is SN38. In some embodiments, the topoisomerase I inhibitor is exatecan. In some embodiments, the topoisomerase inhibitor is deruxtecan. In some embodiments, the topoisomerase I inhibitor is belotecan. In some embodiments, the Trop-2 ADC comprises sacituzumab or datopotamab. In some embodiments, the Trop-2 ADC comprises sacituzumab. In some embodiments, the Trop-2 ADC comprises datopotamab. In some embodiments, the Trop-2 ADC is sacituzumab govitecan (SG), sacituzumab tirumotecan (SKB264; sac-TMT), datopotamab deruxtecan (Dato-dxd), SHR-A1921, or ESG-401. In some embodiments, the Trop-2 ADC is sacituzumab govitecan (SG). In some embodiments, the Trop-2 ADC is sacituzumab tirumotecan (SKB264; sac-TMT). In some embodiments, the Trop-2 ADC is datopotamab deructecan (Dato-dxd). In some embodiments, the microtubule inhibitor is a maytansinoid or auristatin. In some embodiments the maytansinoid is maytansine, mertansine/emtansine (DM1) or raytansine/soravtansine (DM4). In some embodiments the auristatin is monomethyl auristatin E (MMAE) or monomethyl auristatin F (MMAF). In some embodiments, the microtubule inhibitor is monomethyl auristatin E (MMAE). In some embodiment, the Nectin-4 ADC comprises enfortumab. In some embodiments, the Nectin-4 ADC is enfortumab vedotin (EV) (Seagen/Pfizer), CRB-701 (Corbus Pharmaceuticals), 9MW2821 (Mabwell), ADRX-0706 (Adcentrx), BT-8009 (BicycleTX), SYS-6002 (CSPC Megalith Biopharmaceutical), or ARC-101 (ARS Pharmaceuticals). In some embodiments, the Nectin-4 ADC is enfortumab vedotin (EV). In some embodiments, the anti-PD-1 antibody is pembrolizumab. In some embodiments, the anti-PD-1 antibody is zimberelimab. In some embodiments, the Trop-2 ADC is SG, the Nectin-4 ADC is EV, and the anti-PD-1 antibody is pembrolizumab. In some embodiments, the Nectin-4 ADC is enfortumab vedotin (EV). In some embodiments, the Trop-2 ADC is SG; the Nectin-4 ADC is EV, and the anti-PD-1 antibody is zimberelimab. In some embodiments, the Trop-2 ADC is sac-TMT, the Nectin-4 ADC is EV, and the anti-PD-1 antibody is pembrolizumab. In some embodiments, the Trop-2 ADC is sac-TMT, the Nectin-4 ADC is EV, and the anti-PD-1 antibody is zimberelimab. In some embodiments, the Trop-2 ADC is Dato-dxd, the Nectin-4 ADC is EV, and the anti-PD-1 antibody is pembrolizumab. In some embodiments, the Trop-2 ADC is Dato-dxd, the Nectin-4 ADC is EV, and the anti-PD-1 antibody is zimberelimab. In some embodiments, the subject has received a prior therapy for urothelial carcinoma in a curative setting. In some embodiments, the subject is ineligible for or refusing cisplatin based therapy. In some embodiments, the combination therapy is administered for 10 or more, 11 or more, 12 or more, 13 or more, 14 or more, 15 or more, 16 or more, 17 or more, 18 or more, 19 or more, or 20 or more 21-day treatment cycles. In some embodiments, when the combination therapy is used to treat a population of subjects having locally advanced or metastatic urothelial carcinoma, the method achieves an overall response rate (ORR) of at least 60%, at least 65%, or at least 70%.

In one aspect, provided herein is a Trop-2 ADC for use in a method of treating relapsed or refractory locally advanced or metastatic urothelial carcinoma in a human subject, said method comprising administering to the subject: (i) the Trop-2 ADC; (ii) a Nectin-4 ADC, and (iii) an anti-PD-1 antibody; wherein the Trop-2 ADC comprises a topoisomerase I inhibitor and the Nectin-4 ADC comprises a microtubule inhibitor; and wherein the subject has not received a prior therapy for locally advanced or metastatic urothelial carcinoma. In some embodiments, the topoisomerase I inhibitor is SN38, exatecan, deruxtecan, or belotecan. In some embodiments, the topoisomerase I inhibitor is SN38. In some embodiments, the topoisomerase I inhibitor is exatecan. In some embodiments, the topoisomerase inhibitor is deruxtecan. In some embodiments, the topoisomerase I inhibitor is belotecan. In some embodiments, the Trop-2 ADC comprises sacituzumab or datopotamab. In some embodiments, the Trop-2 ADC comprises sacituzumab. In some embodiments, the Trop-2 ADC comprises datopotamab. In some embodiments, the Trop-2 ADC is sacituzumab govitecan (SG), sacituzumab tirumotecan (SKB264; sac-TMT), datopotamab deruxtecan (Dato-dxd), SHR-A1921, or ESG-401. In some embodiments, the Trop-2 ADC is sacituzumab govitecan (SG). In some embodiments, the Trop-2 ADC is sacituzumab tirumotecan (SKB264; sac-TMT). In some embodiments, the Trop-2 ADC is datopotamab deructecan (Dato-dxd). In some embodiments, the microtubule inhibitor is a maytansinoid or auristatin. In some embodiments the maytansinoid is maytansine, mertansine/emtansine (DM1) or raytansine/soravtansine (DM4). In some embodiments the auristatin is monomethyl auristatin E (MMAE) or monomethyl auristatin F (MMAF). In some embodiments, the microtubule inhibitor is monomethyl auristatin E (MMAE). In some embodiments, the microtubule inhibitor is monomethyl auristatin E (MMAE). In some embodiment, the Nectin-4 ADC comprises enfortumab. In some embodiments, the Nectin-4 ADC is enfortumab vedotin (EV) (Seagen/Pfizer), CRB-701 (Corbus Pharmaceuticals), 9MW2821 (Mabwell), ADRX-0706 (Adcentrx), BT-8009 (BicycleTX), SYS-6002 (CSPC Megalith Biopharmaceutical), or ARC-101 (ARS Pharmaceuticals). In some embodiments, the Nectin-4 ADC is enfortumab vedotin (EV). In some embodiments, the anti-PD-1 antibody is pembrolizumab. In some embodiments, the anti-PD-1 antibody is zimberelimab. In some embodiments, the Trop-2 ADC is SG, the Nectin-4 ADC is EV, and the anti-PD-1 antibody is pembrolizumab. In some embodiments, the Nectin-4 ADC is enfortumab vedotin (EV). In some embodiments, the Trop-2 ADC is SG; the Nectin-4 ADC is EV, and the anti-PD-1 antibody is zimberelimab. In some embodiments, the Trop-2 ADC is sac-TMT, the Nectin-4 ADC is EV, and the anti-PD-1 antibody is pembrolizumab. In some embodiments, the Trop-2 ADC is sac-TMT, the Nectin-4 ADC is EV, and the anti-PD-1 antibody is zimberelimab. In some embodiments, the Trop-2 ADC is Dato-dxd, the Nectin-4 ADC is EV, and the anti-PD-1 antibody is pembrolizumab. In some embodiments, the Trop-2 ADC is Dato-dxd, the Nectin-4 ADC is EV, and the anti-PD-1 antibody is zimberelimab. In some embodiments, the subject has received a prior therapy for urothelial carcinoma in a curative setting. In some embodiments, the subject is ineligible for or refusing cisplatin based therapy. In some embodiments, the Trop-2 ADC, Nectin-4 ADC, and anti-PD-1 antibody and are administered for 10 or more, 11 or more, 12 or more, 13 or more, 14 or more, 15 or more, 16 or more, 17 or more, 18 or more, 19 or more, or 20 or more 21-day treatment cycles. In some embodiments, when the Trop-2 ADC, Nectin-4 ADC, and anti-PD-1 antibody are used to treat a population of subjects having locally advanced or metastatic urothelial carcinoma, the method achieves an overall response rate (ORR) of at least 60%, at least 65%, or at least 70%.

In one aspect, provided herein is a Nectin-4 ADC for use in a method of treating relapsed or refractory locally advanced or metastatic urothelial carcinoma in a human subject, said method comprising administering to the subject: (i) a Trop-2 ADC; (ii) the Nectin-4 ADC, and (iii) an anti-PD-1 antibody; wherein the Trop-2 ADC comprises a topoisomerase I inhibitor and the Nectin-4 ADC comprises a microtubule inhibitor; and wherein the subject has not received a prior therapy for locally advanced or metastatic urothelial carcinoma. In some embodiments, the topoisomerase I inhibitor is SN38, exatecan, deruxtecan, or belotecan. In some embodiments, the topoisomerase I inhibitor is SN38. In some embodiments, the topoisomerase I inhibitor is exatecan. In some embodiments, the topoisomerase inhibitor is deruxtecan. In some embodiments, the topoisomerase I inhibitor is belotecan. In some embodiments, the Trop-2 ADC comprises sacituzumab or datopotamab. In some embodiments, the Trop-2 ADC comprises sacituzumab. In some embodiments, the Trop-2 ADC comprises datopotamab. In some embodiments, the Trop-2 ADC is sacituzumab govitecan (SG), sacituzumab tirumotecan (SKB264; sac-TMT), datopotamab deruxtecan (Dato-dxd), SHR-A1921, or ESG-401. In some embodiments, the Trop-2 ADC is sacituzumab govitecan (SG). In some embodiments, the Trop-2 ADC is sacituzumab tirumotecan (SKB264; sac-TMT). In some embodiments, the Trop-2 ADC is datopotamab deructecan (Dato-dxd). In some embodiments, the microtubule inhibitor is a maytansinoid or auristatin. In some embodiments the maytansinoid is maytansine, mertansine/emtansine (DM1) or raytansine/soravtansine (DM4). In some embodiments the auristatin is monomethyl auristatin E (MMAE) or monomethyl auristatin F (MMAF). In some embodiments, the microtubule inhibitor is monomethyl auristatin E (MMAE). In some embodiment, the Nectin-4 ADC comprises enfortumab. In some embodiments, the Nectin-4 ADC is enfortumab vedotin (EV) (Seagen/Pfizer), CRB-701 (Corbus Pharmaceuticals), 9MW2821 (Mabwell), ADRX-0706 (Adcentrx), BT-8009 (BicycleTX), SYS-6002 (CSPC Megalith Biopharmaceutical), or ARC-101 (ARS Pharmaceuticals). In some embodiments, the Nectin-4 ADC is enfortumab vedotin (EV). In some embodiments, the anti-PD-1 antibody is pembrolizumab. In some embodiments, the anti-PD-1 antibody is zimberelimab. In some embodiments, the Trop-2 ADC is SG, the Nectin-4 ADC is EV, and the anti-PD-1 antibody is pembrolizumab. In some embodiments, the Nectin-4 ADC is enfortumab vedotin (EV). In some embodiments, the Trop-2 ADC is SG; the Nectin-4 ADC is EV, and the anti-PD-1 antibody is zimberelimab. In some embodiments, the Trop-2 ADC is sac-TMT, the Nectin-4 ADC is EV, and the anti-PD-1 antibody is pembrolizumab. In some embodiments, the Trop-2 ADC is sac-TMT, the Nectin-4 ADC is EV, and the anti-PD-1 antibody is zimberelimab. In some embodiments, the Trop-2 ADC is Dato-dxd, the Nectin-4 ADC is EV, and the anti-PD-1 antibody is pembrolizumab. In some embodiments, the Trop-2 ADC is Dato-dxd, the Nectin-4 ADC is EV, and the anti-PD-1 antibody is zimberelimab. In some embodiments, the subject has received a prior therapy for urothelial carcinoma in a curative setting. In some embodiments, the subject is ineligible for or refusing cisplatin based therapy. In some embodiments, the Trop-2 ADC, Nectin-4 ADC, and anti-PD-1 antibody and are administered for 10 or more, 11 or more, 12 or more, 13 or more, 14 or more, 15 or more, 16 or more, 17 or more, 18 or more, 19 or more, or 20 or more 21-day treatment cycles. In some embodiments, when the Trop-2 ADC, Nectin-4 ADC, and anti-PD-1 antibody are used to treat a population of subjects having locally advanced or metastatic urothelial carcinoma, the method achieves an overall response rate (ORR) of at least 60%, at least 65%, or at least 70%.

In one aspect, provided herein is an anti-PD-1 antibody for use in a method of treating relapsed or refractory locally advanced or metastatic urothelial carcinoma in a human subject, said method comprising administering to the subject: (i) a Trop-2 ADC; (ii) a Nectin-4 ADC, and (iii) the anti-PD-1 antibody; wherein the Trop-2 ADC comprises a topoisomerase I inhibitor and the Nectin-4 ADC comprises a microtubule inhibitor; and wherein the subject has not received a prior therapy for locally advanced or metastatic urothelial carcinoma. In some embodiments, the topoisomerase I inhibitor is SN38, exatecan, deruxtecan, or belotecan. In some embodiments, the topoisomerase I inhibitor is SN38. In some embodiments, the topoisomerase I inhibitor is exatecan. In some embodiments, the topoisomerase inhibitor is deruxtecan. In some embodiments, the topoisomerase I inhibitor is belotecan. In some embodiments, the Trop-2 ADC comprises sacituzumab or datopotamab. In some embodiments, the Trop-2 ADC comprises sacituzumab. In some embodiments, the Trop-2 ADC comprises datopotamab. In some embodiments, the Trop-2 ADC is sacituzumab govitecan (SG), sacituzumab tirumotecan (SKB264; sac-TMT), datopotamab deruxtecan (Dato-dxd), SHR-A1921, or ESG-401. In some embodiments, the Trop-2 ADC is sacituzumab govitecan (SG). In some embodiments, the Trop-2 ADC is sacituzumab tirumotecan (SKB264; sac-TMT). In some embodiments, the Trop-2 ADC is datopotamab deructecan (Dato-dxd). In some embodiments, the microtubule inhibitor is a maytansinoid or auristatin. In some embodiments the maytansinoid is maytansine, mertansine/emtansine (DM1) or raytansine/soravtansine (DM4). In some embodiments the auristatin is monomethyl auristatin E (MMAE) or monomethyl auristatin F (MMAF). In some embodiments, the microtubule inhibitor is monomethyl auristatin E (MMAE). In some embodiment, the Nectin-4 ADC comprises enfortumab. In some embodiments, the Nectin-4 ADC is enfortumab vedotin (EV) (Seagen/Pfizer), CRB-701 (Corbus Pharmaceuticals), 9MW2821 (Mabwell), ADRX-0706 (Adcentrx), BT-8009 (BicycleTX), SYS-6002 (CSPC Megalith Biopharmaceutical), or ARC-101 (ARS Pharmaceuticals). In some embodiments, the Nectin-4 ADC is enfortumab vedotin (EV). In some embodiments, the anti-PD-1 antibody is pembrolizumab. In some embodiments, the anti-PD-1 antibody is zimberelimab. In some embodiments, the Trop-2 ADC is SG, the Nectin-4 ADC is EV, and the anti-PD-1 antibody is pembrolizumab. In some embodiments, the Nectin-4 ADC is enfortumab vedotin (EV). In some embodiments, the Trop-2 ADC is SG; the Nectin-4 ADC is EV, and the anti-PD-1 antibody is zimberelimab. In some embodiments, the Trop-2 ADC is sac-TMT, the Nectin-4 ADC is EV, and the anti-PD-1 antibody is pembrolizumab. In some embodiments, the Trop-2 ADC is sac-TMT, the Nectin-4 ADC is EV, and the anti-PD-1 antibody is zimberelimab. In some embodiments, the Trop-2 ADC is Dato-dxd, the Nectin-4 ADC is EV, and the anti-PD-1 antibody is pembrolizumab. In some embodiments, the Trop-2 ADC is Dato-dxd, the Nectin-4 ADC is EV, and the anti-PD-1 antibody is zimberelimab. In some embodiments, the subject has received a prior therapy for urothelial carcinoma in a curative setting. In some embodiments, the subject is ineligible for or refusing cisplatin based therapy. In some embodiments, the Trop-2 ADC, Nectin-4 ADC, and anti-PD-1 antibody and are administered for 10 or more, 11 or more, 12 or more, 13 or more, 14 or more, 15 or more, 16 or more, 17 or more, 18 or more, 19 or more, or 20 or more 21-day treatment cycles. In some embodiments, when the Trop-2 ADC, Nectin-4 ADC, and anti-PD-1 antibody are used to treat a population of subjects having locally advanced or metastatic urothelial carcinoma, the method achieves an overall response rate (ORR) of at least 60%, at least 65%, or at least 70%.

In one aspect, provided herein is a method of treating locally advanced or metastatic urothelial carcinoma in a human subject, comprising administering to the subject a combination therapy which comprises: (i) a therapeutically effective amount of SG; (ii) a therapeutically effective amount of EV, and (iii) a therapeutically effective amount of an anti-PD-1 antibody; wherein the subject has not received a prior therapy for locally advanced or metastatic urothelial carcinoma. In some embodiments, SG is administered at a dose of 5 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, the anti-PD-1 antibody is pembrolizumab. In some embodiments, pembrolizumab is administered at a dose level of about 200 mg per dose as an intravenous infusion on day 1 of the 21-day treatment cycle. In some embodiments, zimberelimab is administered at a dose level of about 360 mg per dose as an intravenous infusion on day 1 of the 21-day treatment cycle. In some embodiments, SG is administered at a dose of 5 mg/kg as an intravenous infusion on day 1 and day 8 of a 21-day treatment cycle; EV is administered at a dose of 1.25 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 5 mg/kg as an intravenous infusion on day 1 and day 8 of a 21-day treatment cycle; EV is administered at a dose of 1 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 5 mg/kg as an intravenous infusion on day 1 and day 8 of a 21-day treatment cycle; EV is administered at a dose of 0.75 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 5 mg/kg as an intravenous infusion on day 1 and day 8 of a 21-day treatment cycle; EV is administered at a dose of 0.5 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 7.5 mg/kg as an intravenous infusion on day 1 and day 8 of a 21-day treatment cycle; EV is administered at a dose of 1.25 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 7.5 mg/kg as an intravenous infusion on day 1 and day 8 of a 21-day treatment cycle; EV is administered at a dose of 1 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 7.5 mg/kg as an intravenous infusion on day 1 and day 8 of a 21-day treatment cycle; EV is administered at a dose of 0.75 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 7.5 mg/kg as an intravenous infusion on day 1 and day 8 of a 21-day treatment cycle; EV is administered at a dose of 0.5 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 8 mg/kg as an intravenous infusion on day 1 and day 8 of a 21-day treatment cycle; EV is administered at a dose of 1.25 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 8 mg/kg as an intravenous infusion on day 1 and day 8 of a 21-day treatment cycle; EV is administered at a dose of 1 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 8 mg/kg as an intravenous infusion on day 1 and day 8 of a 21-day treatment cycle; EV is administered at a dose of 0.75 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 8 mg/kg as an intravenous infusion on day 1 and day 8 of a 21-day treatment cycle; EV is administered at a dose of 0.5 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 10 mg/kg as an intravenous infusion on day 1 and day 8 of a 21-day treatment cycle; EV is administered at a dose of 1.25 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 10 mg/kg as an intravenous infusion on day 1 and day 8 of a 21-day treatment cycle; EV is administered at a dose of 1 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 10 mg/kg as an intravenous infusion on day 1 and day 8 of a 21-day treatment cycle; EV is administered at a dose of 0.75 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 10 mg/kg as an intravenous infusion on day 1 and day 8 of a 21-day treatment cycle; EV is administered at a dose of 0.5 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, the subject has received a prior therapy for urothelial carcinoma in a curative setting. In some embodiments, the subject is ineligible for or refusing cisplatin based therapy. In some embodiments, the combination therapy is administered for 10 or more, 11 or more, 12 or more, 13 or more, 14 or more, 15 or more, 16 or more, 17 or more, 18 or more, 19 or more, or 20 or more 21-day treatment cycles. In some embodiments, when the combination therapy is used to treat a population of subjects having locally advanced or metastatic urothelial carcinoma, the method achieves an overall response rate (ORR) of at least 60%, at least 65%, or at least 70%.

In one aspect, provided herein is a SG for use in a method of treating locally advanced or metastatic urothelial carcinoma in a human subject, said method comprising administering to the subject (i) the SG; (ii) EV, and (iii) an anti-PD-1 antibody; wherein the subject has not received a prior therapy for locally advanced or metastatic urothelial carcinoma. In some embodiments, SG is administered at a dose of 5 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, the anti-PD-1 antibody is pembrolizumab. In some embodiments, pembrolizumab is administered at a dose level of about 200 mg per dose as an intravenous infusion on day 1 of the 21-day treatment cycle. In some embodiments, zimberelimab is administered at a dose level of about 360 mg per dose as an intravenous infusion on day 1 of the 21-day treatment cycle. In some embodiments, SG is administered at a dose of 5 mg/kg as an intravenous infusion on day 1 and day 8 of a 21-day treatment cycle; EV is administered at a dose of 1.25 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 5 mg/kg as an intravenous infusion on day 1 and day 8 of a 21-day treatment cycle; EV is administered at a dose of 1 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 5 mg/kg as an intravenous infusion on day 1 and day 8 of a 21-day treatment cycle; EV is administered at a dose of 0.75 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 5 mg/kg as an intravenous infusion on day 1 and day 8 of a 21-day treatment cycle; EV is administered at a dose of 0.5 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 7.5 mg/kg as an intravenous infusion on day 1 and day 8 of a 21-day treatment cycle; EV is administered at a dose of 1.25 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 7.5 mg/kg as an intravenous infusion on day 1 and day 8 of a 21-day treatment cycle; EV is administered at a dose of 1 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 7.5 mg/kg as an intravenous infusion on day 1 and day 8 of a 21-day treatment cycle; EV is administered at a dose of 0.75 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 7.5 mg/kg as an intravenous infusion on day 1 and day 8 of a 21-day treatment cycle; EV is administered at a dose of 0.5 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 8 mg/kg as an intravenous infusion on day 1 and day 8 of a 21-day treatment cycle; EV is administered at a dose of 1.25 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 8 mg/kg as an intravenous infusion on day 1 and day 8 of a 21-day treatment cycle; EV is administered at a dose of 1 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 8 mg/kg as an intravenous infusion on day 1 and day 8 of a 21-day treatment cycle; EV is administered at a dose of 0.75 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 8 mg/kg as an intravenous infusion on day 1 and day 8 of a 21-day treatment cycle; EV is administered at a dose of 0.5 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 10 mg/kg as an intravenous infusion on day 1 and day 8 of a 21-day treatment cycle; EV is administered at a dose of 1.25 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 10 mg/kg as an intravenous infusion on day 1 and day 8 of a 21-day treatment cycle; EV is administered at a dose of 1 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 10 mg/kg as an intravenous infusion on day 1 and day 8 of a 21-day treatment cycle; EV is administered at a dose of 0.75 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 10 mg/kg as an intravenous infusion on day 1 and day 8 of a 21-day treatment cycle; EV is administered at a dose of 0.5 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, the subject has received a prior therapy for urothelial carcinoma in a curative setting. In some embodiments, the subject is ineligible for or refusing cisplatin based therapy. In some embodiments, the SG, EV, and anti-PD-1 antibody are administered for 10 or more, 11 or more, 12 or more, 13 or more, 14 or more, 15 or more, 16 or more, 17 or more, 18 or more, 19 or more, or 20 or more 21-day treatment cycles. In some embodiments, when the SG, EV, anti-PD-1 antibody are used to treat a population of subjects having locally advanced or metastatic urothelial carcinoma, the method achieves an overall response rate (ORR) of at least 60%, at least 65%, or at least 70%.

In one aspect, provided herein is EV for use in a method of treating locally advanced or metastatic urothelial carcinoma in a human subject, said method comprising administering to the subject (i) SG; (ii) the EV, and (iii) an anti-PD-1 antibody; wherein the subject has not received a prior therapy for locally advanced or metastatic urothelial carcinoma. In some embodiments, SG is administered at a dose of 5 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, the anti-PD-1 antibody is pembrolizumab. In some embodiments, pembrolizumab is administered at a dose level of about 200 mg per dose as an intravenous infusion on day 1 of the 21-day treatment cycle. In some embodiments, zimberelimab is administered at a dose level of about 360 mg per dose as an intravenous infusion on day 1 of the 21-day treatment cycle. In some embodiments, SG is administered at a dose of 5 mg/kg as an intravenous infusion on day 1 and day 8 of a 21-day treatment cycle; EV is administered at a dose of 1.25 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 5 mg/kg as an intravenous infusion on day 1 and day 8 of a 21-day treatment cycle; EV is administered at a dose of 1 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 5 mg/kg as an intravenous infusion on day 1 and day 8 of a 21-day treatment cycle; EV is administered at a dose of 0.75 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 5 mg/kg as an intravenous infusion on day 1 and day 8 of a 21-day treatment cycle; EV is administered at a dose of 0.5 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 7.5 mg/kg as an intravenous infusion on day 1 and day 8 of a 21-day treatment cycle; EV is administered at a dose of 1.25 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 7.5 mg/kg as an intravenous infusion on day 1 and day 8 of a 21-day treatment cycle; EV is administered at a dose of 1 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 7.5 mg/kg as an intravenous infusion on day 1 and day 8 of a 21-day treatment cycle; EV is administered at a dose of 0.75 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 7.5 mg/kg as an intravenous infusion on day 1 and day 8 of a 21-day treatment cycle; EV is administered at a dose of 0.5 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 8 mg/kg as an intravenous infusion on day 1 and day 8 of a 21-day treatment cycle; EV is administered at a dose of 1.25 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 8 mg/kg as an intravenous infusion on day 1 and day 8 of a 21-day treatment cycle; EV is administered at a dose of 1 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 8 mg/kg as an intravenous infusion on day 1 and day 8 of a 21-day treatment cycle; EV is administered at a dose of 0.75 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 8 mg/kg as an intravenous infusion on day 1 and day 8 of a 21-day treatment cycle; EV is administered at a dose of 0.5 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 10 mg/kg as an intravenous infusion on day 1 and day 8 of a 21-day treatment cycle; EV is administered at a dose of 1.25 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 10 mg/kg as an intravenous infusion on day 1 and day 8 of a 21-day treatment cycle; EV is administered at a dose of 1 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 10 mg/kg as an intravenous infusion on day 1 and day 8 of a 21-day treatment cycle; EV is administered at a dose of 0.75 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 10 mg/kg as an intravenous infusion on day 1 and day 8 of a 21-day treatment cycle; EV is administered at a dose of 0.5 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, the subject has received a prior therapy for urothelial carcinoma in a curative setting. In some embodiments, the subject is ineligible for or refusing cisplatin based therapy. In some embodiments, the SG, EV, and anti-PD-1 antibody are administered for 10 or more, 11 or more, 12 or more, 13 or more, 14 or more, 15 or more, 16 or more, 17 or more, 18 or more, 19 or more, or 20 or more 21-day treatment cycles. In some embodiments, when the SG, EV, anti-PD-1 antibody are used to treat a population of subjects having locally advanced or metastatic urothelial carcinoma, the method achieves an overall response rate (ORR) of at least 60%, at least 65%, or at least 70%.

In one aspect, provided herein is an anti-PD-1 antibody for use in a method of treating locally advanced or metastatic urothelial carcinoma in a human subject, said method comprising administering to the subject (i) SG; (ii) EV, and (iii) the anti-PD-1 antibody; wherein the subject has not received a prior therapy for locally advanced or metastatic urothelial carcinoma. In some embodiments, SG is administered at a dose of 5 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 5 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 7.5 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 8 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 1.25 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 1 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 0.75 mg/kg as an intravenous infusion on day 1 and day 8 of the 21-day treatment cycle. In some embodiments, SG is administered at a dose of 10 mg/kg as an intravenous infusion on day 1 and day 8 of a 21-day treatment cycle; and EV is administered at a dose of 0.5 mg/kg as an intravenous infusion on day 1 and day 8 of the 21-day treatment cycle. In some embodiments, the anti-PD-1 antibody is pembrolizumab. In some embodiments, pembrolizumab is administered at a dose level of about 200 mg per dose as an intravenous infusion on day 1 of the 21-day treatment cycle. In some embodiments, zimberelimab is administered at a dose level of about 360 mg per dose as an intravenous infusion on day 1 of the 21-day treatment cycle. In some embodiments, SG is administered at a dose of 5 mg/kg as an intravenous infusion on day 1 and day 8 of a 21-day treatment cycle; EV is administered at a dose of 1.25 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 5 mg/kg as an intravenous infusion on day 1 and day 8 of a 21-day treatment cycle; EV is administered at a dose of 1 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 5 mg/kg as an intravenous infusion on day 1 and day 8 of a 21-day treatment cycle; EV is administered at a dose of 0.75 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 5 mg/kg as an intravenous infusion on day 1 and day 8 of a 21-day treatment cycle; EV is administered at a dose of 0.5 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 7.5 mg/kg as an intravenous infusion on day 1 and day 8 of a 21-day treatment cycle; EV is administered at a dose of 1.25 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 7.5 mg/kg as an intravenous infusion on day 1 and day 8 of a 21-day treatment cycle; EV is administered at a dose of 1 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 7.5 mg/kg as an intravenous infusion on day 1 and day 8 of a 21-day treatment cycle; EV is administered at a dose of 0.75 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 7.5 mg/kg as an intravenous infusion on day 1 and day 8 of a 21-day treatment cycle; EV is administered at a dose of 0.5 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 8 mg/kg as an intravenous infusion on day 1 and day 8 of a 21-day treatment cycle; EV is administered at a dose of 1.25 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 8 mg/kg as an intravenous infusion on day 1 and day 8 of a 21-day treatment cycle; EV is administered at a dose of 1 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 8 mg/kg as an intravenous infusion on day 1 and day 8 of a 21-day treatment cycle; EV is administered at a dose of 0.75 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 8 mg/kg as an intravenous infusion on day 1 and day 8 of a 21-day treatment cycle; EV is administered at a dose of 0.5 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 10 mg/kg as an intravenous infusion on day 1 and day 8 of a 21-day treatment cycle; EV is administered at a dose of 1.25 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 10 mg/kg as an intravenous infusion on day 1 and day 8 of a 21-day treatment cycle; EV is administered at a dose of 1 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 10 mg/kg as an intravenous infusion on day 1 and day 8 of a 21-day treatment cycle; EV is administered at a dose of 0.75 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, SG is administered at a dose of 10 mg/kg as an intravenous infusion on day 1 and day 8 of a 21-day treatment cycle; EV is administered at a dose of 0.5 mg/kg as an intravenous infusion on day 1 and day 8 of the 21-day treatment cycle, and pembrolizumab is administered at a dose level of about 200 mg per dose as an intravenous infusion on day 1 of the 21-day treatment cycle or zimberelimab is administered at a dose level of about 360 mg per dose as an intravenous infusion on day 1 of the 21-day treatment cycle. In some embodiments, the subject has received a prior therapy for urothelial carcinoma in a curative setting. In some embodiments, the subject is ineligible for or refusing cisplatin based therapy. In some embodiments, the SG, EV, and anti-PD-1 antibody are administered for 10 or more, 11 or more, 12 or more, 13 or more, 14 or more, 15 or more, 16 or more, 17 or more, 18 or more, 19 or more, or 20 or more 21-day treatment cycles. In some embodiments, when the SG, EV, anti-PD-1 antibody are used to treat a population of subjects having locally advanced or metastatic urothelial carcinoma, the method achieves an overall response rate (ORR) of at least 60%, at least 65%, or at least 70%.

### ADDITIONAL EXEMPLARY EMBODIMENTS

Embodiment 1: A method of treating relapsed or refractory locally advanced or metastatic urothelial carcinoma in a human subject, comprising administering to the subject a combination therapy which comprises: (i) a therapeutically effective amount of a Trop-2 antibody-drug conjugate (Trop-2 ADC); and (ii) a therapeutically effective amount of a Nectin-4 antibody-drug conjugate (Nectin-4 ADC).

Embodiment 2: A method of safely treating relapsed or refractory locally advanced or metastatic urothelial carcinoma in a human subject, comprising administering to the subject a combination therapy which comprises: (i) a therapeutically effective amount of a Trop-2 antibody-drug conjugate (Trop-2 ADC); and (ii) a therapeutically effective amount of a Nectin-4 antibody-drug conjugate (Nectin-4 ADC).

Embodiment 3. A method of treating relapsed or refractory locally advanced or metastatic urothelial carcinoma that is well tolerated in a human subject, comprising administering to the subject a combination therapy which comprises: (i) a therapeutically effective amount of a Trop-2 antibody-drug conjugate (Trop-2 ADC); and (ii) a therapeutically effective amount of a Nectin-4 antibody-drug conjugate (Nectin-4 ADC).

Embodiment 4: A method of improving clinical efficacy of a Trop-2 ADC monotherapy or a Nectin-4 ADC monotherapy for treating relapsed or refractory locally advanced or metastatic urothelial carcinoma in a human subject, comprising administering to the subject a combination therapy which comprises: (i) a therapeutically effective amount of a Trop-2 antibody-drug conjugate (Trop-2 ADC); and (ii) a therapeutically effective amount of a Nectin-4 antibody-drug conjugate (Nectin-4 ADC).

Embodiment 5: A method of treating relapsed or refractory locally advanced or metastatic urothelial carcinoma in a human subject using a combination therapy without raising an additional safety signal compared to a Trop-2 ADC monotherapy or a Nectin-4 ADC monotherapy, comprising administering to the subject the combination therapy which comprises: (i) a therapeutically effective amount of a Trop-2 antibody-drug conjugate (Trop-2 ADC); and (ii) a therapeutically effective amount of a Nectin-4 antibody-drug conjugate (Nectin-4 ADC).

Embodiment 6: The method of any one of Embodiments 1 to 5, wherein the Trop-2 ADC comprises an anti Trop-2 antibody or an antigen-binding fragment thereof, wherein the anti Trop-2 antibody or the antigen binding fragment thereof comprises a light chain comprising SEQ ID NO: 13 and a heavy chain comprising SEQ ID NO:14.

Embodiment 7: The method of any one of Embodiments 1 to 6, wherein the anti-Trop-2 antibody is sacituzumab.

Embodiment 8: The method of any one of Embodiments 1 to 5, wherein the anti-Trop-2 antibody is datopotamab.

Embodiment 9: The method of any one of Embodiments 1 to 8, wherein the Trop-2 ADC comprises a topoisomerase I inhibitor.

Embodiment 10: The method of Embodiment 9, wherein the topoisomerase I inhibitor is SN38, deruxtecan, or belotecan.

Embodiment 11: The method of Embodiment 8, wherein the topoisomerase I inhibitor is SN38.

Embodiment 12: The method of any one of Embodiments 1 to 5, wherein the Trop-2 ADC is sacituzumab govitecan, sacituzumab tirumotecan (SKB264), or datopotamab deruxtecan.

Embodiment 13: The method of any one of Embodiments 1 to 5, wherein the Trop-2 ADC is sacituzumab govitecan (SG).

Embodiment 14: The method of any one of Embodiments 1 to 5, wherein the Trop-2 ADC comprises an anti Trop-2 antibody or an antigen-binding fragment thereof, wherein the anti Trop-2 antibody or the antigen binding fragment thereof comprises light chain complementarity determining regions (CDRs) comprising a sequence of amino acids as set forth in SEQ ID NOs: 1, 2 and 3 and heavy chain CDRs comprising a sequence of amino acids as set forth in SEQ ID NOs: 4, 5 and 6, and wherein the Trop-2 ADC has a formula represented by:

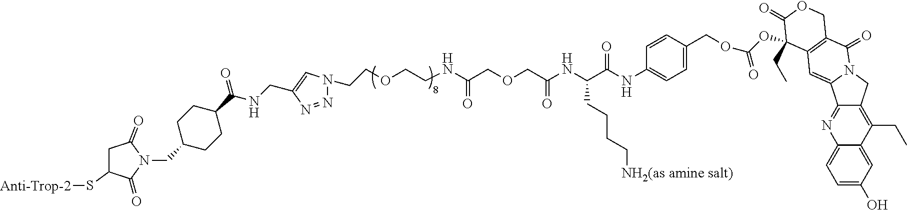

Embodiment 15: The method of any one of Embodiments 1 to 14, wherein the Nectin-4 ADC comprises an anti-Nectin-4 antibody or an antigen-binding fragment thereof, wherein the Nectin-4 antibody or the antigen binding fragment thereof comprises light chain complementarity determining regions (CDRs) comprising a sequence of amino acids as set forth in SEQ ID NOs: 7, 8, and 9 and heavy chain CDRs comprising a sequence of amino acids as set forth in SEQ ID NOs: 10, 11 and 12, and wherein the second ADC has a formula represented by:

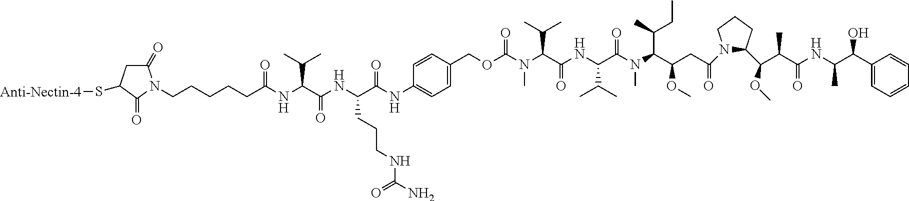

Embodiment 16: The method of any one of Embodiments 1 to 15, wherein the anti Nectin-4 antibody comprises a light chain comprising SEQ ID NO:15 and a heavy chain comprising SEQ ID NO:16.

Embodiment 17: The method of any one of Embodiments 1 to 16, wherein the anti Nectin-4 antibody is enfortumab.

Embodiment 18: The method of any one of Embodiments 1 to 17, wherein the Nectin-4 ADC is enfortumab vedotin (EV).

Embodiment 19: The method of any one of Embodiments 1 to 18, wherein the Trop-2 ADC is administered as an intravenous infusion on day 1 and day 8 of one or more 21-day treatment cycles.

Embodiment 20: The method of any one of Embodiments 1 to 19, wherein the Trop-2 ADC is administered at a dose of from 5 mg/kg to 10 mg/kg as an intravenous infusion on day 1 and day 8 of one or more 21-day treatment cycles.

Embodiment 21: The method of any one of Embodiments 1 to 19, wherein the Trop-2 ADC is administered at a dose of 7.5 mg/kg as an intravenous infusion on day 1 and day 8 of one or more 21-day treatment cycles.

Embodiment 22: The method of any one of Embodiments 1 to 19, wherein the Trop-2 ADC is administered at a dose of 8 mg/kg as an intravenous infusion on day 1 and day 8 of one or more 21-day treatment cycles.

Embodiment 23: The method of any one of Embodiments 1 to 19, wherein the Trop-2 ADC is administered at a dose of 10 mg/kg as an intravenous infusion on day 1 and day 8 of one or more 21-day treatment cycles.

Embodiment 24: The method of any one of Embodiments 1 to 23, wherein the Nectin-4 ADC is administered as an intravenous infusion on day 1 and day 8 of one or more 21-day treatment cycles.

Embodiment 25: The method of any one of Embodiments 1 to 23, wherein the Nectin-4 ADC is administered at a dose of from 0.5 mg/kg to 1.25 mg/kg as an intravenous infusion on day 1 and day 8 of one or more 21-day treatment cycles.

Embodiment 26: The method of any one of Embodiments 1 to 23, wherein the Nectin-4 ADC is administered at a dose of 1 mg/kg as an intravenous infusion on day 1 and day 8 of one or more 21-day treatment cycles.

Embodiment 27: The method of any one of Embodiments 1 to 23, wherein the Nectin-4 ADC is administered at a dose of 1.25 mg/kg as an intravenous infusion on day 1 and day 8 of one or more 21-day treatment cycles.

Embodiment 28: The method of any one of Embodiments 1 to 5, wherein the Trop-2 ADC is SG; and SG is administered at a dose of 5 mg/kg as an intravenous infusion on day 1 and day 8 of one or more treatment cycles; and the Nectin-4 ADC is EV; and EV is administered as an intravenous infusion at a dose 1 mg/kg as an intravenous infusion on day 1 and day 8 of one or more 21-day treatment cycles.

Embodiment 29: The method of any one of Embodiments 1 to 5, wherein the Trop-2 ADC is SG; and SG is administered at a dose of 5 mg/kg as an intravenous infusion on day 1 and day 8 of one or more treatment cycles; and the Nectin-4 ADC is EV; and EV is administered as an intravenous infusion at a dose 0.75 mg/kg as an intravenous infusion on day 1 and day 8 of one or more 21-day treatment cycles.

Embodiment 30: The method of any one of Embodiments 1 to 5, wherein the Trop-2 ADC is SG; and SG is administered at a dose of 5 mg/kg as an intravenous infusion on day 1 and day 8 of one or more treatment cycles; and the Nectin-4 ADC is EV; and EV is administered as an intravenous infusion at a dose 0.5 mg/kg as an intravenous infusion on day 1 and day 8 of one or more 21-day treatment cycles.

Embodiment 31: The method of any one of Embodiments 1 to 5, wherein the Trop-2 ADC is SG; and SG is administered at a dose of 7.5 mg/kg as an intravenous infusion on day 1 and day 8 of one or more treatment cycles; and the Nectin-4 ADC is EV; and EV is administered as an intravenous infusion at a dose 1.25 mg/kg as an intravenous infusion on day 1 and day 8 of one or more 21-day treatment cycles.

Embodiment 32: The method of any one of Embodiments 1 to 5, wherein the Trop-2 ADC is SG; and SG is administered at a dose of 8 mg/kg as an intravenous infusion on day 1 and day 8 of one or more treatment cycles; and the Nectin-4 ADC is EV; and EV is administered as an intravenous infusion at a dose 1.25 mg/kg on day 1 and day 8 of one or more 21-day treatment cycles.

Embodiment 33: The method of any one of Embodiments 1 to 5, wherein the Trop-2 ADC is SG; and SG is administered at a dose of 10 mg/kg as an intravenous infusion on day 1 and day 8 of one or more treatment cycles; and the Nectin-4 ADC is EV; and EV is administered as an intravenous infusion at a dose 1.25 mg/kg as an intravenous infusion on day 1 and day 8 of one or more 21-day treatment cycles.

Embodiment 34: The method of any one of Embodiments 1 to 5 and 19 to 27, wherein the Trop-2 ADC is SG and the Nectin-4 ADC is EV, wherein when the method is used to treat a population of subjects, the method achieves an overall response rate (ORR) of at least 60% at a median 14 month follow-up.

Embodiment 35: The method of any one of Embodiments 1 to 33, wherein the method is used to treat a population of subjects having advanced or metastatic UC, the method achieves an overall response rate (ORR) of at least 60%, at least 65%, or at least 70%.

Embodiment 36: The method of any one of claims 1 to 33, wherein the method is used to treat a population of subjects having advanced or metastatic UC, the method achieves an overall response rate (ORR) of 60-85%, 65-85%, 70-85%, 70-80%, or 70-75%.

Embodiment 37: The method of any one of Embodiments 1 to 36, wherein the subject has had a prior systemic therapy for metastatic urothelial carcinoma.

Embodiment 38: The method of any one of Embodiments 1 to 37, wherein the subject has metastatic urothelial carcinoma.

Embodiment 39: The method of Embodiment 38, wherein the subject has received prior platinum-based therapy for metastatic urothelial carcinoma, and the metastatic urothelial carcinoma has progressed during or after platinum-based therapy.

Embodiment 40: The method of Embodiment 38, wherein the subject has received prior immunotherapy for metastatic urothelial carcinoma, and the metastatic urothelial carcinoma has progressed during or after immunotherapy.

Embodiment 41: The method of Embodiment 38, wherein the subject has not had a prior systemic therapy for metastatic urothelial carcinoma.

Embodiment 42: The method of Embodiment 41, wherein the subject is ineligible for or refusing cisplatin based therapy.

Embodiment 43: The method of any one of Embodiments 1 to 42, wherein the method does not include concomitantly administering an additional systemic therapy.

Embodiment 44: The method of any one of Embodiments 1 to 42, wherein the method does not include administering to the subject an anti-PD-(L)1 antibody.

Embodiment 45: The method of any one of Embodiments 1 to 42, wherein the method further comprises administering to the subject an anti-PD-(L)1 antibody.

Embodiment 46: The method of Embodiment 45, wherein the anti-PD-(L)1 antibody is selected from the group consisting of pembrolizumab, nivolumab, cemiplimab, pidilizumab, spartalizumab, atezolizumab, avelumab, durvalumab, cosibelimab, sasanlimab, tislelizumab, retifanlimab, balstilimab, toripalimab, cetrelimab, genolimzumab, prolgolimab, lodapolimab, camrelizumab, budigalimab, avelumab, dostarlimab, envafolimab, sintilimab, and zimberelimab.

Embodiment 47: The method of Embodiment 45 or 46, wherein in the anti-PD-(L)1 antibody is pembrolizumab.

Embodiment 48: The method of Embodiment 47, wherein pembrolizumab is administered to the subject at a dose level of about 200 mg per dose.

Embodiment 49: The method of Embodiment 47 or 48, wherein pembrolizumab is administered once every 21-day treatment cycle.

Embodiment 50: The method of any one of Embodiments 47 to 49, wherein pembrolizumab is administered on day 1 of the one or more 21-day treatment cycles.

Embodiment 51: The method of Embodiment 47, wherein pembrolizumab is administered to the subject at a dose level of about 400 mg per dose.

Embodiment 52: The method of Embodiment 47 or 51, wherein pembrolizumab is administered once every 42-day treatment cycle.

Embodiment 53: The method of any one of Embodiments 47, 51, and 52, wherein pembrolizumab is administered on day 1 of the one or more 42-day treatment cycles.

Embodiment 54: The method of Embodiment 45 or 46, wherein the anti-PD-(L)1 antibody is zimberelimab.

Embodiment 55: The method of any one of Embodiment 45, 46, and 54, wherein zimberelimab is administered at a dose level in the range of 300 mg to 400 mg.

Embodiment 56: The method of any one of Embodiment 45, 46, and 54, wherein zimberelimab is administered at a dose level of about 360 mg.

Embodiment 57: The method of any one of Embodiment 45, 46, and 54-56, wherein zimberelimab is administered once every 21-day treatment cycle.

Embodiment 58: The method of any one of Embodiment 45, 46, and 54-57, wherein zimberelimab is administered on day 1 of the one or more 21-day treatment cycles.

Embodiment 59: The method of any one of claims 1 to 59, wherein when the method is used to treat a population of subjects, the method achieves a higher ORR compared to administering the Trop-2 ADC or the Nectin-4 ADC as a monotherapy to a population of subjects.

Embodiment 60: A method of improving clinical efficacy of either a SG monotherapy or a EV monotherapy in a population of human subjects having advanced or metastatic UC that has relapsed after, or is refractory to, platinum-based chemotherapy and/or immunotherapy, comprising administering to each subject in the population a combination therapy which comprises a therapeutically effective amount of SG and a therapeutically effective amount of EV.

Embodiment 61: The method of Embodiment 60, wherein the method achieves an ORR of at least 70%.

Embodiment 62: The method of Embodiment 60, wherein the method achieves an ORR of 70%-80%.

Embodiment 63: The method of any one of Embodiments 60 to 62, wherein the method achieves a median duration of response (DOR) of greater than 12 months.

Embodiment 64: The method of any one of claims 60 to 62, wherein the method achieves a median DOR greater than 14 months.

Embodiment 65: A method of achieving an improved durability of clinical response in a population of human subjects having advanced or metastatic UC that has relapsed after, or is refractory to, platinum-based chemotherapy and/or immunotherapy, compared to either a SG monotherapy or an EV monotherapy, comprising administering to each subject of the population of human subjects a combination therapy which comprises a therapeutically effective amount of SG and a therapeutically effective amount of EV.

Embodiment 66: The method of any one of Embodiments 60 to 65, wherein a 12-month progression free survival rate is at least 40%.

Embodiment 67: The method of any one of Embodiments 60 to 65, wherein a 12-month progression free survival rate is a 40%-80%.

Embodiment 68: The method of any one of Embodiments 60 to 65, wherein a 12-month progression free survival rate is a 40%-60%.

Embodiment 69: The method of any one of Embodiments 60 to 65, wherein a 12-month progression free survival rate is a 40%-50%.

Embodiment 70: The method of any one of Embodiments 60 to 65, wherein a 12-month progression free survival rate is a 40%-45%.

Embodiment 71: The method of any one of Embodiments 1 to 71, wherein the subject has not received prior EV treatment.

Embodiment 72: The method of any one of Embodiments 60 to 71, wherein the subject is cisplatin-ineligible.

Embodiment 73: The method of any one of Embodiments 60 to 71, wherein the subject is cisplatin-eligible.

Embodiment 74: A method of achieving a complete response in a human subject having mUC for at least approximately 6 months, comprising administering to the subject a combination therapy comprising a therapeutically effective amount of SG and a therapeutically effective amount of EV.

Embodiment 75: The method of any one of Embodiments 1 to 74, wherein when the method is used to treat a population of subjects, the method achieves less than about 20% serious treatment related adverse events (TRAEs).

Embodiment 76: The method of any one of Embodiments 1 to 74, wherein when the method is used to treat a population of subjects, the method achieves less than about 60% TRAEs of grade ≥3.

Embodiment 77: The method of any one of Embodiments 1 to 74, wherein when the method is used to treat a population of subjects, the method achieves less than about 70% TRAEs of grade ≥3.

Embodiment 78: The method of any one of Embodiments 1 to 74, wherein when the method is used to treat a population of subjects, the method achieves less than about 80% TRAEs of grade ≥3.

Embodiment 79: The method of any one of Embodiments 1 to 74, wherein when the method is used to treat a population of subjects, less than 20% subjects discontinue the therapy due to TRAE.

Embodiment 80: The method of any one of Embodiments 1 to 74, wherein when the method is used to treat a population of subjects, less than 30% subjects discontinue the therapy due to TRAE.

Embodiment 81: The method of any one of Embodiments 1 to 74, wherein when the method is used to treat a population of subjects, less than 40% subjects discontinue the therapy due to TRAE.

Embodiment 82: The method of any one of Embodiments 1 to 74, wherein when the method is used to treat a population of subjects, less than 50% subjects discontinue the therapy due to TRAE.

Embodiment 83: The method of Embodiment 2 or 3, wherein the safety or tolerability of the method is indicated by subjects' rates of discontinuation of treatment due to TRAE, and wherein subjects' rate of discontinuation is less than 20%, 30%, 40%, or 50%.

Embodiment 84: The method of any one of Embodiments 75 to 83, wherein the TRAE is selected from anemia, febrile neutropenia, chest pain—cardiac, supraventricular tachycardia, abdominal pain, diarrhea, enterocolitis, ileal obstruction, mucositis oral, nausea, small intestinal obstruction, fatigue, pain, abdominal infection, enterocolitis infectious, infections and infestations, sepsis, skin infection, upper respiratory infection, urinary tract infection, wound infection, lipase increased, lymphocyte count decreased, neutrophil count decreased, platelet count decreased, white blood cell decreased, anorexia, dehydration, hyperglycemia, hypoalbuminemia, hypokalemia, acute kidney injury, urinary tract obstruction, hiccups, pneumonitis, respiratory, thoracic and mediastinal disorders, hypertension, hypotension, and thromboembolic event.

Embodiment 85: The method of any one of Embodiments 60 to 84, wherein SG is administered at a dose of 7.5 mg/kg as an intravenous infusion on day 1 and day 8 of one or more 21-day treatment cycles; and EV is administered as an intravenous infusion at a dose 1.25 mg/kg on day 1 and day 8 of one or more 21-day treatment cycles.

Embodiment 86: The method of any one of Embodiments 60 to 84, wherein SG is administered at a dose of 8 mg/kg as an intravenous infusion on day 1 and day 8 of one or more 21-day treatment cycles; and EV is administered as an intravenous infusion at a dose 1.25 mg/kg on day 1 and day 8 of one or more 21-day treatment cycles.

Embodiment 87: The method of any one of Embodiments 60 to 84, wherein SG is administered at a dose of 10 mg/kg as an intravenous infusion on day 1 and day 8 of one or more 21-day treatment cycles; and EV is administered as an intravenous infusion at a dose 1.25 mg/kg on day 1 and day 8 of one or more 21-day treatment cycles.

Embodiment 88: A method of treating relapsed or refractory locally advanced or metastatic urothelial carcinoma in a population of human subjects, comprising administering to each subject of in the population a combination therapy which comprises: administering SG at a dose of 8 mg/kg as an intravenous infusion on day 1 and day 8 of one or more 21-day treatment cycles; and administering EV as an intravenous infusion at a dose 1.25 mg/kg on day 1 and day 8 of one or more 21-day treatment cycles; wherein the combination therapy achieves ORR of 65-75%.

Embodiment 89: A method of treating relapsed or refractory locally advanced or metastatic urothelial carcinoma in a population of human subjects, comprising administering to each subject of in the population a combination therapy which comprises: administering SG at a dose of 7.5 mg/kg as an intravenous infusion on day 1 and day 8 of one or more 21-day treatment cycles; and administering EV as an intravenous infusion at a dose 1.25 mg/kg on day 1 and day 8 of one or more 21-day treatment cycles; wherein the method achieves ORR of 65-75%.

Embodiment 90: The method of Embodiment 88 or 89, wherein combination therapy has a manageable safety profile.

Embodiment 91: The method of any one of Embodiment 88 to 90, wherein subjects' rates of discontinuation of the combination therapy due to TRAE is less than 20%, 30%, 40%, or 50%.

Embodiment 92: The method of any one of Embodiments 88-91, when the method is used to treat a population of subjects, the method achieves less than about 80% TRAEs of grade ≥3.

Embodiment 93: The method of Embodiment 91 or 92, wherein the TRAE is selected from anemia, febrile neutropenia, chest pain—cardiac, supraventricular tachycardia, abdominal pain, diarrhea, enterocolitis, ileal obstruction, mucositis oral, nausea, small intestinal obstruction, fatigue, pain, abdominal infection, enterocolitis infectious, infections and infestations, sepsis, skin infection, upper respiratory infection, urinary tract infection, wound infection, lipase increased, lymphocyte count decreased, neutrophil count decreased, platelet count decreased, white blood cell decreased, anorexia, dehydration, hyperglycemia, hypoalbuminemia, hypokalemia, acute kidney injury, urinary tract obstruction, hiccups, pneumonitis, respiratory, thoracic and mediastinal disorders, hypertension, hypotension, and thromboembolic event.

Embodiment 94: The method of any one of Embodiments 88 to 93, wherein combination therapy achieves a median duration of response (DOR) of greater than 12 months.

Embodiment 95: The method of any one of Embodiments 88 to 93, wherein combination therapy achieves a 12-month progression free survival (PFS) rate of at least 40%.

Embodiment 96: The method of any one of Embodiments 1 to 95, wherein the subject has received prophylactic granulocyte colony stimulating factor.

Embodiment 97: The method of any one of Embodiments 1 to 96, wherein the subject has received premedication comprising antipyretics, H1 blocker, and H2 blocker.

Embodiment 98: The method of Embodiment 97, wherein the antipyretics is acetaminophen; the H1 blocker is Benadryl or Loratidine; and the H2 blocker is Pepcid.

Embodiment 99: The method of Embodiment 98, wherein the subject has received acetaminophen 650 mg PO; Benadryl 25 mg PO/IV or Loratidine 10 mg PO; and H2 blocker Pepcid 20 mg PO/IV.

Embodiment 100: The method of any one of Embodiments 1 to 99, wherein the subject has received antiemetic premedication comprising a 5-HT3 inhibitor, an NK1-receptor antagonist, and dexamethasone.

Embodiment 101: The method of Embodiment 100, wherein the 5-HT3 inhibitor is ondansetron or palonosetron; and the NK1-receptor antagonist is fosaprepitant or aprepitant.

Embodiment 102: The method of any one of Embodiments 97 to 99, wherein the premedication is administered approximately 30 minutes before each SG administration.

Embodiment 103: The method of any one of Embodiments 28 to 102, EV and SG are administered in the order of EV, then SG.

Embodiment 104: The method of any one of Embodiments 47 to 53, EV, pembrolizumab, and SG are administered in the order of EV, pembrolizumab, then SG.

Embodiment 105: The method of any one of Embodiments 1 to 104, wherein SG is administered on day 1 and day 8 of at least about (11) 21-day treatment cycles.

Embodiment 106: The method of any one of Embodiments 1 to 104, wherein SG is administered on day 1 and day 8 of at least about (14) 21-day treatment cycles.

### Example 1

Phase 1 Clinical Trial—Dual-Antibody Drug Conjugate (DAD) Trial to Assess the Safety and Combination Maximum Tolerated Doses (MTDs) of SG in Combination with EV in Patients with Treatment Resistant mUC (NCT04724018)

This is an open-label, non-randomized phase I trial of combination SG and EV. Eligible patients had locally advanced or metastatic urothelial carcinoma (<50% variant histology, small cell excluded) and had progressed on platinum therapy and immunotherapy or had progressed on one line of therapy and were ineligible to receive cisplatin-based chemotherapy. Advanced disease was defined as unresectable, locally recurrent, or metastatic by AJCC 7^{th} edition. Other inclusion criteria included presence of measurable disease per Response Evaluate Criteria in Solid Tumors (RECIST) version 1.1, Eastern Cooperative Oncology Group (ECOG) performance status of ≤1, adequate organ function at baseline, and absence of untreated brain metastases.

### Inclusion Criteria

Patients met all of the inclusion criteria outlined below at screening/Day −1 were eligible for participation in this study:

The patients were 18 years of age or older and able to understand and give written informed consent.

Participants had histologically documented confirmed predominant urothelial carcinoma (i.e., of the bladder, renal pelvis, ureter or urethra). Patients with squamous differentiation or mixed cell types were eligible; small-cell carcinoma is not allowed. Patients with locally advanced unresectable disease are eligible.

Patients who were cisplatin eligible received prior treatment with platinum containing therapy defined as within the adjuvant/neoadjuvant setting with ≥ypT2 disease at surgery or recurrent or progressive disease within 12 months or receiving treatment with platinum in locally advanced or metastatic setting. In addition, they received a checkpoint inhibitor (CPI) in locally advanced or metastatic urothelial carcinoma setting. Patients who received CPI therapy in the neoadjuvant/adjuvant setting and had recurrent or progressive disease either during or within 3 months of therapy completion were eligible. A CPI is defined as a PD-1 or PD-L1 inhibitor.

Patients who were cisplatin-ineligible progressed on or since one line of therapy defined as therapy given in the adjuvant/neoadjuvant setting within 12 months of progression or receiving therapy for locally advanced or metastatic disease.

Patient progressed on or since most recent therapy.

The patients had an Eastern Cooperative Oncology Group (ECOG) performance status score of 0 or 1.

Participants had adequate organ and marrow function as defined below:

| | |
|---|---|
| Leukocytes | ≥3,000/mcL |
| Absolute neutrophil count | ≥1,500/mcL |
| Platelets | ≥100,000/mcL |
| Total bilirubin | ≤ institutional upper |
| | limit of normal (ULN) |
| AST(SGOT)/ALT(SGPT) | ≤2.5x institutional ULN |
| | OR |
| | ≤5x ULN with liver |
| | metastases and serum albumin |
| | >3 g/dL |
| Glomerular filtration | ≥30 mL/min/1.73 m2 |
| rate (GFR) | (by Cockcroft Gault formula) |

Human immunodeficiency virus (HIV)-infected participants on effective anti-retroviral therapy with undetectable viral load within 6 months were eligible for this trial.

Participants with a history of hepatitis C virus (HCV) infection had been treated and cured. For participants with HCV infection who were currently on treatment, they were eligible if they have an undetectable HCV viral load.

The patients had measurable disease by computed tomography (CT) or magnetic resonance imaging (MRI) as per response evaluation criteria in solid tumors version 1.1 (RECIST 1.1 criteria). Tumor lesions situated in a previously irradiated area were considered measurable if progression has been demonstrated in such lesions.

Female subjects of childbearing potential had a negative urine or serum pregnancy test within 72 hours prior to receiving the first dose of study medication. If the urine test was positive or could not be confirmed as negative, a serum pregnancy test was required.

Female subjects of childbearing potential were willing to use 2 methods of birth control or be surgically sterile or abstain from heterosexual activity for the course of the study through 6 months after the last dose of study medication. Subjects of childbearing potential are those who have not been surgically sterilized or have not been free from menses for >2 years.

Women of child-bearing potential and men must agree to use adequate contraception (hormonal or barrier method of birth control; abstinence) prior to study entry and for the duration of study participation. Should a woman become pregnant or suspect she is pregnant while she or her partner is participating in this study, she should inform her treating physician immediately. Men treated or enrolled on this protocol must also agree to use adequate contraception prior to the study, for the duration of study participation, and 4 months after completion of SG administration.

### Exclusion Criteria

Patients who met any of the exclusion criteria outlined below at screening/Day −1 were not eligible to be enrolled in this study:

Women who were pregnant or lactating. Pregnant women were excluded from this study because SG and EV have potential for teratogenic or abortifacient effects. Because there is an unknown but potential risk for adverse events in nursing infants secondary to treatment of the mother with EV or SG, breastfeeding should be discontinued if the mother is treated on protocol.

Patients had a prior anti-cancer biologic agent (including immune checkpoint inhibitors) within 4 weeks prior to Cycle 1 Day 1 (C1D1) or have had prior chemotherapy, targeted small molecule therapy, or radiation therapy within 2 weeks prior to C1D1. Subjects participating in observational studies were eligible.

Patients had any toxicities attributed to prior anti-cancer therapy that are not resolved to Grade 1 or baseline that could impose serious risk for complications before administration of study drug agent. If subjects received major surgery, they must have recovered adequately from the toxicity and/or complications from the intervention prior to starting therapy.

Patients who had previously received topoisomerase 1 inhibitors, SG or EV.

Patients who had an active second malignancy. Subjects with a history of malignancy that have been completely treated, with no evidence of active cancer for 3 years prior to start of therapy on trial (Cycle 1 Day 1 [C1D1]), or subjects with surgically-cured tumors with low risk of recurrence are allowed to enroll.

Patients who had known active central nervous system (CNS) metastases and/or carcinomatous meningitis. Subjects with previously treated brain metastases may participate provided they have stable CNS disease for at least 4 weeks prior to the first dose of study drug and all neurologic symptoms have returned to baseline, have no evidence of new or enlarging brain metastases, and are taking ≤20 mg/day of prednisone or its equivalent. All subjects with carcinomatous meningitis are excluded regardless of clinical stability.

Patients had active cardiac disease, defined as:
Myocardial infarction or unstable angina pectoris within 6 months prior to C1D1
History of serious ventricular arrhythmia (i.e., ventricular tachycardia or ventricular fibrillation), high-grade atrioventricular block, or other cardiac arrhythmias requiring anti-arrhythmic medications (except for atrial fibrillation that is well controlled with antiarrhythmic medication); history of QT interval prolongation
New York Heart Association (NYHA) Class III or greater congestive heart failure or left ventricular ejection fraction of <40%

Patients who had active chronic inflammatory bowel disease (ulcerative colitis, Crohn's disease) or gastrointestinal (GI) perforation within 6 months of C1D1.

Patients who had active serious infection requiring antibiotics (Contact sponsor PI or Co-PI for clarification).

Patients who had other concurrent medical or psychiatric conditions that, in the Investigator's opinion, may be likely to confound study interpretation or prevent completion of study procedures and follow-up examinations.

Patients who had high dose systemic corticosteroids (≥20 mg of prednisone or its equivalent) are not allowed within 2 weeks prior to C1D1.

Participants who were receiving any other investigational agents.

Patients who had history of allergic reactions attributed to compounds of similar chemical or biologic composition to EV or SG or any excipient contained in the drug formulations (including 2 (N morpholino) ethane sulfonic acid (MES), histidine, treahalose dihydrate polysorbate 80 and polysorbate 20).

Participants with uncontrolled intercurrent illness.

Participants with psychiatric illness/social situations that would limit compliance with study requirements.

Patients with uncontrolled diabetes. Uncontrolled diabetes is defined as hemoglobin A1C>8% or 7-8% with associated diabetes symptoms that are otherwise not explained.

Patients who had uncontrolled tumor related bone pain or impending spinal cord compression.

### Treatments

Eligible patients were planned to receive SG and EV intravenously at one of 4 different dose levels (DL) on days (D) 1 and 8 of a 21-day cycle with appropriate anti-emetic regimen and were treated until progression or unacceptable toxicity. Either drug could be held on D8 as needed for toxicity, but patients had to receive both drugs on D1 of each cycle until a protocol amendment was approved in March 2023, after which patients could continue monotherapy as needed for toxicity at any point after cycle 1 day 1 (C1D1). All drugs were given intravenously (IV). The starting doses were SG 8 mg/kg SG and EV 1 mg/kg (DL1). Then, dose escalation of EV at 1.25 mg/kg with SG at 8 mg/kg was established for DL2. Finally, dose escalation of both EV at 1.25 mg/kg and SG IV at 10 mg/kg was permitted, defining DL3. Dose de-escalation of SG to 6 mg/kg with the starting dose of EV 1 mg/kg (DL-1) was permitted if unacceptable DLTs were observed at DL1. Maximum weight for EV dose calculation was capped at 100 kg in all DLs; there was no weight cap for SG dosing. After observing 2 of the first 6 patients treated in DL1 developing neutropenic fever, prophylactic granulocyte colony stimulating factor (GCSF) was allowed at discretion of investigator starting at C1.

Dose reductions of EV and SG were permitted independently based on investigator assessment of toxicities. Permitted EV dose reductions were as follows: (−1) 1 mg/kg, (−2) 0.75 mg/kg, and (−3) 0.5 mg/kg. SG dose reductions varied based on initial dose; those starting at 10 mg/kg could be reduced to 7.5 mg/kg and then 5 mg/kg. Those starting at 8 mg/kg could be reduced to 6 mg/kg and 5 mg/kg while those starting at 6 mg/kg had one dose reduction to 5 mg/kg.

### Study Design

The study employed a Bayesian Optimal Interval (BOIN) design to estimate the maximum tolerated dose (MTD) of the combination of SG and EV. In brief, assuming a target DLT rate of 30%, the decision for dose escalation or de-escalation was made for each cohort of 3 patients as follows: if the observed DLT rate at the current dose level was ≤0.236, then the next cohort was treated at the next higher dose level; if it was ≥0.359, then the next cohort was treated at the next lower dose level; if the DLT rate fell between 0.236 and 0.359, the dose level was retained (Table 13). The first patient enrolled at a new dose level was required to complete one full cycle (21 days) before additional patients could be enrolled in that cohort. Study would halt enrollment once either 18 patients enrolled in a single DL or a total of 24 patients enrolled overall.

### Dose Modifications for Adverse Reactions

SG and EV usage may be withheld, modified, or discontinued to manage adverse reactions as described in Tables 4-7.

TABLE 4
Recommended Dose Reduction Schedule for non-hematologic toxicities of SG
Non-Hematologic Toxicity

| | | |
|---|---|---|
| Grade 4 non-hematologic toxicity of any duration, | First | 25% reduction |
| OR | | *NOTE: if a patient is receiving |
| Any grade 3-4 nausea, vomiting or diarrhea due to | | starting SG dose of 6 mg/kg, a |
| treatment | | single reduction to 5 mg/kg is |
| that is not controlled with antiemetics and anti- | | the only reduced dose allowed. |
| diarrheal | | If a second occurrence, must |
| agents | | discontinue. |
| OR | Second | 50% reduction |
| Other grade 3-4 non-hematologic toxicity persisting | Third | Discontinue Treatment |
| >48 hours despite optimal medical management, | | |
| OR | | |
| At time of scheduled treatment, grade 3-4 non- | | |
| hematologic toxicity, which delays dose by 2 or 3 | | |
| weeks for recovery to ≤ grade 1 | | |
| In the event of grade 3-4 non-hematologic toxicity, | First | Discontinue Treatment |
| which does not recover to ≤ grade 1 within 3 weeks | | |

*Up to 2 dose level reductions are allowed with minimum dose of 5 mg/kg; if a patient isreceiving 6 mg/kg, a single reduction to 5 mg/kg is the only reduced dose allowed
**The reduced doses for SG include the following for the different dose levels: 10 mg/kg → 7.5 mg/kg → 5 mg/kg, 8 mg/kg → 6 mg/kg →5 mg/kg and 6 mg/kg → 5 mg/kg

TABLE 5
Dose modifications for EV associated nonhematologic toxicity

| Adverse | | |
|---|---|---|
| Reaction | Severity* | Dose Modification* |
| Hyperglycemia | Blood | Withhold until elevated blood glucose |
| | glucose | has improved to ≤250 mg/dL, then |
| | >250 | resume treatment at the same dose level. |
| | mg/dL | |
| Peripheral | Grade 2 | Withhold until Grade ≤1, then resume |
| Neuropathy | | treatment at the same dose level (if first |
| | | occurrence). For a recurrence, withhold |
| | | until Grade ≤1 then, resume treatment |
| | | reduced by one dose level*. |
| | Grade ≥3 | Permanently discontinue. |
| Skin Reactions | See Table 6 | |
| Other | Grade 3 | Withhold until Grade ≤1, then resume |
| nonhematologic | | treatment at the same dose level or |
| Toxicity | | consider dose reduction by one dose level |
| | Grade 4 | Permanently discontinue. |

Dose levels of EV are as follows: 1.25 mg /kg (maximum of 125 mg) → 1 mg/kg (maximum of 100 mg) → 0.75 mg/kg (maximum of 75 mg) → 0.5 mg/kg (maximum of 50 mg); Up to 3 dose level reductions are allowed

TABLE 6
Dose modifications for skin reactions
Any Grade
For suspected Stevens-Johnson syndrome (SJS), suspected toxic epidermal necrolysis (TEN), or
bullous lesions, immediately withhold EV and refer the subject to a dermatologist/specialist for
diagnosis and specialized care.
For confirmed SJS or TEN, permanently discontinue treatment.
If SJS or TEN is ruled out, see recommendations provided below for skin reactions.

| Grade 1 | Grade 2 | Grade 3 | Grade 4 |
|---|---|---|---|
| For Grade 1 rash or | Continue at same dose | For Grade 3 rash or | For confirmed SJS or |
| skin reactions, may | level. | skin reactions withhold | TEN, or Grade 4 rash, |
| continue at same dose | For worsening rash or | enfortumab vedotin | permanently |
| level. See also section | skin reactions | until toxicity is ≤ | discontinue enfortumab |
| on management of | or skin reactions | Grade 1 or has returned | vedotin. |
| rash. | with concomitant | to baseline, then | |
| | fever, withhold | resume treatment at the | |
| | consider withholding | same dose level or | |
| | enfortumab vedotin | consider dose reduction | |
| | until toxicity is ≤ | by 1 level. | |
| | Grade 1 or has returned | Consider referral of the | |
| | to baseline, then | subject to a | |
| | resume treatment at the | dermatologist/specialist | |
| | same dose level or | for diagnosis and | |
| | consider dose reduction | specialized care. | |
| | by 1 level. Consider | See also section on | |
| | referral of the subject | management of rash. | |
| | to a dermatologist/ | For suspected SJS or | |
| | specialist for diagnosis | suspected TEN | |
| | and specialized care. | withhold enfortumab | |
| | See also section on | vedotin. Consider | |
| | management of rash. | referral of the subject | |
| | | to a | |
| | | dermatologist/specialist | |
| | | for diagnosis and | |
| | | specialized care. | |
| | | Subjects who have | |
| | | confirmed SJS or | |
| | | recurrent Grade 3 rash | |
| | | events should have | |
| | | enfortumab vedotin | |
| | | permanently | |
| | | discontinued. | |

TABLE 7
Dose Modifications for Hematologic Toxicity

| Adverse Reaction: Severe Neutropenia | Occurrence | Dose Modification |
|---|---|---|
| Grade 4 neutropenia ≥7 days, | First | Reduce SG dose 1 level |
| OR | Second | Reduce SG dose 2 levels |
| Grade 3 febrile neutropenia (absolute neutrophil | | *NOTE: if a patient is |
| count <1000/mm3 and fever ≥38.5° C.), | | receiving starting SG dose of |
| OR | | 6 mg/kg, a single reduction to |
| At time of scheduled treatment, Grade 3-4 | | 5 mg/kg is the only reduced |
| neutropenia which delays dosing by 2 or 3 weeks for | | dose allowed. |
| recovery to ≤ Grade 1 | Third+ | Reduce EV by 1 dose level |
| | | (EV may undergo up to 3 |
| | | dose reductions, dependent |
| | | upon starting dose level) |
| | | *If recurs when SG and EV |
| | | each at lowest dose level, |
| | | must discontinue SG and |
| | | continue EV alone. |
| At time of scheduled treatment, Grade 3-4 | First | Discontinue Study Treatment |
| neutropenia which delays dosing beyond 3 weeks | | |
| for recovery to ≤ Grade 1 | | |
| Grade 2 Thrombocytopenia | First | Hold SG and EV until |
| | | resolution |
| | Persists >7 | Decrease SG and EV 1 dose |
| | days | level |
| Other Non-Neutropenic Hematologic Toxicities | First | Reduce SG and EV 1 dose |
| | | level |
| At time of scheduled treatment, grade 3-4 non- | | Reduce SG and EV 2 dose |
| neutropenic hematologic toxicity which delays dose | | levels |
| by 2 or 3 weeks for recovery to ≤ grade 1 | | *NOTE: if a patient is |
| | | receiving starting SG dose of |
| | | 6 mg/kg, a single reduction to |
| | | 5 mg/kg is the only reduced |
| | | dose allowed. If a second |
| | | occurrence, reduce EV by 2^{nd} |
| | | dose level. |
| | Third | Discontinue Study Treatment |
| In the event of grade 3-4 non-neutropenic | First | Discontinue Study Treatment |
| hematologic toxicity which does not recover to | | |
| ≤ grade 1 within 3 weeks | | |

*Up to 2 dose level reductions of SG are allowed with minimum dose of 5 mg/kg; if a patient is receiving 6 mg/kg, a single reduction to 5 mg/kg is the only reduced dose allowed; thus, the reduced doses for SG include the following for the different dose levels: 10 mg/kg → 7.5 mg/kg → 5 mg/kg, 8 mg/kg → 6 mg/kg → 5 mg/kg and 6 mg/kg → 5 mg/kg; Up to 3 dose reductions of EV may be instituted as follows: 1.25 mg/kg (maximum of 125 mg) → 1.0 mg/kg (maximum of 100 mg) → 0.75 mg/kg (maximum of 75 mg) → 0.5 mg/kg (maximum of 50 mg); granulocyte-colony stimulating factor (G-CSF) or equivalent may be administered after cycle 1 per investigator discretion in addition to or instead of dose reductions based on ASCO guidelines.

### Statistical Analysis

The primary objective was to evaluate the feasibility and toxicities of SG and EV by estimation of MTD in combination. Treatment toxicity was assessed by Common Terminology Criteria for Adverse Events (CTCAE) version 5.0 with monitoring up to 4 weeks after the last dose of therapy. Treatment related adverse events (TRAEs) are defined as AEs assessed by the investigator as at least possibly related to study treatment. Clinical and laboratory evaluations were performed on days 1 and 8 of each cycle. DLTs were assessed during cycle 1 of therapy and defined by ≥1 of the following: neutropenic fever, thrombocytopenic bleeding, grade 3 neuropathy of any duration, and any other grade ≥3 non-hematologic toxicity including hyperglycemia (except alopecia) lasting >1 week or requiring >3 weeks interruption of therapy or dose reduction. Upon trial completion, MTD was determined using isotonic estimate of observed toxicity probability obtained by the pooled-adjacent-violators algorithm within the BOIN design. The recommended phase 2 dose (RP2D) was selected based on an assessment of the MTD during cycle 1 taking into consideration clinical judgement and cumulative toxicities observed in subsequent cycles.

Secondary objectives were to assess objective response rate (ORR), progression-free survival (PFS), and overall survival (OS). ORR was defined as the proportion of patients with complete response (CR) or partial response (PR) per RECIST version 1.1, as determined by the investigator.¹⁸ PFS was defined as time from treatment initiation to disease progression by RECIST criteria, death from any cause, or censored at the date of last disease assessment. OS was defined as the time from treatment initiation to death or censored at the date of last follow-up for those who had not died. Radiographic imaging was performed at baseline and every 6 weeks for the first 4 cycles, followed by every 9 weeks thereafter. ORR was summarized descriptively. The distributions for PFS and OS were estimated using the Kaplan-Meier method. Evaluable patients for primary and secondary endpoint analyses included those who received at least one dose of either of study treatment. The study was not powered for efficacy endpoints.

### Results

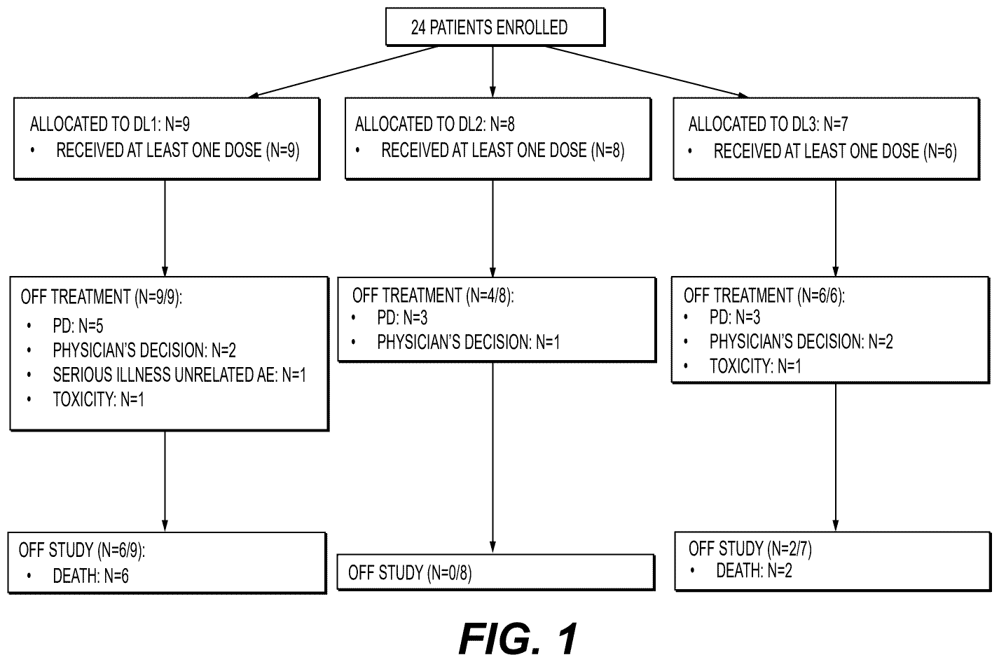

Between May 2021 and April 2023, a total of 24 patients were enrolled across 3 dose levels from Dana-Farber Cancer Institute and Massachusetts General Hospital; one patient never started therapy on protocol and was excluded from the analysis (FIG. 1). The median age was 70 years (range 41-88). 22/23 had received at least 2 lines of therapy; only one patient had received immunotherapy alone. 26% of patients had liver metastases (Table 8). Nine patients were enrolled in DL1, 8 patients in DL2, and 7 patients in DL3, one of whom never initiated therapy and was consequently excluded from the analysis. The last 2 patients in the final cohort were not escalated to DL3 given concerns for toxicity and responses already demonstrated with lower doses of SG and EV (FIG. 1). Starting doses and reductions of SG and EV are outlined in Table 9. Out of the 18 patients who received prophylactic GCSF during their treatment, 14 patients received it on C1D8. As of the data cutoff date in July 2023, the median follow-up was 14 months and 4 patients remain on therapy (FIG. 1). Eleven patients discontinued for disease progression and there was one death on trial prior to first imaging assessment in DL3. Five patients elected to pursue treatment break in the absence of disease progression at investigator discretion given concern for cumulative toxicity in the setting of durable response (after 11-18 cycles), while one patient discontinued for intolerable grade 2 neuropathy after 18 cycles. (FIG. 1).

At time of data cutoff on Apr. 8, 2024, a median follow-up was 22 months (range up to 34 months). The median number of cycles administered for SG and EV was 11 (range 1-22+). ORR was 70% (16/23, 95% Confidence interval (CI):47-87%) with only 3 pts experiencing progressive disease as their best response; 7 pts experienced >70% shrinkage of which 4 were complete responses. Median duration of response was 10 months (range 3-33+) with ongoing responses in 6 pts. Median PFS was 8.9 months (95% CI: 4.8-11.8). 12-month OS rate was 78% (95% CI: 55-90). There were no new safety signals or unexpected delayed toxicities.

With a median follow-up of 22 months, SG+EV continues to show encouraging activity with no new safety signals and with ORR of 70% in patients with treatment resistant mUC. This data supports the ongoing expansion cohorts of the SG 7.5 mg/kg and EV 1.25 mg/kg D1, 8 every 21 days in the treatment-resistant setting and in combination with pembrolizumab as first-line therapy for cisplatin-ineligible mUC (NCT04724018).

### Safety

Among 9 patients treated in DL1 there were 2 DLTs (1 grade 3 febrile neutropenia and 1 grade 4 sepsis in setting of no prophylactic GCSF). In 8 patients treated at DL2, there was one DLT (delay of C2D1>3 weeks due to a delayed autoimmune colitis attributed to prior pembrolizumab therapy). In DL3, 3 of 6 patients who received 1 dose of therapy experienced a DLT (1 grade 3 febrile neutropenia, 1 delay of treatment >3 weeks, 1 grade 3 mucositis). Subsequently, the combination MTD was estimated at DL3 with SG 10 mg/kg and EV 1.25 mg/kg (with a maximum dose 125 mg) days 1 and 8 of a 21-day cycle using the combination of number of patients and DLTs at each DL. Details regarding the selection of the MTD, posterior DLT estimates with a corresponding 85% credible interval along with probability that the toxicity rate for each doses exceeds the target rate of 30% are provided in table 10. However, due to observed cumulative toxicities at DL3, the RP2D was selected at DL2 (SG 8 mg/kg and EV 1.25 mg/kg (maximum dose 125 mg) D1,8 of 21-day cycle).

78% (18/23) of patients experienced grade ≥3 AE regardless of attribution at any DL and maximum grade 3-5 adverse events regardless of attribution are summarized according to dose level in Table 14. The most common TRAEs of any grade occurring during the trial included diarrhea, anemia, neutropenia, fatigue, alopecia, neuropathy and nausea. Most common TRAE≥grade 3 included neutropenia (8/23; 35%), anemia (8/23; 35%), urinary tract infection (3/23; 13%), fatigue (2/23; 9%) and diarrhea (2/23; 9%) across all DL (Table 11). There was one death on study in a patient at DL3 (pneumonitis) within 30 days of C1D8 possibly related to EV. Dose reductions of EV and SG were required in 48% (11/23) of patients for TRAEs; in addition, only 2 patients discontinued protocol therapy due to toxicity (1 neuropathy, 1 pneumonitis). Notably, neuropathy (57%) and diarrhea (87%) of any grade were the most common treatment-related adverse events that necessitated dose reductions of EV and SG respectively.

### Efficacy

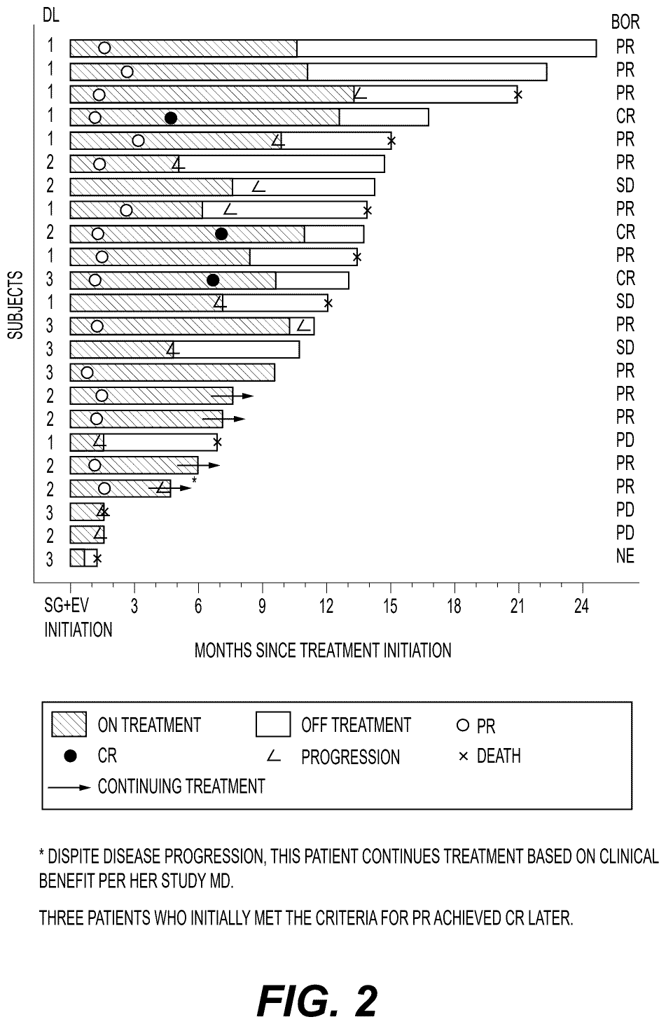

Among 23 patients evaluated for response, the ORR including confirmed and unconfirmed responses was 70% (95% CI 47-87%), only 3 patients (13%) had PD as the best response (Table 12). Nine patients remain without evidence of disease progression including 6 of 8 who stopped therapy for reasons other than disease progression and have continued stability off treatment up to 15 months (FIG. 2). With immature median follow-up of 14 months, the 12-month PFS and OS rates were 41% (95% CI: 18-62) and 86% (95% CI: 61-95), respectively.

### Discussion

The estimated MTD was the one using full dose of both SG and EV agents on days 1,8 of a 21-day cycles. However, in the present trial, the RP2D was determined by examining the MTD and using clinical judgement accounting for cumulative toxicities across repetitive cycle given that the MTD is determined in the first cycle only. The determined RP2D was SG 8 mg/kg with EV 1.25 mg/kg (max dose 125 mg) IV on D1, 8 every 21 days for future studies. When combined at full doses, 3 of 6 patients experienced DLTs within the first cycle and one patient experienced a possibly treatment related death within 32 days of C1D8.

The ability to combine two standard therapies in urothelial carcinoma in the treatment resistant setting is noteworthy as a proportion of patients with urothelial carcinoma are not able to be treated in second line and beyond. As an example, in the SEER data base meta-analysis, among chemotherapy-treated patients, 61% had only one line of therapy while 25% went on to receive two lines of therapy and only 14% had 3 lines of therapy or more. As such, combinations of agents in the second- or third-line setting may ensure greater receipt active therapies.

The responses seen are more frequent than with any ADC monotherapy in the treatment resistant setting. While patients could have progressed on only one line of therapy, 22 of 23 patients had received both platinum and immunotherapy prior to enrolling on trial. In phase 2 trials, patients treated with either SG or EV after immunotherapy alone have ORR of 32% and 52% respectively while patients receiving either SG or EV following platinum and immunotherapy have ORR of 28% and 44% making the ORR in the combination therapy of 70% notable. A trial combining combination versus sequential therapy is probably not feasible due to the high dropout rate of patients in the subsequent line of therapy in metastatic bladder cancer. More importantly, while early, there appears to be a clinically significant durability of response, with 6 of 9 responses lasting for a median of over 12 months. By comparison the median PFS in phase 2 trials of monotherapy with either SG or EV is less than 6 months.

The patient population is representative of patients with mUC seen in the clinic. The median age is 70 with a range up to 88 years similar to the patient population studied in cohort B of EV-201 that established EV monotherapy as an option for patients progressing on immunotherapy. The non-overlapping toxicities of these two ADCs allowed for administration of effective combination doses in the present trial, and the overall toxicity in this study compares favorably to monotherapy where over 50% of patients experience grade 3 TRAEs. With the use of prophylactic GCSF in those patients over age 70 or at risk for febrile neutropenia this risk was minimized. There was one treatment related death on study with a patient in DL3 experiencing pneumonitis; while exact etiology remains unclear, patient did have a preceding infection with concomitant long term amiodarone use. Ultimately, this was felt to be possibly related to EV with reports of grade 5 pneumonitis in other EV monotherapy trials. Given no other synergistic toxicity signals, it is not felt to be related to addition of SG. It is also important to note that of the remaining patients, there were no discontinuations for unacceptable toxicity within the first 6 months of therapy.

Patients enrolled on this protocol are representative of those patients with mUC in other trials and in the community; 22/23 had received at least 2 lines of therapy with a median age of 70. Second, the optimal duration of therapy remains uncertain. Treatment discontinuation was not built into the protocol and patients had to initiate both drugs on D1 of each cycle to remain on protocol until a study amendment was activated in March 2023 (22 months after trial activation.) Treatment discontinuation of EV due to cumulative neuropathy was seen in 2 patients after 13 and 15 cycles respectively while 4 other patients discontinued therapy for cumulative toxicities in setting of impressive response after 10-15 cycles. None of these patients has progressed with time off treatment ranging for over 15 months.

TABLE 8
Baseline demographic and disease characteristics.

| | |
|---|---|
| N | 23 |
| Age, years, median (IQR), [range] | 70 (63-76), [41-88] |
| Sex | |
| Female | 5 (22%) |
| Male | 18 (78%) |
| Race/Ethnicity | |
| White | 19 (83%) |
| Asian | 2 (9%) |
| Hispanic or Latino | 2 (9%) |
| ECOG performance status | |
| 0 | 14 (61%) |
| 1 | 9 (39%) |
| Primary site | |
| Bladder | 16 (70%) |
| Urethra | 1 (4%) |
| Upper tract | 6 (26%) |
| Histology | |
| Mixed urothelial | 7 (30%) |
| Pure urothelial | 16 (70%) |
| Cisplatin eligibility | |
| Eligible | 19 (83%) |
| Ineligible | 4 (17%) |
| Number of lines of prior therapy | |
| 1 | 1 (4%) |
| 2 | 11 (48%) |
| 3-5 | 11 (48%) |
| Prior therapy | |
| Immunotherapy* | 22 (96%) |
| Cisplatin-based chemotherapy | 18 (78%) |
| Carboplatin-based chemotherapy | 6 (26%) |
| Metastasic sites | |
| Bone | 6 (26%) |
| Kidney | 3 (13%) |
| Liver | 6 (26%) |
| Lung | 5 (22%) |
| Lymph nodes | 17 (74%) |

Values are n (%) or median (IQR).
IQR = interquartile range;
ECOG = Eastern Cooperative Oncology Group.
*Immunotherapy includes avelumab, pembrolizumab, and nivolumab.

TABLE 9
Treatment summary according to dose level, as of July 2023 data cutoff.

| | | Median number | Starting dose | | Number patients and dose | | Number patients and dose | |
|---|---|---|---|---|---|---|---|---|
| | Number | cycles initiated† | SG | EV | with one dose reduction | | with two dose reductions | |
| | patients | (range) | (mg/kg) | (mg/kg) | SG | EV | SG | EV |
| DL1 | 9 | 12 (2-19) | 8 | 1 | 4 | 1 | 1 | 3 |
| | | | | | (6 mg/kg) | (0.75 mg/kg) | (5 mg/kg) | (0.5 mg/kg) |
| DL2 | 8 | 10 (2-15) | 8 | 1.25 | 2 | 2 | 0 | 1 |
| | | | | | (6 mg/kg) | (1 mg/kg) | (5 mg/kg) | (0.75 mg/kg) |
| DL3 | 6 | 9 (1-16) | 10 | 1.25 | 3 | 3 | 1 | 1 |
| | | | | | (7.5 mg/kg) | (1 mg/kg) | (5 mg/kg) | (0.75 mg/kg) |

SG = Sacituzumab govitecan; EV = Enfortumab vedotin-ejfv; DL = dose level.
The maximum weight for dose calculation is capped at 100 kg for EV.
†The initiation of a treatment cycle is considered when both SG and EV were administered up until March 2023, with the option for either administration after cycle 1 allowed starting from March 2023 onwards.

TABLE 10
BOIN estimates for maximum tolerated dose selection.

| | Number of patients | Number of DLT | Posterior % DLT estimate | Probability |
|---|---|---|---|---|
| | treated | (%) | (95% Credible Interval) | (DLT >0.3ldata) |
| DL1 | 9 | 2 (22%) | 17% (2-46) | 0.17 |
| DL2 | 8 | 1 (13%) | 17% (2-46) | 0.17 |
| DL3 | 6 | 3 (50%) | 50% (15-85) | 0.84 |

DL = dose level;
DLT = dose limiting toxicity.

TABLE 11
Summary of treatment related adverse events observed in >10%* of 23 patients treated at any
dose level, as of July 2023 data cutoff.

| | Grade 1 | Grade 2 | Grade 3 | Grade 4 |
|---|---|---|---|---|
| Diarrhea | 12 (52.2%) | 6 (26.1%) | 2 (8.7%) | — |
| Anemia | 4 (17.4%) | 5 (21.7%) | 8 (34.8%) | — |
| Neutrophil count decreased | 3 (13.0%) | 5 (21.7%) | 4 (17.4%) | 4 (17.4%) |
| Fatigue | 8 (34.8%) | 5 (21.7%) | 2 (8.7%) | — |
| Alopecia | 3 (13.0%) | 11 (47.8%) | — | — |
| Peripheral sensory neuropathy | 5 (21.7%) | 8 (34.8%) | — | — |
| Nausea | 7 (30.4%) | 2 (8.7%) | 1 (4.3%) | — |
| Alanine aminotransferase increased | 8 (34.8%) | 2 (8.7%) | — | — |
| Aspartate aminotransferase increased | 9 (39.1%) | 1 (4.3%) | — | — |
| Mucositis | 2 (8.7%) | 6 (26.1%) | 1 (4.3%) | — |
| Weight loss | 3 (13.0%) | 4 (17.4%) | — | — |
| Alkaline phosphatase increased | 6 (26.1%) | 1 (4.3%) | — | — |
| Rash maculo-papular | 6 (26.1%) | 1 (4.3%) | — | — |
| Dry eye | 4 (17.4%) | 2 (8.7%) | — | — |
| Constipation | 5 (21.7%) | 1 (4.3%) | — | — |
| Dysgeusia | 5 (21.7%) | 1 (4.3%) | — | — |
| Watering eyes | 5 (21.7%) | 1 (4.3%) | — | — |
| Hypomagnesemia | 4 (17.4%) | 1 (4.3%) | — | — |
| Pruritus | 1 (4.3%) | 4 (17.4%) | — | — |
| Hypophosphatemia | 1 (4.3%) | 3 (13.0%) | — | — |
| Urinary tract infection | — | 1 (4.3%) | 3 (13.0%) | — |
| Anorexia | 2 (8.7%) | 1 (4.3%) | 1 (4.3%) | — |

*One grade 5 pneumonitis, possibly related to Enfortumab vedotin-ejfv, is not included in the table.

TABLE 12
Objective response in overall cohort and across dose levels,
as of July 2023 data cutoff.

| | Overall | DL1 | DL2 | DL3 |
|---|---|---|---|---|
| | (N = 23) | (N = 9) | (N = 8) | (N = 6) |
| Objective Response | 70 (47-87) | 78 (40-97) | 75 (35-97) | 50 (12-88) |
| Rate, % (95% CI) | | | | |
| Best Overall | | | | |
| Response | | | | |
| CR | 3 | 1 | 1 | 1 |
| PR | 13 | 6 | 5 | 2 |
| SD | 3 | 1 | 1 | 1 |
| PD | 3 | 1 | 1 | 1 |
| NE | 1 | 0 | 0 | 1 |
| Total | 23 | 9 | 8 | 6 |

DL = dose level;
CI = confidence interval;
IQR = Interquartile range;
CR = complete response;
PR = partial response;
SD = stable disease;
PD = progressive disease;
NE = not evaluated.

TABLE 13
Dose escalation, de-escalation using the BOIN design.

| | # of patients treated at current dose level | | | | | | | | | | | | | | | | | |
|---|---|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 | 17 | 18 |
| Escalate | 0 | 0 | 0 | 0 | 1 | 1 | 1 | 1 | 2 | 2 | 2 | 2 | 3 | 3 | 3 | 3 | 4 | 4 |
| If # of DLT≤ | | | | | | | | | | | | | | | | | | |
| Retain | | | 1 | | | 2 | | | 3 | | | 3-4 | | | 4-5 | | | 5-6 |
| if # of DLT= | | | | | | | | | | | | | | | | | | |
| De-escalate | 1 | 1 | 2 | 2 | 2 | 3 | 3 | 3 | 4 | 4 | 4 | 5 | 5 | 6 | 6 | 6 | 7 | 7 |
| if # of DLT≥ | | | | | | | | | | | | | | | | | | |
| Eliminate dose | NA* | NA | 3 | 3 | 4 | 4 | 5 | 5 | 5 | 6 | 6 | 7 | 7 | 8 | 8 | 8 | 9 | 9 |
| if # of DLT≥ | | | | | | | | | | | | | | | | | | |

Assuming a 30% target toxicity rate, the dose escalation and de-escalation boundaries are 0.24 and 0.36, respectively.
*A dose cannot be eliminated before treating 3 patients.
Ying Yuan and Suyu Liu (2021). BOIN: Bayesian Optimal INterval (BOIN) Design for Single-Agent and Drug- Combination Phase I Clinical Trials. R package version 2.7.2.

TABLE 14
Grade 3-5 adverse events regardless of attribution,
according to dose level, as of July 2023 data cutoff.

| | Dose level 1 | | Dose level 2 | | Dose level 3 | | |
|---|---|---|---|---|---|---|---|
| CTCAE v5.0 | Grade 3 | Grade 4 | Grade 3 | Grade 4 | Grade 3 | Grade 4 | Grade 5 |
| N patients | 9 | | 8 | | 6 | | |
| analyzed, N = 23 | | | | | | | |
| Max grade per | 5 | 2 | 5 | 1 | 2 | 2 | 1 |
| patient | | | | | | | |
| Anemia | 3 (33.3) | — | 1 (12.5) | — | 4 (66.7) | — | — |
| Febrile neutropenia | 1 (11.1) | — | — | — | 1 (16.7) | — | — |
| Chest pain - cardiac | 1 (11.1) | — | — | — | — | — | — |
| Supraventricular | 1 (11.1) | — | — | — | — | — | — |
| tachycardia | | | | | | | |
| Abdominal pain | 1 (11.1) | — | 1 (12.5) | — | 1 (16.7) | — | — |
| Diarrhea | 2 (22.2) | — | 1 (12.5) | — | — | — | — |
| Enterocolitis | — | — | 1 (12.5) | — | — | — | — |
| Ileal obstruction | 1 (11.1) | — | — | — | — | — | — |
| Mucositis oral | — | — | — | — | 1 (16.7) | — | — |
| Nausea | 1 (11.1) | — | — | — | — | — | — |
| Small intestinal | 1 (11.1) | — | 1 (12.5) | — | — | — | — |
| obstruction | | | | | | | |
| Fatigue | — | — | 1 (12.5) | — | 1 (16.7) | — | — |
| Pain | — | — | 1 (12.5) | — | — | — | — |
| Abdominal | 1 (11.1) | — | — | — | — | — | — |
| infection | | | | | | | |
| Enterocolitis | 1 (11.1) | — | — | — | — | — | — |
| infectious | | | | | | | |
| Infections and | 1 (11.1) | — | — | — | 2 (33.3) | — | — |
| infestations - Other, | | | | | | | |
| specify | | | | | | | |
| Sepsis | — | 1 (11.1) | — | — | — | — | — |
| Skin infection | 1 (11.1) | — | — | — | — | — | — |
| Upper respiratory | 2 (22.2) | — | — | — | — | — | — |
| infection | | | | | | | |
| Urinary tract | 1 (11.1) | — | — | — | 2 (33.3) | — | — |
| infection | | | | | | | |
| Wound infection | 1 (11.1) | — | — | — | — | — | — |
| Lipase increased | — | 1 (11.1) | — | — | — | — | — |
| Lymphocyte count | 1 (11.1) | — | — | 1 (12.5) | — | — | — |
| decreased | | | | | | | |
| Neutrophil count | 3 (33.3) | 1 (11.1) | — | — | 1 (16.7) | 3 (50.0) | — |
| decreased | | | | | | | |
| Platelet count | — | — | — | 1 (12.5) | — | — | — |
| decreased | | | | | | | |
| White blood cell | 1 (11.1) | — | — | — | 2 (33.3) | — | — |
| decreased | | | | | | | |
| Anorexia | — | — | — | — | 1 (16.7) | — | — |
| Dehydration | — | — | — | — | 1 (16.7) | — | — |
| Hyperglycemia | — | — | — | — | 1 (16.7) | — | — |
| Hypoalbuminemia | — | — | 1 (12.5) | — | 1 (16.7) | — | — |
| Hypokalemia | 1 (11.1) | — | — | — | — | — | — |
| Acute kidney injury | 1 (11.1) | — | — | — | 1 (16.7) | — | — |
| Urinary tract | — | — | — | — | 1 (16.7) | — | — |
| obstruction | | | | | | | |
| Hiccups | — | — | — | — | 1 (16.7) | — | — |
| Pneumonitis | — | — | — | — | — | — | 1 (16.7) |
| Respiratory, | — | — | — | — | 1 (16.7) | — | — |
| thoracic and | | | | | | | |
| mediastinal | | | | | | | |
| disorders - Other, | | | | | | | |
| specify | | | | | | | |
| Hypertension | — | — | — | — | 1 (16.7) | — | — |
| Hypotension | 1 (11.1) | — | — | — | 1 (16.7) | — | — |
| Thromboembolic | 1 (11.1) | — | — | — | — | — | — |
| event | | | | | | | |

### Example 2

Phase 2 Clinical Trial—Trial to Assess the Safety and Efficacy of the Combination of SG and EV in Patients with Resistant mUC

### Study Design

This study is a multicenter phase 2 trial to test the efficacy of SG in combination with EV in treatment refractory urothelial carcinoma. Patients will receive a combination of EV 1.25 mg/kg and SG 7.5 mg/kg (D1, D8 every three weeks). Dose reductions of EV to 1, 0.75 and 0.5 mg/kg will be permitted while SG can be dose reduced 5 mg/kg. The efficacy evaluable population in this trial will consist of patients who complete at least one cycle of treatment and have tumor response evaluated after initiation of treatment.

### Primary Objective

To further refine the recommended dose of SG+EV combination and to assess the ORR (unconfirmed CR or PR) per RECIST Version 1.1 as determined by investigator for the combination of EV and SG in combination in patients with mUC progressing on platinum-based chemotherapy and PD1/L1 inhibitors.

### Secondary Objective

To evaluate progression-free survival per investigator assessment
To evaluate Overall Survival
To evaluate safety of combination therapy

### Correlative Objectives

To monitor changes in levels of ctDNA on therapy
Tumor immunohistochemistry for Trop-2, Nectin-4, PD-L1
Explore predictors of response through highly multiplexed immunohistochemistry and molecular studies

### Inclusion Criteria

Participants must have histologically documented confirmed predominant urothelial carcinoma (i.e. of the bladder, renal pelvis, ureter or urethra). Patients with squamous differentiation or mixed cell types are eligible if the urothelial component is more than 50%; small-cell carcinoma is not allowed. Patients with locally advanced unresectable disease are eligible.

Patient who are cisplatin eligible must have received prior treatment with platinum containing therapy defined as within the adjuvant/neoadjuvant setting with ≥ypT2 disease at surgery or recurrent or progressive disease within 12 months or receiving treatment with platinum in locally advanced or metastatic setting. In addition, they must have received a checkpoint inhibitor (CPI) in locally advanced or metastatic urothelial carcinoma setting. Patients who received CPI therapy in the neoadjuvant/adjuvant setting and had recurrent or progressive disease either during or within 12 months of therapy completion are eligible. A CPI is defined as a PD-1 or PD-L1 inhibitor, e.g., an anti-PD1 antibody or anti-PD-L1 antibody.

Patients who are cisplatin-ineligible need only have progressed on or since one line of therapy defined as therapy given in the adjuvant/neoadjuvant setting within 12 months of progression or receiving therapy for locally advanced or metastatic disease.

Patient must be progressing on or since most recent therapy.

Age ≥18 years.

ECOG performance status 0-1.

Participants must have adequate organ and marrow function as defined below:

| | |
|---|---|
| Leukocytes | ≥3,000/mcL |
| Absolute neutrophil count | ≥1,500/mcL |
| Platelets | ≥100,000/mcL |
| Total bilirubin | ≤ institutional upper |
| | limit of normal (ULN) |
| AST(SGOT)/ALT(SGPT) | ≤2.5x institutional ULN |
| | OR |
| | ≤5x ULN with liver |
| | metastases and serum albumin |
| | >3 g/dL |
| Glomerular filtration | ≥30 mL/min/1.73 m2 |
| rate (GFR) | (by Cockcroft Gault formula) |

Human immunodeficiency virus (HIV)-infected participants on effective antiretroviral therapy with undetectable viral load within 6 months are eligible for this trial.

For participants with evidence of chronic hepatitis B virus (HBV) infection, the HBV viral load must be undetectable on suppressive therapy, if indicated.

Participants with a history of hepatitis C virus (HCV) infection must have been treated and cured. For participants with HCV infection who are currently on treatment, they are eligible if they have an undetectable HCV viral load.

Participants with known history or current symptoms of cardiac disease, or history of treatment with cardiotoxic agents, should have a clinical risk assessment of cardiac function using the New York Heart Association Functional Classification. To be eligible for this trial, participants should be class 2B or better.

Have measurable disease by computed tomography (CT) or magnetic resonance imaging (MRI) as per response evaluation criteria in solid tumors version 1.1 (RECIST 1.1 criteria). Tumor lesions situated in a previously irradiated area are considered measurable if progression has been demonstrated in such lesions.

### Exclusion Criteria

Subjects meeting any of the following exclusion criteria at Screening/Day 1 of treatment will not be enrolled in the study.

Have had a prior anti-cancer biologic agent (including immune checkpoint inhibitors) within 4 weeks prior to Cycle 1 Day 1 (C1D1) or have had prior chemotherapy, targeted small molecule therapy, or radiation therapy within 2 weeks prior to C1D1. Subjects participating in observational studies are eligible.

Presence of any toxicities attributed to prior anti-cancer therapy that are not resolved to Grade 1 or baseline that could impose serious risk for complications before administration of study drug agent.

Have previously received topoisomerase 1 inhibitors, SG or EV

Have an active second malignancy. Subjects with a history of malignancy that have been completely treated, with no evidence of active cancer for 3 years prior to start of therapy on trial (Cycle 1 Day 1 [C1D1]), or subjects with surgically-cured tumors with low risk of recurrence are allowed to enroll.

Have known active central nervous system (CNS) metastases and/or carcinomatous meningitis. All subjects with carcinomatous meningitis are excluded regardless of clinical stability.

Have active cardiac disease, defined as:
Myocardial infarction or unstable angina pectoris within 6 months prior to C1D1
History of serious ventricular arrhythmia (i.e., ventricular tachycardia or ventricular fibrillation), high-grade atrioventricular block, or other cardiac arrhythmias requiring anti-arrhythmic medications (except for atrial fibrillation that is well controlled with antiarrhythmic medication); history of QT interval prolongation
New York Heart Association (NYHA) Class III or greater congestive heart failure or left ventricular ejection fraction of <40%

Have active chronic inflammatory bowel disease (ulcerative colitis, Crohn's disease) or gastrointestinal (GI) perforation within 6 months of C1D1

Have active serious infection requiring antibiotics (Contact sponsor PI or Co-PI for clarification)

Have other concurrent medical or psychiatric conditions that, in the Investigator's opinion, may be likely to confound study interpretation or prevent completion of study procedures and follow-up examinations.

Patients with conditions requiring high doses of steroids (>10 mg/day of prednisone or equivalent) or other immunosuppressive medications are excluded. Inhaled or topical steroids are permitted in the absence of active autoimmune disease.

History of idiopathic pulmonary fibrosis; organizing pneumonia, drug-induced pneumonitis, idiopathic pneumonitis, or evidence of active pneumonitis on screening chest CT scan.

Participants who are receiving any other investigational agents.

History of allergic reactions attributed to compounds of similar chemical or biologic composition to EV or SG or any excipient contained in the drug formulations (including 2-(N-morpholino) ethane sulfonic acid (MES), histidine, treahalose dihydrate polysorbate 80 and polysorbate 20).

Participants with uncontrolled intercurrent illness.

Patients with uncontrolled diabetes. Uncontrolled diabetes is defined as hemoglobin A1C>8% or 7-8% with associated diabetes symptoms that are otherwise not explained

Uncontrolled tumor related bone pain or impending spinal cord compression.

### Treatment Regimen in Phase II Study

Therapy will be administered in a 21-day cycle with SG and EV given IV on Days 1 and 8 of each 3-week cycle. Starting doses will be SG 7.5 mg/kg and EV 1.25 mg/kg IV respectively. EV is dosed at a maximum weight of 100 kg. Dose reductions of each agent can occur independently. SG can be reduced to 0.5 mg/kg and EV to 1, 0.75 mg/kg and 0.5 mg/kg. Both agents are administered IV on days 1 and 8 every 3 weeks (1 cycle) till progression or intolerable toxicities. Toxicities are captured up to 4 weeks after the last dose of therapy. Treatment will be administered on an outpatient basis. All drugs can be held independent of each other as needed.

| | SG | EV |
|---|---|---|
| Starting Dose | 7.5 mg/kg IV | 1.25 mg/kg (Max 125 mg) |
| | D1, 8 q 3 weeks | IV D1, 8 q 3 weeks |
| Dose reductions | | |
| −1 | 5 mg/kg IV D1, | 1 mg/kg (max 100 mg) |
| | 8 q 3 weeks | IV D1, 8 q 3 weeks |
| −2 | NA | 0.75 mg/kg (max 75 mg) |
| | | IV D1,8 q 3 weeks |
| −3 | NA | 0.5 mg/kg (max 50 mg) |
| | | IV D1,8 q 3weeks |

EV is administered before SG on days of infusion. Premedication for prevention of infusion-related reactions with antipyretics and H1 and H2 blockers should be administered before each sacituzumab govitecan infusion. This includes acetaminophen 650 mg PO, H1 Blocker such as Benadryl 25 mg PO/IV or Loratidine 10 mg PO; H2 blocker such as Pepcid 20 mg PO/IV). Premedications should be administered approximately 30 minutes before each sacituzumab govitecan infusion. Corticosteroids (hydrocortisone 50 mg or equivalent orally [PO] or IV) may be administered prior to subsequent infusions. Antiemetic premedication is required, using a 3-drug regimen including a 5-HT3 inhibitor (ondansetron or palonosetron, or other agents according to local practices), an NK1-receptor antagonist (fosaprepitant or aprepitant), and dexamethasone (12 mg PO or IV). Beginning with cycle 3 day 1, the dose of dexamethasone premedication may be reduced or eliminated per investigator discretion. Anticipatory nausea can be treated with olanzapine. Growth factors and other supportive care are allowed when medically necessary any time during treatment with SG, to include prophylactically before start of treatment in cycle 1 if meets ASCO guidelines. Patients who exhibit an excessive cholinergic response to SG treatment (e.g., abdominal cramping, diarrhea, salivation, etc.) can receive appropriate premedication (e.g., Atropine) for subsequent treatments. There are no premedications administered routinely before EV.

### Example 3

Phase 2 Clinical Trial—Trial to Assess the Safety and Efficacy of SG and EV in Combination with Pembrolizumab in Patients with Treatment Naive mUC Ineligible for or Refusing Cisplatin Based Therapy

### Study Design

The study is a phase 2 design to study EV+SG+Pembrolizumab in patients who are treatment naive or progressed over 12 months from completion of perioperative therapy and ineligible for cisplatin based therapy. They will receive a triplet regimen of EV 1.25 mg/kg, SG 7.5 mg/kg (D1 and D8 every three weeks), and pembrolizumab 200 mg (D1 every 3 weeks) or 400 mg (D1 every 6 weeks) as a front-line therapy. Dose reductions of EV to 1 and 0.75 mg/kg will be permitted while SG can be dose reduced to 5 mg/kg. No dose reductions of pembrolizumab are permitted.

Primary Objective: To assess the ORR (unconfirmed CR or PR) per RECIST Version 1.1 as determined by investigator for the combination therapy of pembrolizumab in combination with EV and SG in treatment-naive patients with mUC.

### Secondary Objective

To evaluate progression-free survival per investigator assessment
To evaluate overall Survival
To evaluate of the safety of triplet regimen

### Correlative Objectives

Tumor immunohistochemistry for Trop-2, Nectin-4, PD-L1
To monitor changes in levels of ctDNA on therapy
Explore predictors of response through highly multiplexed immunohistochemistry and molecular studies

### Inclusion Criteria

Participants must have histologically documented confirmed predominant urothelial carcinoma (i.e. of the bladder, renal pelvis, ureter or urethra). Patients with squamous differentiation or mixed cell types are eligible if the transitional component is more than 50%; small-cell carcinoma is not allowed. Patients with locally advanced unresectable disease are eligible.

No prior therapy for metastatic urothelial carcinoma. Therapy in the perioperative setting is allowed provided it has been >12 months since last dose of platinum base therapy and 6 months from last dose of immune checkpoint blockade if this was pursued.

Patients must be ineligible for or cisplatin based therapy with CrCl (calculated or measured) ≥30 and <60 mL/min, hearing loss/dysfunction, age, and/or allergy to cisplatin.

Age ≥18 years.

ECOG performance status 0-1.

Participants must have adequate organ and marrow function as defined below:

| | |
|---|---|
| Leukocytes | ≥3,000/mcL |
| Absolute neutrophil count | ≥1,500/mcL |
| Platelets | ≥100,000/mcL |
| Total bilirubin | ≤ institutional upper |
| | limit of normal (ULN) |
| AST(SGOT)/ALT(SGPT) | ≤2.5x institutional ULN |
| | OR |
| | ≤5x ULN with liver |
| | metastases and serum albumin |
| | >3 g/dL |
| Glomerular filtration | ≥30 mL/min/1.73 m2 |
| rate (GFR) | (by Cockcroft Gault formula) |

Human immunodeficiency virus (HIV)-infected participants on effective anti-retroviral therapy with undetectable viral load within 6 months are eligible for this trial.

For participants with evidence of chronic hepatitis B virus (HBV) infection, the HBV viral load must be undetectable on suppressive therapy, if indicated.

Participants with a history of hepatitis C virus (HCV) infection must have been treated and cured. For participants with HCV infection who are currently on treatment, they are eligible if they have an undetectable HCV viral load.

Participants with known history or current symptoms of cardiac disease, or history of treatment with cardiotoxic agents, should have a clinical risk assessment of cardiac function using the New York Heart Association Functional Classification. To be eligible for this trial, participants should be class 2B or better.

Have measurable disease by computed tomography (CT) or magnetic resonance imaging (MRI) as per response evaluation criteria in solid tumors version 1.1 (RECIST 1.1 criteria). Tumor lesions situated in a previously irradiated area are considered measurable if progression has been demonstrated in such lesions.

Female subjects of childbearing potential must have a negative urine or serum pregnancy test within 72 hours prior to receiving the first dose of study medication. If the urine test is positive or cannot be confirmed as negative, a serum pregnancy test will be required.

### Exclusion Criteria

Subjects meeting any of the following exclusion criteria at Screening/Day 1 of treatment will not be enrolled in the study.

Have had a prior anti-cancer biologic agent (including immune checkpoint inhibitors) or chemotherapy within 52 weeks prior to Cycle 1 Day 1 (C1D1) Subjects participating in observational studies are eligible.

Have previously received topoisomerase 1 inhibitors, SG or EV

Have an active second malignancy. Subjects with a history of malignancy that have been completely treated, with no evidence of active cancer for 3 years prior to start of therapy on trial (Cycle 1 Day 1 [C1D1]), or subjects with surgically-cured tumors with low risk of recurrence are allowed to enroll.

Have known active central nervous system (CNS) metastases and/or carcinomatous meningitis. Subjects with previously treated brain metastases may participate provided they have stable CNS disease for at least 4 weeks prior to the first dose of study drug and all neurologic symptoms have returned to baseline, have no evidence of new or enlarging brain metastases, and are taking ≤10 mg/day of prednisone or its equivalent. All subjects with carcinomatous meningitis are excluded regardless of clinical stability.

Have active cardiac disease, defined as:
Myocardial infarction or unstable angina pectoris within 6 months prior to C1D1
History of serious ventricular arrhythmia (i.e., ventricular tachycardia or ventricular fibrillation), high-grade atrioventricular block, or other cardiac arrhythmias requiring anti-arrhythmic medications (except for atrial fibrillation that is well controlled with antiarrhythmic medication); history of QT interval prolongation
New York Heart Association (NYHA) Class III or greater congestive heart failure or left ventricular ejection fraction of <40%

Have active chronic inflammatory bowel disease (ulcerative colitis, Crohn's disease) or gastrointestinal (GI) perforation within 6 months of C1D1

Have active serious infection requiring antibiotics (Contact sponsor PI or Co-PI for clarification)

Have other concurrent medical or psychiatric conditions that, in the Investigator's opinion, may be likely to confound study interpretation or prevent completion of study procedures and follow-up examinations.

Patients with conditions requiring high doses of steroids (>10 mg/day of prednisone or equivalent) or other immunosuppressive medications are excluded. Inhaled or topical steroids are permitted in the absence of active autoimmune disease.

History idiopathic pulmonary fibrosis; organizing pneumonia, drug-induced pneumonitis, idiopathic pneumonitis, or evidence of active pneumonitis on screening chest CT scan.

3.6.12 Participants who are receiving any other investigational agents.

3.6.13 History of allergic reactions attributed to compounds of similar chemical or biologic composition to pembrolizumab, EV or SG or any excipient contained in the drug formulations (including 2-(N-morpholino) ethane sulfonic acid (MES), histidine, treahalose dihydrate polysorbate 80 and polysorbate 20).

3.6.14 Participants with uncontrolled intercurrent illness.

3.6.15 Participants with psychiatric illness/social situations that would limit compliance with study requirements.

3.6.16 Patients with uncontrolled diabetes. Uncontrolled diabetes is defined as hemoglobin A1C>8% or 7-8% with associated diabetes symptoms that are otherwise not explained.

3.6.17 Uncontrolled tumor related bone pain or impending spinal cord compression.

3.6.18 History of autoimmune disease that has required systemic treatment in the past 2 years (i.e., with use of disease modifying agents, corticosteroids, or immunosuppressive drugs).
1. Replacement therapy (e.g., thyroxine, insulin, physiologic corticosteroid replacement therapy for adrenal or pituitary insufficiency) is not considered a form of systemic treatment and is allowed.
2. Brief (<7 days) use of systemic corticosteroids is allowed when use is considered standard of care.
3. Subjects with vitiligo, psoriasis, type 1 diabetes mellitus, hypothyroidism, or resolved childhood asthma/atopy will not be excluded.
4. Subjects requiring intermittent use of bronchodilators, inhaled steroids, or local steroid injections will not be excluded.
5. Subjects with hypothyroidism that is stable with hormone replacement or Sjogren's syndrome will not be excluded.

3.7 Treatment Regimen Therapy will be administered in a 21-day cycle with SG and EV given IV on Days 1 and 8 of each 3-week cycle. Starting doses will be SG 7.5 mg/kg and EV 1.25 mg/kg IV respectively. EV is dosed at a maximum weight of 100 kg. SG can be reduced to 5 mg/kg and EV to 1 and 0.75 and 0.5 mg/kg mg/kg. SG and EV administered IV on days 1 and 8 every 3 weeks (1 cycle) till progression or intolerable toxicities. Pembrolizumab will be dosed at 200 mg IV D1 through C6 and then 400 mg on D1 every 6 weeks from C7 (on odd cycles) onwards; dose delays to D8 are permitted as needed for toxicity with a minimum io 3 weeks (−2 days) between doses for 200 mg dose and 6 weeks (−2 days) between doses for 400 mg dose. Toxicities are captured up to 4 weeks after the last dose of therapy.

| | SG | EV | Pembrolizumab |
|---|---|---|---|
| Starting | 7.5 mg/kg IV | 1.25 mg/kg (Max 125 | 200 mg IV every 3 weeks for |
| Dose | D1, 8 q 3 weeks | mg) IV D1, 8 q 3 weeks | 6 cycles then 400 mg IV every |
| | | | 6 weeks |
| Dose reductions | | | |
| −1 | 5 mg/kg IV | 1 mg/kg (max 100 mg) | NA |
| | D1, 8 q 3 weeks | IV D1, 8 q 3 weeks | |
| −2 | NA | 0.75 mg/kg (max 75 mg) | NA |
| | | IV D1, 8 q 3 weeks | |
| −3 | NA | 0.5 mg/kg (max 50 mg) | NA |
| | | IV D1, 8 q 3 weeks | |

EV is administered first. Pembrolizumab 200 mg will be administered as an IV infusion approximately 60 minutes after completion of enfortumab vedotin during cylces 1-3 and approximately 30 minutes after completion of enfortumab vedotin after cycle 3. A delay of 15 minutes may be used for subsequent infusions if the previous infusion was well tolerated. SG can then be administered. Premedication for prevention of infusion-related reactions with antipyretics and H1 and H2 blockers should be administered before each sacituzumab govitecan infusion. This includes acetaminophen 650 mg PO, H1 Blocker such as Benadryl 25 mg PO/IV or Loratidine 10 mg PO; H2 blocker such as Pepcid 20 mg PO/IV). Premedications should be administered approximately 30 minutes before each sacituzumab govitecan infusion. Corticosteroids (hydrocortisone 50 mg or equivalent orally [PO] or IV) may be administered prior to subsequent infusions. Antiemetic premedication is required, using a 3-drug regimen including a 5-HT3 inhibitor (ondansetron or palonosetron, or other agents according to local practices), an NK1-receptor antagonist (fosaprepitant or aprepitant), and dexamethasone (12 mg PO or IV). Beginning with cycle 3 day 1, the dose of dexamethasone premedication may be reduced or eliminated per investigator discretion. Anticipatory nausea can be treated with olanzapine. Growth factors and other supportive care are allowed when medically necessary any time during treatment with SG, to include prophylactically before start of treatment in cycle 1 if meets ASCO guidelines. Patients who exhibit an excessive cholinergic response to SG treatment (e.g., abdominal cramping, diarrhea, salivation, etc.) can receive appropriate premedication (e.g., Atropine) for subsequent treatments. There are no premedications administered routinely before EV.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1-6. (canceled)

7. A method of treating relapsed or refractory locally advanced or metastatic urothelial carcinoma in a human subject, comprising administering to the subject a combination therapy which comprises:
(i) a therapeutically effective amount of a Trop-2 ADC and
(ii) a therapeutically effective amount of a Nectin-4 ADC;
wherein the combination therapy does not comprise an anti-PD1 antibody or an anti-PD-L1 antibody, and
wherein the subject has received one or more prior therapies for locally advanced or metastatic urothelial carcinoma.

8. A method of treating locally advanced or metastatic urothelial carcinoma in a human subject, comprising administering to the subject a combination therapy which comprises:
(i) a therapeutically effective amount of a Trop-2 ADC;
(ii) a therapeutically effective amount of a Nectin-4 ADC, and
(iii) a therapeutically effective amount of an anti-PD1 antibody or an anti-PD-L1 antibody;
wherein the subject has not received a prior therapy for locally advanced or metastatic urothelial carcinoma.

9-29. (canceled)

30. The method of claim 7, wherein the Trop-2 ADC is SG.

31-37. (canceled)

38. The method of claim 1, wherein the Nectin-4 ADC is enfortumab vedotin (EV).

39. (canceled)

40. The method of claim 1, wherein the Trop-2 ADC is administered at a dose of from 5 mg/kg to 10 mg/kg as an intravenous infusion on day 1 and day 8 of a 21-day treatment cycle.

41-45. (canceled)

46. The method of claim 1, wherein the Nectin-4 ADC is administered at a dose of from 0.5 mg/kg to 1.25 mg/kg as an intravenous infusion on day 1 and day 8 a 21-day treatment cycle.

47-56. (canceled)

57. The method of claim 1, wherein
the Trop-2 ADC is SG; and SG is administered at a dose of 8 mg/kg as an intravenous infusion on day 1 and day 8 of a 21-day treatment cycle; and
the Nectin-4 ADC is EV; and EV is administered as an intravenous infusion at a dose of 1.25 mg/kg on day 1 and day 8 of the 21-day treatment cycles.

58-65. (canceled)

66. The method of claim 7, wherein the urothelial carcinoma is relapsed or refractory locally advanced or metastatic urothelial carcinoma.

67. The method of claim 7, wherein the subject is treatment naïve.

68-69. (canceled)

70. The method of claim 7, wherein the subject has received one or more prior therapies for metastatic or locally advanced urothelial carcinoma.

71. (canceled)

72. The method of claim 7, wherein the subject has received prior platinum-based therapy for metastatic urothelial carcinoma, and the metastatic urothelial carcinoma has progressed during or after the platinum-based therapy.

73. The method of claim 7, wherein the subject has received prior immunotherapy for metastatic urothelial carcinoma, and the metastatic urothelial carcinoma has progressed during or after the immunotherapy.

74-92. (canceled)

93. The method of claim 7, wherein the combination therapy is administered for 10 or more, 11 or more, 12 or more, 13 or more, 14 or more, 15 or more, 16 or more, 17 or more, 18 or more, 19 or more, or 20 or more treatment cycles.

94. The method of claim 7, wherein the combination therapy is administered for 14 or more treatment cycles.

95. The method of claim 7, wherein when the method is used to treat a population of subjects having locally advanced or metastatic UC, the method achieves an overall response rate (ORR) of at least 60%, at least 65%, or at least 70%.

96. The method of claim 7, wherein when the method is used to treat a population of subjects having locally advanced or metastatic UC, the method achieves an overall response rate (ORR) of 60-85%, 65-85%, 70-85%, 70-80%, or 70-75%.

97. The method of claim 7, wherein the Trop-2 ADC is SG and the Nectin-4 ADC is EV, wherein when the method is used to treat a population of subjects, the method achieves an overall response rate (ORR) of at least 60% at a median follow-up of 14 months or more.

98. The method of claim 7, wherein when the method is used to treat a population of subjects, the method achieves a higher ORR compared to administering the Trop-2 ADC or the Nectin-4 ADC as a reference monotherapy to a population of reference subjects.

99. (canceled)

100. The method of claim 7, wherein when the method is used to treat a population of subjects, the method achieves a median duration of response (DOR) of greater than 12 months.

101-143. (canceled)

144. The method of claim 7, wherein the subject has received prophylactic granulocyte colony stimulating factor (G-CSF).

145-153. (canceled)
